SEQUENCE LISTING (84 sequences)

SEQ ID NO: 1 - DNA, 366 bp, Artificial Sequence
  Description of Artificial Sequence Synthetic polynucleotide
caggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggcaagtcactgtcagc

SEQ ID NO: 2 - DNA, 369 bp, Artificial Sequence
  Description of Artificial Sequence Synthetic polynucleotide
caggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagc

SEQ ID NO: 3 - DNA, 15 bp, Artificial Sequence
  Description of Artificial Sequence Synthetic oligonucleotide
tctggtggtggttcc

SEQ ID NO: 4 - DNA, 21 bp, Artificial Sequence
  Description of Artificial Sequence Synthetic oligonucleotide
ggaggatctggtggtggttcc

SEQ ID NO: 5 - DNA, 51 bp, Artificial Sequence
  Description of Artificial Sequence Synthetic oligonucleotide
ggaggatccggtggtggaggatcatctggtggaggatctggtggtggttcc

SEQ ID NO: 6 - DNA, 105 bp, Artificial Sequence
  Description of Artificial Sequence Synthetic polynucleotide
ggtggaggtggttccggaggaggaagttccggtggtggatcttcttctggaggtggaggatccggtggtggaggatcatctggtggaggatctggtggtggttcc

SEQ ID NO: 7 - DNA, 498 bp, Artificial Sequence
  Description of Artificial Sequence Synthetic polynucleotide
gctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 8 - DNA, 498 bp, Artificial Sequence
  Description of Artificial Sequence Synthetic polynucleotide
gctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgg

SEQ ID NO: 9 - DNA, 70 bp, Artificial Sequence
  Description of Artificial Sequence Synthetic oligonucleotide
cgggcccgaacaaaaactcatctcagaagaggatctgaatagcgccgtcgaccatcatcatcatcatcat

SEQ ID NO: 10 - protein, 122 aa, Artificial Sequence
  Description of Artificial Sequence Synthetic polypeptide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGQVTVS

SEQ ID NO: 11 - protein, 123 aa, Artificial Sequence
  Description of Artificial Sequence Synthetic polypeptide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVS

SEQ ID NO: 12 - protein, 5 aa, Artificial Sequence
  Description of Artificial Sequence Synthetic peptide
SGGGS

SEQ ID NO: 13 - protein, 7 aa, Artificial Sequence
  Description of Artificial Sequence Synthetic peptide
GGSGGGS

SEQ ID NO: 14 - protein, 17 aa, Artificial Sequence
  Description of Artificial Sequence Synthetic peptide
GGSGGGGSSGGGSGGGS

SEQ ID NO: 15 - protein, 35 aa, Artificial Sequence
  Description of Artificial Sequence Synthetic polypeptide
GGGGSGGGSSGGGSSSGGGGSGGGGSSGGGSGGGS

SEQ ID NO: 16 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 17 - protein, 166 aa, Artificial Sequence
  Description of Artificial Sequence Synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLAGKLKLYTGEACRTGDR

SEQ ID NO: 18 - protein, 23 aa, Artificial Sequence
  Description of Artificial Sequence Synthetic peptide
GPEQKLISEEDLNSAVDHHHHHH

SEQ ID NO: 19 - DNA, 51 bp, Artificial Sequence
  Description of Artificial Sequence Synthetic oligonucleotide
ggtggaggtggatctggtggtggaggatcttcaggaggtggtggatcttcc

SEQ ID NO: 20 - protein, 17 aa, Artificial Sequence
  Description of Artificial Sequence Synthetic peptide
GGGGSGGGGSSGGGGSS

SEQ ID NO: 21 - DNA, 87 bp, Artificial Sequence
  Description of Artificial Sequence Synthetic oligonucleotide
ggaggaagttccggtggtggatcttcttctggaggtggaggatccggtggtggaggatcatctggtggaggatctggtggtggttcc

SEQ ID NO: 22 - protein, 29 aa, Artificial Sequence
  Description of Artificial Sequence Synthetic peptide
GGSSGGGSSSGGGGSGGGGSSGGGSGGGS

SEQ ID NO: 23 - DNA, 117 bp, Artificial Sequence
  Description of Artificial Sequence Synthetic polynucleotide
ggtggaagtagtggtggaggtggttccggaggaggaagttccggtggtggatcttcttctggaggtggaggatccggtggtggaggatcatctggtggaggatctggtggtggttcc

SEQ ID NO: 24 - protein, 39 aa, Artificial Sequence
  Description of Artificial Sequence Synthetic polypeptide
GGSSGGGGSGGGSSGGGSSSGGGGSGGGGSSGGGSGGGS

SEQ ID NO: 25 - protein, 4 aa, Homo sapiens
YPPE

SEQ ID NO: 26 - protein, 150 aa, Homo sapiens
MYGKIIFVLLLSEIVSISALSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 27 - protein, 11 aa, Artificial Sequence
  Description of Artificial Sequence Synthetic peptide
SGYTDSTYCVG

SEQ ID NO: 28 - protein, 17 aa, Artificial Sequence
  Description of Artificial Sequence Synthetic peptide
RINTISGRPWYADSVKG

SEQ ID NO: 29 - protein, 15 aa, Artificial Sequence
  Description of Artificial Sequence Synthetic peptide
TTANSRGFCSGGYNY

SEQ ID NO: 30 - DNA, 1943 bp, Artificial Sequence
  Description of Artificial Sequence Synthetic polynucleotide
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgggggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 31 - DNA, 5930 bp, Artificial Sequence
  Description of Artificial Sequence Synthetic polynucleotide
acggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgggggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcg

SEQ ID NO: 32 - DNA, 5497 bp, Artificial Sequence
  Description of Artificial Sequence Synthetic polynucleotide
aaagtgccacctgacgtcgacggatcgggagatcagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccaaccctttaatacgactcactatagggagacccaagctggctagccaccatggagacagacacactcctgctatgggtactgctgctctgggttccaggttccactggtcaggtccaactgcaagagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgggggcccgaacaaaaactcatctcagaagaggatctgaatagcgccctggttccgagaggctcccgtctcgtcgaccatcatcatcatcatcattgagtttaaacccgctgaaagggttggatccctaccggtgctgcggccgcgcagttaacgccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaagatctgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaaaaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccattaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccga

SEQ ID NO: 33 - protein, 11 aa, Homo sapiens
AATPRAHEVSE

SEQ ID NO: 34 - protein, 10 aa, Homo sapiens
ATPRAHEVSE

SEQ ID NO: 35 - protein, 5 aa, Homo sapiens
PRAHE

SEQ ID NO: 36 - protein, 6 aa, Homo sapiens
PRAHEV

SEQ ID NO: 37 - protein, 7 aa, Homo sapiens
PRAHEVS

SEQ ID NO: 38 - protein, 8 aa, Homo sapiens
PRAHEVSE

SEQ ID NO: 39 - protein, 10 aa, Homo sapiens
ISVRTVYPPE

SEQ ID NO: 40 - protein, 8 aa, Homo sapiens
RTVYPPEE

SEQ ID NO: 41 - protein, 5 aa, Homo sapiens
YPPEE

SEQ ID NO: 42 - protein, 6 aa, Homo sapiens
YPPEEE

SEQ ID NO: 43 - protein, 7 aa, Homo sapiens
YPPEEET

SEQ ID NO: 44 - protein, 5 aa, Homo sapiens
PPEEE

SEQ ID NO: 45 - protein, 6 aa, Homo sapiens
PPEEET

SEQ ID NO: 46 - protein, 242 aa, Artificial Sequence
  Description of Artificial Sequence Synthetic polypeptide
QVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVSSGGGGSGGGGSSGGGGSSDIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIK

SEQ ID NO: 47 - protein, 118 aa, Artificial Sequence
  Description of Artificial Sequence Synthetic polypeptide
EVRLLESGGGPVQPGGSLKLSCAASGFDFSRYWMNWVRRAPGKGLEWIGEINQQSSTINYSPPLKDKFIISRDNAKSTLYLQMNKVRSEDTALYYCARLSLTAAGFAYWGQGTLVTVS

SEQ ID NO: 48 - protein, 112 aa, Artificial Sequence
  Description of Artificial Sequence Synthetic polypeptide
DIVMSQSPSSLAVSVGEKVSMSCKSSQSLFNSRTRKNYLTWYQQKPGQSPKPLIYWASTRESGVPDRFTGSGSGTDFTLTISSVQAEDLADYYCKQSYNLRTFGGGTKLEIK

SEQ ID NO: 49 - protein, 118 aa, Artificial Sequence
  Description of Artificial Sequence Synthetic polypeptide
QVKLQQSGGGLVQPGGSLKLSCAASGFTFSSYGMSWFRQTPDKRLELVAIINSNGGTTYYPDSVKGRFTISRDNAKNTLYLQMSSLKSEDTAMYYCARGGGRWLLDYWGQGTTVTVSS

SEQ ID NO: 50 - protein, 112 aa, Artificial Sequence
  Description of Artificial Sequence Synthetic polypeptide
DIELTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSSTFGGGTKLEIK

SEQ ID NO: 51 - protein, 5 aa, Artificial Sequence
  Description of Artificial Sequence Synthetic peptide
GGGGS

SEQ ID NO: 52 - protein, 6 aa, Artificial Sequence
  Description of Artificial Sequence Synthetic 6xHis tag
HHHHHH

SEQ ID NO: 53 - protein, 85 aa, Homo sapiens
LSTTEVAMHTSTSSSVTKSYISSQTNDTHKRDTYAATPRAHEVSEISVRTVYPPEEETGERVQLAHHFSEPEITLIIFGVMAGVI

SEQ ID NO: 54 - protein, 5 aa, Artificial Sequence
  Description of Artificial Sequence Synthetic peptide
KLEIK

SEQ ID NO: 55 - protein, 5 aa, Artificial Sequence
  Description of Artificial Sequence Synthetic peptide
QVTVS

SEQ ID NO: 56 - protein, 5 aa, Artificial Sequence
  Description of Artificial Sequence Synthetic peptide
VTVSS

SEQ ID NO: 57 - DNA, 10 bp, Unknown
  Description of Unknown Kozak sequence
gccrccatgg

SEQ ID NO: 58 - protein, 5 aa, Homo sapiens
YAATP

SEQ ID NO: 59 - protein, 4 aa, Homo sapiens
RTVY

SEQ ID NO: 60 - DNA, 366 bp, Artificial Sequence
  Description of Artificial Sequence Synthetic polynucleotide
caggtccaactgcaagagagcggaggagggtctgttcaagctggcggttccctccggctttcttgcgtggcgtcaggctatactgacagcacatactgcgtgggctggttcaggcaggcccctggaaaggagcgcgagggcgtagcccgcataaatactatatctggcagaccgtggtacgctgacagcgtgaagggacggtttacaatcagtcaagataactctaaaaacaccgtgtttcttcaaatgaattctttgaaacccgaagatactgccatctattattgcacacttacgaccgcgaactcacgcggtttttgtagcggaggatataactataaagggcaagggcaggtaactgtatcc

SEQ ID NO: 61 - DNA, 726 bp, Artificial Sequence
  Description of Artificial Sequence Synthetic polynucleotide
caagttaagttgcaacaatctggtgctgaattggttaagccaggtgcttctgttaagttgtcttgtaaggcttctggttacaccttcaactcttactttatgcattggatgaagcaaagaccagttcaaggtttggaatggattggtatgattagaccaaacggtggtactaccgattacaacgagaagtttaagaacaaggctactttgactgttgataagtcctctaacactgcttacatgcaattgaactctttgacttctggtgattctgctgtttactactgtgctagatgggaaggttcttactacgctttggattactggggtcaaggtaccactgttactgtttcttccggtggaggtggatctggtggtggaggatcttcaggaggtggtggatcttccgatattgagttgactcaatctccagctattatgtctgctaccttgggtgagaaggttactatgacttgtagagcttcatctaacgttaagtacatgtactggtaccaacagaagtctggtgcttctccaaagttgtggatttactacacttctaacttggcttctggtgttccaggtagattttctggttcaggttctggtacttcctactctttgactatttcctctgttgaagctgaagatgctgctacttactactgtcaacaattcacttcttccccatacacttttggaggaggtactaagttggaaatcaag

SEQ ID NO: 62 - DNA, 747 bp, Artificial Sequence
  Description of Artificial Sequence Synthetic polynucleotide
gaagtccgtctgctggaaagcgggggtggtcctgtgcagcctggtgggtccctgaaactgtcctgtgccgcaagcgggttcgatttttccagatactggatgaactgggtgaggagggctccaggcaagggcctggagtggatcggcgagatcaaccagcagtccagcaccatcaattactctccccctctgaaggacaagttcatcatcagccgcgataacgctaagtctacactgtatctgcagatgaataaggtgagaagcgaggacaccgccctgtactattgcgctcgcctgtctctgacagccgctggctttgcctattggggccagggcaccctggtgacagtgtctgctggaggaggctcttccggaggatccggcagctctggcggctccagctctggcggcgatatcgtgatgagccagtctccctccagcctggccgtgtccgtgggagagaaggtgtccatgagctgtaagtcttcccagtctctgttcaactccagaacccgcaagaattacctgacatggtatcagcagaagcctggccagagccccaagcctctgatctactgggccagcaccagagagtctggagtgccagaccgcttcaccggctctggatccggcacagacttcaccctgacaatcagctctgtgcaggccgaggacctggctgattactattgcaagcagtcctataatctgaggacctttggcggcggcacaaagctggagatcaag

SEQ ID NO: 63 - DNA, 741 bp, Artificial Sequence
  Description of Artificial Sequence Synthetic polynucleotide
caggttaaactccagcaaagtggtggcgggctcgtacaaccaggcggttccctcaagttgtcctgcgccgcatcagggtttacatttagctcttatggtatgtcttggtttcgccagacgcctgacaagcgactcgagctggtcgctatcatcaatagtaacggaggtactacatattatcccgacagtgtgaaggggcgatttaccattagccgggacaacgccaaaaatacactgtacctccagatgtcaagcttgaaatcagaagatacggccatgtactattgcgctagggggggtggaaggtggcttctggactattatggtcagggtacaacagtgacagtatcctccggtggaggtggatctggtggtggaggatcttcaggaggtggtggatcttccgacatagagcttacacaatctccgtcatcactggcagtctcagccggggaaaaagtgacaatgtcatgcaagtcaagccagagcgttctttattcatctaatcagaagaactacctggcatggtatcagcagaagccgggacagtcccctaagctcctcatctactgggcaagcaccagggaatccggagtgccggacaggtttactgggtccggttctgggacggattttacgcttacgatatcaagtgtccaagctgaggacctcgcagtatactactgtcaccagtacctgtcttcttctacttttgggggtggaacgaaactggaaataaaa

SEQ ID NO: 64 - DNA, 498 bp, Artificial Sequence
  Description of Artificial Sequence Synthetic polynucleotide
gctccacctagattgatttgtgattccagagttttggaaagatacttgttggaagctaaggaggctgaaaatattactactggttgtgctgaacattgttctttgaacgagaatattactgttccagatactgatgttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 65 - protein, 122 aa, Artificial Sequence
  Description of Artificial Sequence Synthetic polypeptide
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGQVTVS

SEQ ID NO: 66 - protein, 249 aa, Artificial Sequence
  Description of Artificial Sequence Synthetic polypeptide
EVRLLESGGGPVQPGGSLKLSCAASGFDFSRYWMNWVRRAPGKGLEWIGEINQQSSTINYSPPLKDKFIISRDNAKSTLYLQMNKVRSEDTALYYCARLSLTAAGFAYWGQGTLVTVSAGGGSSGGSGSSGGSSSGGDIVMSQSPSSLAVSVGEKVSMSCKSSQSLFNSRTRKNYLTWYQQKPGQSPKPLIYWASTRESGVPDRFTGSGSGTDFTLTISSVQAEDLADYYCKQSYNLRTFGGGTKLEIK

SEQ ID NO: 67 - protein, 247 aa, Artificial Sequence
  Description of Artificial Sequence Synthetic polypeptide
QVKLQQSGGGLVQPGGSLKLSCAASGFTFSSYGMSWFRQTPDKRLELVAIINSNGGTTYYPDSVKGRFTISRDNAKNTLYLQMSSLKSEDTAMYYCARGGGRWLLDYYGQGTTVTVSSGGGGSGGGGSSGGGGSSDIELTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSSTFGGGTKLEIK

SEQ ID NO: 68 - protein, 166 aa, Artificial Sequence
  Description of Artificial Sequence Synthetic polypeptide
APPRLICDSRVLERYLLEAKEAENITTGCAEHCSLNENITVPDTDVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 69 - protein, 131 aa, Homo sapiens
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 70 - protein, 131 aa, Pan troglodytes
ASSTTEVAMHTSTSSVTKSYISSETSDKHKWDIYPATPRAHEVSEIYVTTVYPPEEENGEGVQLVHRFSEPEITLIIFGVMAGVIGTILLIYYSICRLIKKSPSDVKPLPSPDTDVPLSSVEIENPETSDQ

SEQ ID NO: 71 - protein, 131 aa, Pongo sp.
SNTTGEVMHTSISSSVTKSYITPQTNDKHKQDTYPATPSAHEVSEISVITIHSPEEENGERGQLVHRFSEPVITLIVFGVMAGVIGTILLISYCIRRLRKQSPSDVQPLPSPDTDVPLSSVEIENPETIDQ

SEQ ID NO: 72 - protein, 131 aa, Gorilla sp.
WSTTEVAMHTSTSSSVTKSYISSQTNAKHKRDTYAAPPRAHEVSEISVTAVYPPEEYNGERGQLVHRFPEAEITLIIFGVMAGIIGTILLISYSIRRLIKKSPSDVKPRPSTDTDVPLSSVEIENPETSDQ

SEQ ID NO: 73 - protein, 130 aa, Pan paniscus
SSTTEVAMHTSTSSVTKSYISSETSDKQKWDTYPATPRAHEVSEIYVTTVYPPEEENGEGVQLVHRFSEPEITLIIFGVMAGVIGTILLIYYSICRLIKKSPSDVKPLPSPDTDVPLSSVEIENPETSDQ

SEQ ID NO: 74 - protein, 133 aa, Macaca mulatta
ASSTTVPATHTSTSSLGPESYVSSQSNDKHTSDTHPTTPSAHEVSEFSGRTHYPPEEDNRERVQLVHEFSELVIALIIFGVMAGVIGTILFISYCIRRLRKKSQSDVQPLPPPDAEVPLSSVEIENPEETDQS

SEQ ID NO: 75 - protein, 144 aa, Macaca fascicularis
ASSTTVPATHTSTSSLDPESYVSSQSNDKHTSDTHPTTPSAHEVSEFSGRTHYPPEEDNRERVQLVHEFSELVIALIIFGVMAGVIGTILFISYCIRRLRKKSQSDVQPLPPPDAEVPLSSVEIENPEETDQSNLFTKPNEERT

SEQ ID NO: 76 - protein, 131 aa, Rhinopithecus sp.
ASSTTVPEMHTSTSSLVPGSYVSSRPNDKHKWDIHPTSPSAHEVSEFSGRNHYPTEEENREREQLVHEFSELVIALIIFGVMAGVIGTILFISYCIRRLRKKSPSDVQPLPPPDAEVPLSSVEIENPGAGV

SEQ ID NO: 77 - protein, 123 aa, Artificial Sequence; identical to SEQ ID NO: 11
  Description of Artificial Sequence Synthetic polypeptide

SEQ ID NO: 78 - protein, 122 aa, Artificial Sequence; identical to SEQ ID NO: 65
  Description of Artificial Sequence Synthetic polypeptide

SEQ ID NO: 79 - protein, 126 aa, Artificial Sequence
  Description of Artificial Sequence Synthetic polypeptide
QVQLVESGGGSVQAGGSLRLSCAASGYTVSTYCMGWFRQAPGKEREGVATILGGSTYYGDSVKGRFTISQDNAKNTVYLQMNSLKPEDTAIYYCAGSTVASTGWCSRLRPYDYHYRGQGTQVTVSS

SEQ ID NO: 80 - protein, 125 aa, Artificial Sequence
  Description of Artificial Sequence Synthetic polypeptide
QVQLQESGGGSVQAGGSLRLSCTASGYTYRKYCMGWFRQAPGKEREGVACINSGGGTSYYADSVKGRFTISQDNAKDTVFLRMNSLKPEDTAIYYCALSSNSVCPPGHVAWYNDWGQGTQVTVSS

SEQ ID NO: 81 - protein, 128 aa, Artificial Sequence
  Description of Artificial Sequence Synthetic polypeptide
QVQLQESGGGLVQAGGSLRLSCAASGFTLDSYAIGWFRQAPGKEREGVSCISASGGSTNYADSVKGRFTISRDNAKNTVYLQMNSLKSEDTAVYYCAADHPGLCTSESGRRRYLEVWGQGTQVTVSSA

SEQ ID NO: 82 - protein, 115 aa, Artificial Sequence
  Description of Artificial Sequence Synthetic polypeptide
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 83 - protein, 126 aa, Artificial Sequence
  Description of Artificial Sequence Synthetic polypeptide
GQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSH

SEQ ID NO: 84 - protein, 121 aa, Artificial Sequence
  Description of Artificial Sequence Synthetic polypeptide
QVQLQESGGGSVQAGGSLRLSCSASGYTYISGWFRQAPGKEREGVAAIRSSDGTTYYADSVKGRFTISQDNAKNTVYLQMNSLKPEDTAMYYCAATEVAGWPLDIGIYDYWGQGTEVTVSS

(19) United States
(12) Patent Application Publication
(10) Pub. No.: US 2022/0098261 A1
(43) Pub. Date: Mar. 31, 2022

(54) METHODS AND COMPOSITIONS RELATING TO IMPROVED FORMS OF TARGETED ERYTHROPOIETIN

(71) Applicant: PRESIDENT AND FELLOWS OF HARVARD COLLEGE, Cambridge, MA (US)

(72) Inventors: Jungmin LEE, BOSTON, MA (US); Jeffrey Charles WAY, Cambridge, MA (US)

(73) Assignee: PRESIDENT AND FELLOWS OF HARVARD COLLEGE, Cambridge, MA (US)

(21) Appl. No.: 17/312,523

(22) PCT Filed: Dec. 19, 2019
(86) PCT No.: PCT/US2019/067459

§ 371 Date: Jun. 10, 2021

Related U.S. Application Data
(60) Provisional application No. 62/874,547, filed on Jul. 16, 2019.
(60) Provisional application No. 62/781,948, filed on Dec. 19, 2018.

(51) Int. Cl. C07K 14/505; C07K 16/28; A61P 7/06

(52) U.S. Cl. CPC C07K 14/505; C07K 16/2896; A61K 38/00; C07K 2319/33; A61P 7/06

(57) ABSTRACT

The technology described herein is directed to engineered polypeptides comprising an anti-GYPA antibody reagent and an EPO polypeptide. Further provided herein are methods of treating anemia by administering said polypeptides.

### CROSS-REFERENCE TO RELATED APPLICATIONS

This application claims benefit under 35 U.S.C. § 119(e) of U.S. Provisional Application Nos. 62/781,948 filed Dec. 19, 2018 and 62/874,547 filed Jul. 16, 2019, the contents of which are incorporated herein by reference in their entirety.

### GOVERNMENT SUPPORT

This invention was made with government support under Grant Nos. R01 GM036373 and 5U54HL119145-04, subaward 112475 awarded by the National Institutes of Health and Grant No. W911NF-11-2-0056 awarded by the Defense Advanced Research Projects Agency. The government has certain rights in the invention.

### TECHNICAL FIELD

The technology described herein relates to engineered forms of erythropoietin, e.g., targeted erythropoietin and methods of using such compositions.

### BACKGROUND

Erythropoietin (“EPO”) is used to treat anemia that can result from, e.g., kidney failure, cancer chemotherapy, inflammation of chronic disease, and so on. Pharmaceutical companies have developed a variety of forms of erythropoietin, including Epogen™ (epoetin alpha) and Procrit™ (also epoetin alpha), Aranesp™ (darbepoetin) and Hematide (a dimeric peptide with EPO activity, conjugated to a polyethylene-type polymer; this has been withdrawn from the market). These molecules vary in their pharmacokinetic properties and dosing regimens. The epoetin and darbepoetin molecules are qualitatively similar in that their receptor binding surfaces are identical. Epoetin and darbepoetin differ only in their pattern of glycosylation: darbepoetin has two additional N-linked glycosylation sites, which have the effect of reducing loss through the kidney, and also reducing receptor binding. The latter effect reduces receptor-mediated endocytosis and thus slows disappearance of darbepoetin in vivo. As a result, darbepoetin has a longer plasma half-life than epoetin and requires less frequent dosing.

The effect of these drugs may be measured by the hematocrit (the percent of blood volume that consists of red blood cells) or by reticulocytes. The latter are newly created red blood cells that still retain RNA after their final differentiation

Unfortunately, erythropoietin and its derivatives appear to have pro-thrombotic and pro-oncogenic effects. For example, Pfeffer et al (NEJM 361:2019-2032 (2009)), performed a large clinical trial (“TREAT”) that compared survival of diabetic kidney patients treated with darbepoetin or placebo. The treated group suffered from fatal or nonfatal stroke about twice as often as the placebo group. Besareb et al. (NEJM 339:584-590 (1998)) compared kidney dialysis patients treated with epoetin with a target hematocrit of either 30 or 42. They found that the group with the hematocrit target of 42 has a 30% higher death rate and a higher rate of first non-fatal myocardial infarctions.

Previous workers have developed approaches for targeting the activity of signaling molecules such as hormones and cytokines to specific tissues. In one approach, a signaling protein is fused to a targeting element, such as an antibody, that binds to a cell surface receptor expressed in a cell type or tissue type of interest. Prior binding of the antibody element to its cell surface receptor concentrates the signaling protein domain on a target cell surface, such that this signaling element activates its cognate receptor preferentially on these cells, as compared to cells without the antigen bound by the targeting element/antibody. Typically, such a fusion protein consists of a targeting element, an activity element, and a linker between the targeting element and activity element that allows both elements to bind to their receptors on the same cell surface at the same time. The activity element may have one or more mutations that reduce its activity, so that the prior binding to the cell via the targeting element becomes particularly important.

For example, Taylor et al. (PEDS 1-10 (2010)) and Burrill et al. (PNAS 113:5245-5250 (2016)) described a targeted form of erythropoietin that is directed to red blood cell precursors. Similarly, Cironi et al. (JBC 283:8469-8476 (2008)), WO 2008/124086, and Garcin et al. (Nature Communications 5:3016 (2014)) described targeted forms of interferon alpha and leptin that are fused to targeting elements and show activity of specific cell types in preference to other cells. A potential problem with this approach is that a given targeting element may have an undesired activity, independent of its purpose within a fusion protein. For example, in the EGF-IFNalpha fusion protein of Cironi et al., the EGF targeting element also has the activity of activating EGF receptor signaling, which is generally undesired in a cancer treatment. Similarly, different antibody elements may have inhibitory or excitatory activities when they bind to their target antigens, which may not be useful in the context of a targeted fusion protein. Therefore, there is a need in the art for targeted fusion proteins in which the properties of the antibody element are compatible with the therapeutic purpose of the fusion protein.

### SUMMARY

As described herein, the inventors have found that targeted fusion proteins using glycophorin A (GYPA) as the target molecule are particularly suspectible to certain spatial concerns. That is, mere specific targeting to GYPA is not sufficient to achieve the desired effects, rather the fusion protein must bind to precise areas of GYPA and maintain a maximum overall size in order to provide effective, on target activity.

In one aspect of any of the embodiments, described herein is a polypeptide comprising a) an anti-GYPA antibody reagent that binds the epitope of SEQ ID NO: 25, b) an erythropoietin, and c) a linker sequence separating the anti-GYPA antibody reagent and the erythropoietin.

In one aspect of any of the embodiments, described herein is a polypeptide comprising an anti-GYPA antibody reagent, a linker sequence of no more than 17 amino acids, and an activity element.

In some embodiments of any of the aspects, the anti-GYPA antibody comprises IH4 or 10F7. In some embodiments of any of the aspects, the anti-GYPA antibody reagent comprises one or more CDRs of IH4. In some embodiments of any of the aspects, the anti-GYPA antibody reagent comprises the three CDRs of IH4. In some embodiments of any of the aspects, the anti-GYPA antibody reagent comprises a VHH having the sequence of SEQ ID NO: 10 or 11. In some embodiments of any of the aspects, the anti-GYPA antibody reagent comprises one or more CDRs of an antibody reagent selected from R18, IH4, IH4v1, and Table 3. In some embodiments of any of the aspects, the anti-GYPA antibody reagent comprises the CDRs of an antibody reagent selected from R18, IH4, IH4v1, and Table 3. In some embodiments of any of the aspects, the anti-GYPA antibody reagent comprises the VH and VL sequences of an antibody reagent selected from R18, IH4, IH4v1, and Table 3. In some embodiments of any of the aspects, the anti-GYPA antibody reagent comprises an antibody reagent selected from R18, IH4, IH4v1, and Table 3. In some embodiments of any of the aspects, the antibody reagent is selected from R18, IH4, IH4v1, 2B-21, 2B-25, 2B-9, 2B-20, 2B-19, NaM70-3C10, 2B-4, B14, B89, R7, and BRIC 93. In some embodiments of any of the aspects, the antibody reagent is selected from IH4, IH4v1, 2B-9, 2B-20, 2B-19, NaM70-3C10, 2B-4, B14, B89, R7, and BRIC 93.

In some embodiments of any of the aspects, the erythropoietin comprises at least one mutation at an amino acid residue of SEQ ID NO: 16 selected from R150, A30, H32, P87, W88, P90, R53, and E55. In some embodiments of any of the aspects, the at least one mutation is R150A, A30N, H32T, P87V, W88N, P90T, R53N, or E55T.

In some embodiments of any of the aspects, the linker sequence is no more than 17 amino acids in length. In some embodiments of any of the aspects, the linker sequence is at least 5 amino acids in length. In some embodiments of any of the aspects, the linker sequence is 5-35 amino acids in length. In some embodiments of any of the aspects, the linker sequence is 5-7 amino acids in length. In some embodiments of any of the aspects, the linker sequence is 7 or fewer amino acids in length.

In one aspect of any of the embodiments, described herein is a nucleic acid encoding a polypeptide as described herein. In one aspect of any of the embodiments, described herein is a vector comprising a nucleic acid encoding a polypeptide as described herein. In one aspect of any of the embodiments, described herein is a cell comprising i) a nucleic acid encoding a polypeptide as described herein or ii) a vector comprising a nucleic acid encoding a polypeptide as described herein.

In one aspect of any of the embodiments, described herein is a method of increasing erythropoiesis comprising contacting a red blood cell with a polypeptide as described herein. In one aspect of any of the embodiments, described herein is a method of treating anemia in a subject in need thereof, the method comprising administering a polypeptide as described herein to the subject. In one aspect of any of the embodiments, described herein is a polypeptide as described herein for use in treating anemia in a subject in need thereof. In some embodiments of any of the aspects, the subject has or is diagnosed as having chronic renal failure or altitude sickness or has received chemotherapy.

### BRIEF DESCRIPTION OF THE DRAWINGS

FIGS. 1A-1B depict schematics of the design of targeted EPO plasmid constructs. The schematics are plasmid DNA constructs for the expression of Targeted EPO in a mammalian cell line. A DNA sequence for IH4v1-5aa-EPOR150A is inserted into (FIG. 1A) pSecTag2A vector for the transient expression in HEK239F cells and (FIG. 1B) pOptiVEC vector for the transient and stable expression in CHO-S and CHO-DG44 cells, respectively.

FIGS. 2A-2D depict the expression and purification of Targeted EPO proteins. IH4v1-5aa-EPOR150A proteins were produced by CHO-S cells by transient transfection. FIG. 2A demonstrates that protein expression was confirmed by Western blotting of cell culture supernatant using a His tag antibody. Proteins were purified by (FIG. 2B) nickel-IDA resins, followed by (FIG. 2C) size exclusion chromatography. FIG. 2D depicts collection fractions for the second peak (larger one) analyzed by SDS-PAGE followed by Coomassie Blue staining. Relevant molecular weights are: IH4v1-5aa-EPOR150A, 48 kDa; Legumain, 60 kDa.

FIGS. 3A-3C depict the in vitro activity of targeted EPO proteins produced by HEK293F cells. TF-1 cell proliferation is stimulated by (FIG. 3A) targeted EPO with different GYPA antibodies as targeting elements, (FIG. 3B) 10F7-EPOR150A with varying linker lengths, and (FIG. 3C) IH4v1-EPOR150A with varying linker lengths. Data shown are mean±SEM (n=3).

FIG. 4 depicts a schematic illustrating the molecular geometry of Targeted EPO on a target cell (e.g., a mature RBC or RBC precursor) IH4(v1)-5aa-EPOR150A is tethered to the target cell surface via antibody-GYPA interaction. When it binds to GYPA on a mature RBC, the linker should be short enough to prevent the binding and signaling of EPO on an EPOR on other cell types, such as vascular endothelial cells. When it binds to GYPA on a RBC precursor, the linker should be long enough to orient EPO to an EPOR on the same cell surface.

FIGS. 5A-5B depict schematics illustrating the relative positions of Targeted EPO tethered to GYPA on the mature RBC surface, illustrating the position of the GYPA epitope and EPO tethered to GYPA. FIG. 5A demonstrates that RBC-bound Targeted EPO can interact with an EPOR on a vascular endothelial cell in trans if the EPO element were allowed to extend beyond the N-terminus of GYPA as situated on the red blood cell surface. FIG. 5B demonstrates the positions of GYPA antibody binding epitopes and the linker lengths can be engineered to place the EPO element at the optimal position. The binding epitopes of 10F7 and IH4(v1) are shown. EPO fused to 10F7 is shown as darker rectangles and EPO fused to IH4(v1) is shown as lighter rectangles. The length of the linker that connects the targeting element and EPO is labelled inside the rectangles. The y-axis indicates the approximate distance from the IH4(v1) binding epitope.

FIGS. 6A-6B depict schematics of the RBC surface covered with GYPA from (FIG. 6A, left) the side and (FIG. 6B, left) the top. The average distance between the two adjacent GYPA molecules is about 140 Å and the diameter of highly glycosylated GYPA is about 15-20 Å, resulting in about 120 Å of clear space. The extracellular domain of GYPA is inherently disordered and can be assumed as being mostly linear. Its length from the membrane is estimated as 100-200 Å. Schematics of (FIG. 6A, right) Targeted EPO and (FIG. 6B, right) IgG antibody in a T-shape are shown for comparison.

FIGS. 7A-7B demonstrate that the accessibility of GYPA-bound EPO by 6×His tag-PE antibodies on the RBC surface is dependent on the GYPA binding epitope as well as the linker length. RBCs were incubated with Targeted EPO proteins, washed, and then incubated with the His tag antibodies against the peptide tag prior to flow cytometric analysis. FIGS. 7A and 7B demonstrate that the EPO element fused to 10F7 is more accessible by the antibodies than the one fused to IH4v1. The longer linkers made the EPO element more accessible to the antibodies. Data shown are mean±SEM (n=3).

FIGS. 8A-8D demonstrate that RBC clumping is mediated by bivalent antibodies against Targeted EPO and is dependent on the GYPA antibody binding epitope and the linker length. RBCs were incubated with Targeted EPO proteins, washed, and then incubated with bivalent antibodies against the peptide tag prior to flow cytometric analysis. (A, B) Clumps and (C, D) single cells were gated by forward scattering. Data shown are mean±SEM (n=3).

FIGS. 9A-9B demonstrate the pharmacodynamic effects of IH4v1-5aa-EPOR150A in mice. Human GYPA-transgenic FVB mice received a single i.p. injection of Darbepoetin, Targeted EPO, or saline. Blood samples obtained by tail-nick were analyzed by flow cytometry for (FIG. 9A) reticulocyte counts and (FIG. 9B) reticulated platelet counts. Measurements were baseline-subtracted relative to Day 0. Data shown are mean±SEM (n=5).

FIGS. 10A-10B depict graphs of the thrombotic side effects of EPO in mice. Human GYPA-transgenic FVB mice received a single i.p. injection of Darbepoetin, Targeted EPO, or saline. 24 hours later, tail transection was performed and bleeding time was measured. FIG. 10A depicts a graph of the comparison of targeted EPO proteins composed of 10F7 and IH4v1 and those containing 35aa and 5aa linkers. FIG. 10B depicts graphs of repeat experiments using IH4v1-5aa-EPOR150A gave consistent results. Box and whisker plots show median, first and third quartiles, and maximum and minimum values (n=10).

FIGS. 11A-11B depict the engineering of protein production cell lines for Targeted EPO. CHO-DG44 cells were co-transfected with the Targeted EPO construct and the plasmid encoding Cas9 and gRNA targeting the exon 4 (gRNA42) or 5 (gRNA52) of the legumain gene. FIGS. 11A-11B demonstrate that after monoclonal selection, expression levels of Targeted EPO and legumain proteins were measured by Western blotting.

FIG. 12 depicts a quantitative illustration of mechanism of targeted EPO for a 10F7-long linker-EPO(R150A) protein.

FIG. 13 depicts a quantitative illustration of mechanism of targeted EPO for an IH4-short linker-EPO(R150A) protein.

FIG. 14 depicts a sequence alignment comparing the glycophorin A sequences of humans, chimpanzees, orangutans, gorillas, bonobo chimpanzees, rhesus monkeys, cynomolgus monkeys (aka the crab-eating macaque), and the black snub-nosed monkey (Rhinopithecus bieti). Also indicated are O-linked and N-linked glycosylation sites (all in the extracellular domain, underlined), the epitopes for the anti-glycophorin antibodies R10, 10F7, and IH4, and the transmembrane and intracellular domains.

FIGS. 15A-15F. FIG. 15A depicts a schematic of the chimeric activator illustrating a targeting element and a mutated activity element fused via a flexible glycine-serine peptide linker.

FIG. 15B demonstrates that the chimeric activator binds to a target cell via the targeting element and tethers the mutated activity element around the cell surface. The increased local concentration of the mutated activity element allows for the receptor binding and signaling despite the mutation. FIG. 15C demonstrates that on a non-target cell, lacking the surface marker which the targeting element binds, mutated activity element has little or no effect due to weakened receptor binding affinity. FIG. 15D demonstrates that targeted EPO molecules also bind to mature RBCs via GPA. EPO-R is not present on these cells. The desired effect is to create a sink for Targeted EPO to extend its serum half-life.

FIG. 15E depicts a schematic of the extracellular portion GPA (blue line), showing amino acid positions (numbers), O-linked glycosylation sites (small arrows), the N-linked glycosylation site (large arrow), and the positions of the 10F7 and IH4 epitopes. FIG. 15F depicts schematics of the 10F7-35-EPO(R150A) and IH4-5-EPO(R150A) forms of Targeted EPO, showing sizes in Angstroms (Å) based on typical scFv and nanobody sizes, an extended conformation of the Gly/Ser linkers, and solved structures of EPO.

FIGS. 16A-16D demonstrate the in vitro and in vivo activity of different forms of Targeted EPO. FIG. 16A depicts atypical TF-1 cell proliferation assay comparing the stimulation of proliferation by “wild-type” EPO (including C-terminal tags), the EPO(R150A) mutant protein, and the IH4-35-EPO(R150A) fusion protein. The EC50 is defined through a 4-parameter fit. FIG. 16B depicts a table summarizing the ability of several different forms of Targeted EPO and control proteins to promote proliferation of TF-1 cells in vitro. FIG. 16C depicts an in vivo test of the ability of IH4-5-EPO(R150A) to specifically promote RBC production and not platelet production, using transgenic mice that express human GPA on their RBCs.⁷ The results indicate that IH4-5-EPO(R150A) and diverse other forms of Targeted EPO show enhanced activity of the EPO(R150A) element independently of the mode of attachment of EPO to the GPA-binding antibody element.

FIGS. 17A-17B depict a reduction in membrane fluidity by 10F7 but not IH4, measured by fluorescence recovery after photobleaching (FRAP). In this technique, RBC membranes are stained with a lipid-soluble dye, a portion (1×1 μm) of the membrane is bleached with a laser, and the diffusion of the dye back into the bleached region is measured. FIG. 17A depicts serial snapshots of RBCs treated with antibody fragments during FRAP experiments. Each recording was made at a rate of 8 frames per second for 10 seconds. FIG. 17B depicts mobile lipid fraction of 8-9 RBCs (Experiment 1) or 16-20 RBCs (Experiment 2) treated with buffer, 10F7, and IH4. Lines indicate mean and 95% confidence interval. The distribution of individual cell responses is typical for these assays.^{29, 30}

FIGS. 18A-18F demonstrate that targeted EPO fusion proteins with short linkers and membrane-proximal GPA binding epitopes reduce fusion protein accessibility and potential for cell crosslinking. (FIGS. 18A, 18B) Forms of Targeted EPO containing either 10F7 or IH4 and varying linker lengths were incubated with human RBCs, and stained with a divalent phycoerythrin-labeled anti-His₆ antibody. (FIG. 18A) The accessibility of the EPO element by anti-His₆-PE antibody is shown as mode fluorescence intensity (PE). (FIG. 18B) The crosslinking of RBCs mediated by divalent anti-His₆ antibody is shown as forward scattering (particle size). The signal greater than the cutoff (dotted line) is consistent with adhesion of RBCs. (FIGS. 18C-18F) Human RBCs treated with Targeted EPO variants were incubated with (FIGS. 18C, 18D) A2780 or (FIGS. 18E, 18F) MCF-7 cells, both of which express EPO-R. (FIGS. 18C, 18E) Phase-contrast images of RBC rosetting around EPO-R-bearing cells. 200× magnification. (FIGS. 18D, 18F) The frequency of rosette formation was quantitated by tumor cells bound by 3 or more RBCs, shown as a percentage of total tumor cells. In FIGS. 18A, 18B, 18D, and 18E, the first four line/bars (from top/left respectively) are 10F7-EPO(R150A), the next four are IH4-EPO(R150A), and the last (if present) is PBS.

FIGS. 19A-19D demonstrate that shortened tail bleeding time in mice treated with non-targeted forms of EPO and with 10F7-EPO(R150A) but not with IH4-EPO(R150A). (FIG. 19A) Experimental approach. Mice received a single ip injection of darbepoetin or epoetin alpha (50 pmol; 1.8 mcg), 10F7-EPO(R150A) (125 pmol; 9 mcg); IH4-EPO(R150A) (40 pmol; 2 mcg), or vehicle. 24 hours later, tail transection was performed and bleeding times were recorded (See Methods). (FIG. 19B) Three experiments showing bleeding time relative to the saline control in huGPA-transgenic mice treated with saline, darbepoetin, IH4-5-EPO(R150A). Data shown are median with interquartile range. (FIG. 19C) A Kaplan-Meier plot for a representative data set (Experiment 1) from the data shown in (FIG. 19B). (FIG. 19D) Bleeding times after treating huGPA-transgenic or non-transgenic mice with non-targeted forms of EPO and targeted EPO variants. In each case, the bleeding times are normalized to the median of a vehicle control performed on the same day, to account for day-to-day variability.

FIGS. 20A-20C provide a summary of bleeding times after treating huGPA-transgenic or non-transgenic mice with various forms of non-targeted and targeted EPO. Mice received a single ip injection of darbepoetin or epoetin alfa (50 pmol; 1.8 μg), 10F7-EPO(R150A) (125 pmol; 9 μg), IH4-5-EPO(R150A) (40 pmol; 2 μg), or vehicle on day 0, and bleeding times were measured on day 1. In experiment 9 (italics, *), mice received two ip injections of darbepoetin (50 pmol; 1.8 μg), IH4-35-EPO(R150A) (180 pmol; 9 μg or 20 pmol; 1 μg), or vehicle on days 0 and 3, and bleeding times were measured on day 4. (FIG. 20A) Raw data from all 13 bleeding time experiments. Data represent median with interquartile range. (FIG. 20B) Description of each experiment shown in (FIG. 20A). (FIG. 20C) Bleeding times were normalized to the median of a vehicle control performed on the same day, to account for day-to-day variability. The normalized values were combined across experiments and Mann-Whitney test was performed. *p<0.1; **p<0.05; ***p<0.01; ns: not significant.

FIG. 21 depict the ability of a form of Targeted EPO, R18-5-EPO(R150A), to promote RBC production in human GPA-transgenic mice. Compared to IH4-5-EPO(R150A), a higher dose of R18-5-EPO(R150A) is needed to induce comparable increase in reticulocytes.

FIGS. 22A-22B depict the protein sequence alignments of IH4 and other nanobodies. (FIG. 22A) The original IH4 sequence (IH4*) was modified to match the consensus sequence. IH4* (blue) indicates the original protein sequence of the IH4 nanobody from the U.S. Pat. No. 9,879,090.¹ Phe80 in the framework region 3 of IH4* is mutated to tyrosine (green highlight), and a threonine residue is inserted between Gly117 and Gln118 in the framework region 4 of IH4* (green highlight). The resulting sequence is shown as IH4 (orange, bold). Each dot indicates a position for a single amino acid residue. Numbers indicate amino acid positions every 10 residues. Complementarity-determining regions (CDRs) are shown with dots highlighted in gray and underlined sequences. Sequences between CDRs are framework regions. (FIG. 22B) PDB ID's and brief descriptions of six nanobodies used in sequence alignments.

FIGS. 23A-23B depict phase-contrast images of RBC rosetting around EPO-R-bearing cells. Human RBCs treated with Targeted EPO variants were incubated with (FIG. 23A) A2780 or (FIG. 23B) MCF-7 cells. 200× magnification.

FIGS. 24A-24C depict a geometric model of the RBC surface. There are 800,000 GPA monomers densely packed on the RBC surface, whose area is about 140 μm². The average distance between GPA molecules is about 130-190 Angstroms (Å). (FIG. 24A) When all GPA molecules are monomeric, the average distance between the two adjacent monomers is about 132 Å. (FIG. 24B) When all GPA molecules dimerize, the average distance between the two adjacent dimers is about 187 Å. O- and N-linked glycans extend 20 Å from the peptide backbone. About 90-100 Å between GPA molecules can be freely accessed by external molecules. (FIG. 24C) Summary of relevant numbers and equations to estimate the density and accessibility of GPA molecules on the RBC surface.

FIGS. 25A-25F. FIG. 25A depicts the protein sequence organization of a Targeted EPO chimeric activator. (FIG. 25B) The chimeric activator binds to a target cell via the targeting element and tethers the mutated activity element to the cell surface, so that the mutated activity element binds to its receptor despite the mutation. (FIG. 25C) On a non-target cell, lacking the surface marker to which the targeting element binds, the mutated activity element has little effect. (FIG. 25D) Targeted EPO molecules also bind to mature RBCs via GPA, creating a sink for a Targeted EPO to extend its plasma half-life. EPO-R is not present on these cells. (FIG. 25E) The extracellular portion of GPA (line), showing amino acid positions (numbers), O-linked (small arrows) and N-linked (large arrow) glycosylation sites, and the positions of the 10F7 and IH4 epitopes. (FIG. 25F) The 10F7-35-EPO(R150A) and IH4-5-EPO(R150A) forms of Targeted EPO, showing sizes in Angstroms (Å) based on typical scFv and nanobody sizes, an extended conformation of the Gly/Ser linkers, and solved structures of EPO. Some images were created with BioRender.

FIGS. 26A-26D depict the in vitro and in vivo activity of different forms of Targeted EPO. (FIG. 26A) A typical TF-1 cell proliferation assay comparing the stimulation of proliferation by “wild-type” EPO (including C-terminal tags), the EPO(R150A) mutant protein, and the IH4-35-EPO(R150A) fusion protein. The effective concentration (EC50) is calculated by a 4-parameter fit. (FIG. 26B) A table summarizing the ability of different forms of Targeted EPO and control proteins to promote proliferation of TF-1 cells in vitro. (FIGS. 26C-26D) An in vivo test of the ability of IH4-5-EPO(R150A) to specifically promote RBC production and not platelet production, using transgenic mice that express human GPA on their RBCs.⁴ The results indicate that IH4-5-EPO(R150A) and diverse other forms of Targeted EPO show enhanced activity of the EPO(R150A) element independently of the mode of attachment of EPO to the GPA-binding antibody element. Data represent mean±SEM.

FIGS. 27A-27B depict reduction in membrane fluidity by 10F7 but not IH4, measured by FRAP. RBC membranes are stained with a lipid-soluble dye, a portion of the membrane is bleached with a laser, and diffusion of the dye into the bleached region is measured. (FIG. 27A) Serial snapshots of RBCs treated with antibody fragments during FRAP experiments. Each recording was made at 8 frames per second for 10 seconds. (FIG. 27B) Mobile lipid fraction of 8-9 RBCs (Experiment 1) or 16-20 RBCs (Experiment 2) treated with buffer, 10F7, or IH4. When the data from these experiments were normalized to the buffer controls in the same experiments and then combined, the difference between 10F7 and buffer (p=0.0648) or 10F7 and IH4 (p=0.0463) was significant (Mann-Whitney test). Lines indicate mean and 95% confidence interval. The distribution of individual cell responses is typical for these assays.¹⁶

FIGS. 28A-28F demonstrate that targeted EPO fusion proteins with short linkers and membrane-proximal GPA binding epitopes reduce EPO accessibility and cell crosslinking. (FIGS. 28A, 28B) Targeted EPO's containing either 10F7 or IH4 and varying linker lengths were incubated with RBCs, and stained with a PE-labeled anti-His₆ antibody. (FIG. 28A) The accessibility of the EPO element by anti-His₆-PE antibody is shown as mode fluorescence intensity (PE). (FIG. 28B) The crosslinking of RBCs mediated by divalent anti-His₆ antibody is shown as forward scattering (particle size). The signal greater than the cutoff (dotted line) indicates adhesion of RBCs. (FIGS. 28C-28E) RBCs treated with Targeted EPO variants were incubated with A2780 or MCF-7, both of which express EPO-R. (FIG. 28C) Phase-contrast images of RBC rosetting around EPO-R-bearing cells. 200× magnification. (FIGS. 28D, 28E) The frequency of rosette formation was scored as tumor cells bound by 3 or more RBCs, shown as a percentage of total tumor cells. (FIG. 28F) Relative geometry and distances of GPA and the bound Targeted EPO on the RBC surface. Data represent mean±SEM. In FIGS. 28A, 28B, 28D, and 28E, the first four line/bars (from top/left respectively) are 10F7-EPO(R150A), the next four are IH4-EPO(R150A), and the last (if present) is PBS.

FIGS. 29A-29C depict shortened tail bleeding time in mice treated with a non-targeted EPO but not with IH4-5-EPO(R150A). (FIG. 29A) Experimental approach. Mice received a single ip injection of darbepoetin (50 pmol; 1.8 μg), IH4-5-EPO(R150A) (40 pmol; 2 μg), or vehicle. 24 hours later, tail transection was performed and bleeding times were recorded (See Methods). (FIG. 29B) Three experiments showing bleeding times relative to the vehicle control in huGPA-transgenic mice treated with vehicle, darbepoetin, or IH4-5-EPO(R150A). Data represent median with interquartile range. (FIG. 29C) A Kaplan-Meier plot for Experiment 1 from the data shown in (FIG. 29B).

FIG. 30 depicts a diagram of the fusion protein action.

FIGS. 31A-31C depict a summary of bleeding times after treating huGPA-transgenic or non-transgenic mice with various forms of non-targeted and targeted EPO. Mice received a single ip injection of darbepoetin or epoetin alfa (50 pmol; 1.8 μg), 10F7-EPO(R150A) (125 pmol; 9 μg), IH4-5-EPO(R150A) (40 pmol; 2 μg), or vehicle on day 0, and bleeding times were measured on day 1. In experiment 9 (italics, *), mice received two ip injections of darbepoetin (50 pmol; 1.8 μg), IH4-35-EPO(R150A) (180 pmol; 9 μg or 20 pmol; 1 μg), or vehicle on days 0 and 3, and bleeding times were measured on day 4. (FIG. 31A) Raw data from all 13 bleeding time experiments. Data represent median with interquartile range. (FIG. 31B) Description of each experiment shown in (FIG. 31A). (FIG. 31C) Bleeding times were normalized to the median of a vehicle control performed on the same day, to account for day-to-day variability. The normalized values were combined across experiments and Mann-Whitney test was performed. *p<0.1; **p<0.05; ***p<0.01; ns: not significant.

FIGS. 32A-32B demonstrate that targeted EPO fusion proteins preferentially bind RBCs in a mixed population of RBCs and A2780 cells. (FIG. 32A) Flow cytometric analyses showing the distribution of cells by size (FSC) and the binding of fusion proteins detected by the anti-His6-PE antibody (PE). Q1: RBC bound by fusion proteins; Q2: A2780 bound by fusion proteins; Q3: unbound A2780; Q4: unbound RBC. (FIG. 32B) Quantification of PE-positive population as a percentage of the parent population (RBC or A2780). Data represent mean±SEM. First series is RBC and the second series is A2780.

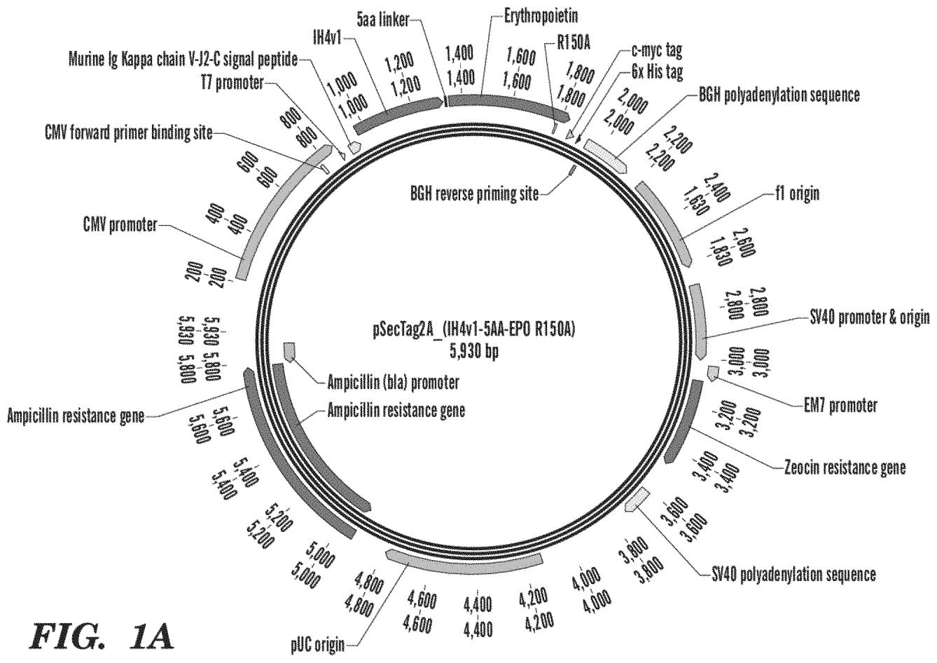

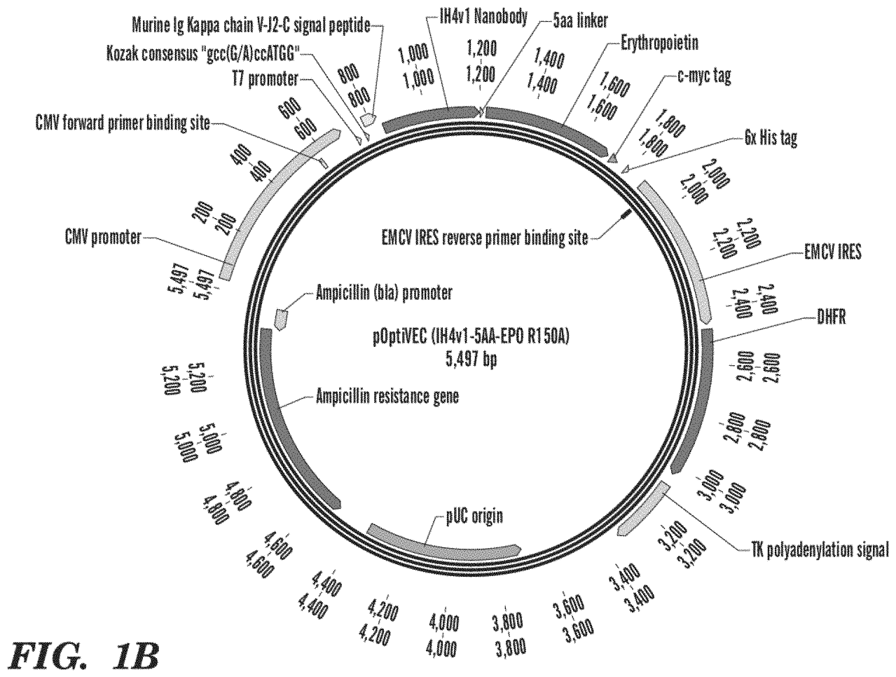

### DETAILED DESCRIPTION

As described herein, the inventors have found that specific targeting elements and specific linker sizes can provide an engineered erythropoietin which avoids the harmful side effects of existing EPO therapeutics. The compositions and methods described herein relate to forms of erythropoietin (“EPO”) that are targeted to red blood cell precursors and away from EPO receptor-bearing cells on other cell types that may lead to side effects. It is noted that these finding are particularly suprising as the structure of GYPA was not previously known well enough to predict how to achive such spatial organization of the compositions described herein, and whether they could retain EPO activity when subject to the spatial limitations described herein.

EPO has been considered to be an ‘anemia hormone’ whose primary purpose is to promote formation of red blood cells. EPO is produced primarily by the kidney. Kidney failure patients produce little or no EPO from the kidney, and have reduced levels of red blood cells as a result. (A small amount of EPO is also produced by the liver.) In fact, EPO is a pleiotropic hormone that signals in diverse cell types. EPO promotes the differentiation of RBC precursors, and recombinant EPO is used to treat anemia due to chronic kidney disease and myelosuppressive cancer chemotherapy; however, EPO also signals on megakaryocytes, capillary endothelial cells, and tumor cells. EPO action on these cells may promote the thrombosis and tumor progression, documented in clinical trials, that have led to “black box” warnings on EPO-based products.

Burrill et al. (PNAS 113:5245-5250 (2016)) constructed a targeted form of EPO, 10F7-linker-EPO(R150A), with the following characteristics:
1. The EPO molecule was mutated to weaken its affinity for EPO-R, using the mutation Arg150Ala, which reduces binding by about 12-fold;
2. The avidity for RBC precursors was rescued by tethering to a targeting element, an scFv form of the 10F7 antibody, which specifically binds the human RBC marker glycophorin A (huGYPA);
3. The linker between the 10F7 scFv and EPO was about 35 amino acids long, consisting primarily of glycine and serine.

Human Glycophorin A (huGYPA) is a very abundant protein found on the surface of red blood cells (RBCs), with about 800,000 copies per cell. huGYPA is also present on late RBC precursors, with up to 50,000 copies per cell. The copy number of EPO receptor on late RBC precursors is about 100-1,000.

The concept of the “Targeted EPO” type of fusion protein is that the antibody element that binds to huGYPA first, and then the EPO element binds to its receptor and activates signal transduction. The EPO element is able to bind to its receptor, in spite of the weakening mutation, because the prior binding of the antibody element to huGYPA places the EPO protein element in a very high local concentration around the cell surface.

Described herein are optimal antibody elements and linkers for construction of an optimal form of Targeted EPO. This improved targeted EPO provides a suprising lack of negative side effects without compromising therapeutic efficacy. It is described herein that different anti-glycophorin antibody elements vary widely in their suitability for use in a Targeted EPO fusion protein. In particular, the antibody elements vary in their ability to be expressed, the strength of their binding to huGYPA, and their tendency to modulate an apparent inflammatory signal transduction pathway mediated by the interaction of huGYPA and a second RBC membrane protein, Band 3. The following antibody elements were used:

| | | |
|---|---|---|
| 1. | 10F7 | (SEQ ID NO: 46) |
| | | >ANC33496.2 10F7-linker-EPO, partial [synthetic construct] |
| | | Heavy: |
| | | QVKLQQSGAELVKPGASVKLSCKASGYTFNSYFMHWMKQRPVQGLEWIGM |
| | | IRPNGGTTDYNEKFKNKATLTVDKSSNTAYMQLNSLTSGDSAVYYCARWEG |
| | | SYYALDYWGQGTTVTVS |
| | | Linker: SGGGGSGGGGSSGGGGSS |
| | | Light: DIELTQSPAIMSATLGEKVTMTCRASSNVKYMYWYQQKSGASPKLWIY |
| | | YTSNLASGVPGRFSGSGSGTSYSLTISSVEAEDAATYYCQQFTSSPYTFGGGT |
| | | KLEIK |
| 2. | 1C3 | Heavy: (SEQ ID NO: 47) |
| | | EVRLLESGGGPVQPGGSLKLSCAASGFDFSRYWMNWVRRAPGKGLEWIGEI |
| | | N |
| | | QQSSTINYSPPLKDKFIISRDNAKSTLYLQMNKVRSEDTALYYCARLSLTAAG |
| | | FAYWGQGTLVTVS |
| | | Light: (SEQ ID NO: 48) |
| | | DIVMSQSPSSLAVSVGEKVSMSCKSSQSLFNSRTRKNYLTWYQQKPGQSPKP |
| | | LIYWASTRESGVPDRFTG |
| | | SGSGTDFTLTISSVQAEDLADYYCKQSYNLRTFGGGTKLEIK |
| 3. | R18 | Heavy chain (SEQ ID NO: 49) |
| | | QVKLQQSGGGLVQPGGSLKLSCAASGFTFSSYG |
| | | MSWFRQTPDKRLELVAIINSNGGTTYYPDSVKGRFTISRDNAKNTLYLQMSS |
| | | LKSEDTAMYYCARGGGRWLLDYWGQGTTVTVSS |
| | | Light chain (SEQ ID NO: 50) |
| | | DIELTQSPSSLAVSAGEKVTMSCKSSQSVLYSSNQKNYLAWYQQKPGQSPKL |
| | | LIYWASTRESGVPDRFTGSGSGTDFTLTISSVQAEDLAVYYCHQYLSSSTFGG |
| | | GTKLEIK |
| 4. | IH4 | (SEQ ID NO: 10) |
| 5. | IH4v1 | (SEQ ID NO: 11) |

The data and insights of the invention indicate that the antibody elements IH4 and IH4v1 provide the best results in the Targeted EPO molecules described herein. A particular insight described herein is that certain antibodies to glycophorin A can cause an inflammatory response by red blood cells, which includes stiffening of the RBC; reduction in membrane fluidity; release of reactive oxygen species such as hydrogen peroxide; and release of ATP, which is a mediator of inflammation and pain when it is outside of cells. According to the methods and compositions described herein, the criteria for choosing a glycophorin-binding antibody element include its binding strength, the ability of the antibody element to be expressed from cultured mammalian cells, the epitope on glycophorin A that is bound, and the propensity of the antibody element, especially as a monomeric element, to induce glycophorin A-mediated inflammation as defined herein. With respect to these issues, the antibody elements have the following characteristics.

TABLE 1

| | | | | | | Sequence | CDRs |
|---|---|---|---|---|---|---|---|
| Antibody | | Binding | | | | (SEQ ID | (SEQ ID |
| element | Format | strength | Expression | Epitope | Inflammation | NO) | NO) |
| 10F7 | scFv | 100 nM | Good | 27-38 | Strong | | |
| 1C3 | scFv | 30 nM | Poor | ? | ? | | |
| R18 | scFv, Fab | 400 nM | Good | 49-52 | Weak/none | 49, 50 | |
| IH4 | Nanobody | 30 nM | OK | 52-55 | Weak/none | 10 | 27-29 |
| IH4v1 | Nanobody | 30 nM | Good | 52-55 | Weak/none | 11 | 27-29 |

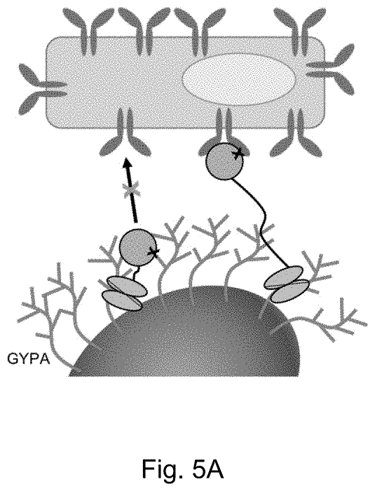

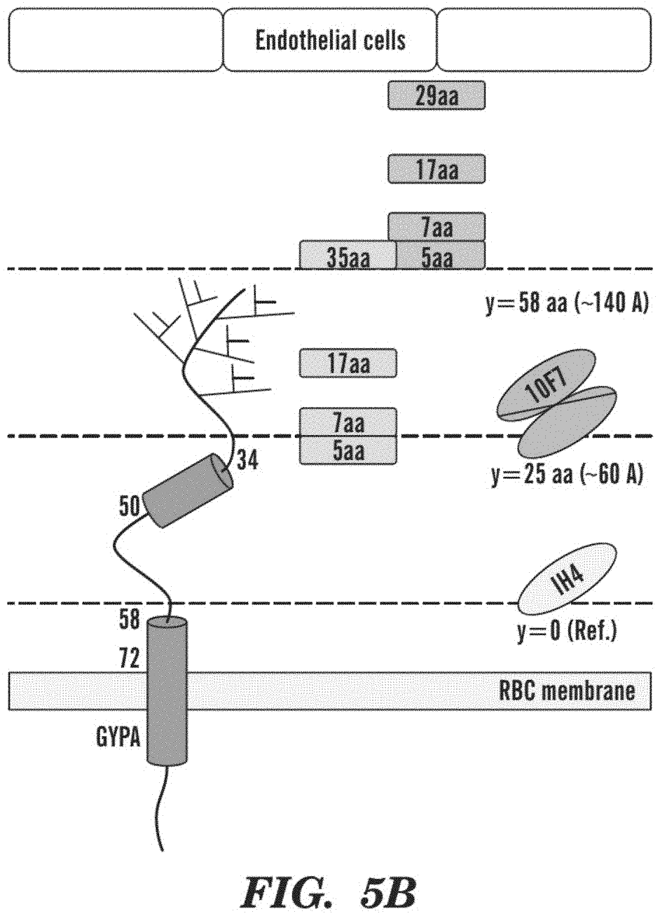

The position of the glycophorin A epitope to which an antibody element binds can have a significant effect on the usefulness of a given form of a Targeted EPO, especially when considered in combination with the length of the linker. Specifically, it is generally undesirable for the position of the binding epitope, in combination with a linker of a certain length, to allow the EPO element to extend beyond the N-terminus of the glycophorin A protein as situated on the red blood cell surface. FIGS. 5A-5B illustrate this principle.

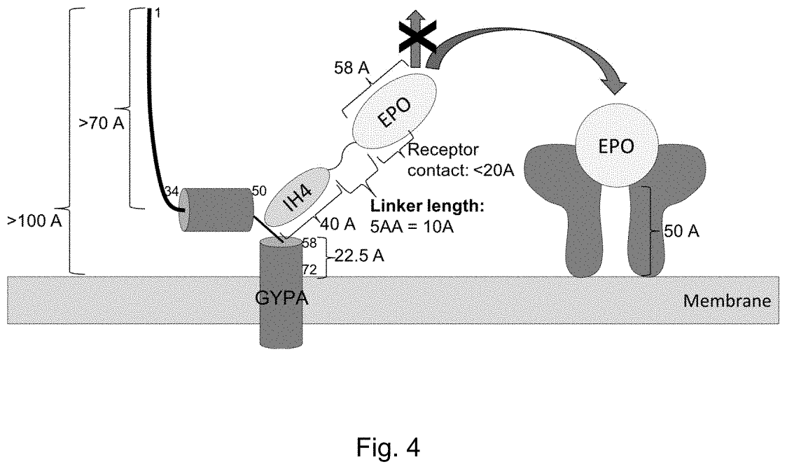

Without wishing to be bound by theory, it contemplated herein that extension of the EPO element beyond the membrane-distal N-terminus of glycophorin A may be deleterious because such EPO elements may interact with EPO receptors on vascular endothelial cells. The result would be that a red blood cell may stick to the wall of a capillary or a larger blood vessel. It is further contemplated herein that the extracellular domain of glycophorin A extends directly away from the cell membrane in a roughly linear manner, with the N-terminus being most distal and the Gly72 being most membrane proximal (see, e.g., FIG. 4).

The extracellular portion of glycophorin A is thought to be primarily an intrinsically disordered protein. The first 25 amino acids of the mature glycophorin A are highly and variably 0-glycosylated and there is an N-linked glycosylation site at position 26. The high level of glycosylation likely prevents this region from folding into a typical, compact protein structure. The amino acids from 27 to 69 have a high ratio of charged and hydrophilic amino acids compared to hydrophobic amino acids, and include several O-linked glycosylation sites. These characteristics are also consistent with the idea that much of this region is intrinsically disordered.

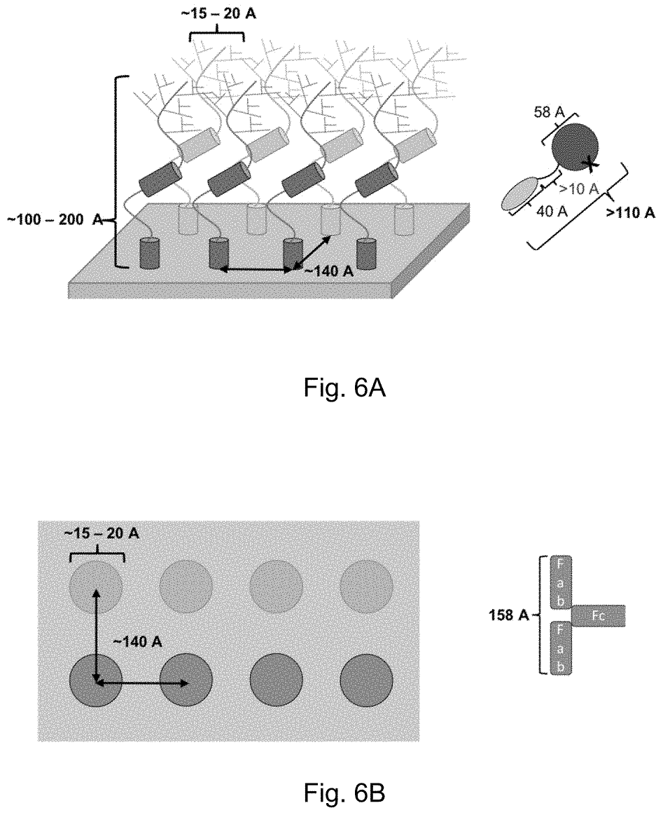

The density of glycophorin A on a red blood cell membrane is so high that this protein likely shields red blood cells from closely approaching each other or other cells. There are about 800,000 copies of glycophorin A on a red blood cell surface, and the surface area of a red blood cell is about 150 square microns, such that the average distance between glycophorin A monomers would be about 14 nanometers or 140 Angstroms. The distance of the glycophorin A N-terminus from the cell membrane may be as great as about 200 Angstroms. Moreover, the oligosaccharides at the N-terminus of glycophorin A are likely to extend laterally by 15-20 Angstroms from the peptide chain. FIGS. 6A-6B schematically present a top-down view of a red blood cell surface covered with glycophorin A.

Glycophorin A extracellular domain. O-linked glycosylation sites are shown in caps.

```
                10 20 30 40 50 60
70
. . |. . |. . |. . |. . |. . |. . |. . |. . |. . |. . |. . |. . |. .
|. . |
1STTevamhTSTSssvTkSyiSsgTNdthkrdTyaaTprahevSeiSvrTvyppeeetgervqlahhf
sepeitliifgvmagvi...
                                                    <-10F7 site> <IH4>
<trans-membrane>
```

For an extended amino acid chain, such as a beta strand, the length is about 3 Angstroms per amino acid. This means that the IH4 binding site on glycophorin A may be about 60 Angstroms closer to the cell membrane than the 10F7 binding site. The distance from the antigen binding site of an scFv to the C-terminus of the VH domain (where the linker is attached) is about 32 Angstroms. Thus, if the scFv is angled away from the membrane, then this will increase the average distance of the EPO element from the red blood cell membrane. A fusion protein such as 10F7-(Gly+Ser)₃₉-EPO, when bound to the 10F7 site, likely allows the EPO element to extend beyond the N-terminus of glycophorin A. The binding site of 10F7 on glycophorin A is roughly 33 amino acids from the N-terminus, which is less than the length of the linker attaching 10F7 to EPO. Comparing the molecule IH4-(Gly+Ser)₅-EPO with 10F7-(Gly+Ser)₃₉-EPO, the IH4 epitope is about 20 amino acids farther from the glycophorin A N-terminus; and the linker in IH4-(Gly+Ser)₅-EPO is much shorter, which additionally minimizes the chance that the EPO element is ever membrane-distal to the glycophorin A N-terminus.

Thus, for the IH4-(Gly+Ser)₅-EPO molecule, the EPO element will not be exposed membrane-distal to the N-terminus of glycophorin A, and will not cause side effects that might have resulted from EPO interacting with EPO receptors on the vascular lining while it is tethered to red blood cells. This specific example illustrates the general point that the a form of Targeted EPO which has an antibody element that binds to a membrane-proximal region of glycophorin A, and has a short linker between the antibody element and the EPO element; is least likely to cause side effects, provided that such a molecule is still able to activate EPO receptors in a glycophorin-dependent manner.

Accordingly, in some embodiments of any of the aspects, a polypeptide described herein, e.g., a Targeted EPO satisfies the equation:

(Linker Length)+(Epitope center distance from GYPA's Glu72)<50

To precisely define these parameters, the linker length is defined as the number of amino acids before the Alanine in the EPO sequence Ala-Pro-Pro . . . and after the Lysine in the sequence . . . Lys-Leu-Glu-Ile-Lys for a fusion to a VL region; after the Serine in the sequence . . . Gln-Val-Thr-Val-Ser in a nanobody VH; or the second serine in the sequence Val-Thr-Val-Ser-Ser for a VH region. The epitope center is defined as the central amino acid in a linear glycophorin epitope, if the epitope consists of an odd number of bases, or the center-right amino acid if the epitope has an even number of bases. Because glycophorin A is an intrinsically disordered protein, its antibody epitopes are linear peptides.

In some embodiments of any of the aspects, a polypeptide described herein, e.g., a Targeted EPO satisfies the equation:

(Linker Length)+(Epitope center distance from Glu72)<80,

In some embodiments of any of the aspects, a polypeptide described herein, e.g., a Targeted EPO satisfies one of the equations that define an “RBC Membrane Distance Metric”:

(Linker Length)+(Epitope center distance from Glu72)<60,

(Linker Length)+(Epitope center distance from Glu72)<40,

(Linker Length)+(Epitope center distance from Glu72)<30, or

(Linker Length)+(Epitope center distance from Glu72)<25.

By way of non-limiting illustration, the value of (Linker Length)+(Epitope center distance from Glu72) for 10F7-(Gly+Ser)₃₉-EPO is 78, while the value of (Linker Length)+(Epitope center distance from Glu72) for IH4-(Gly+Ser)₅-EPO is 23. Specifically, the epitope center of the 10F7 V regions is at about amino acid 33, so (72-33)+39=78. The epitope center of IH4 is at about amino acid 54, so (72-54)+5=23.

The net effect of the short linker in combination with an epitope that is close to the cell surface is to position the EPO element closer to the red blood cell surface than the N-terminus of glycophorin A. The density of glycophorin A on circulating red blood cells is so high that, for a Targeted EPO with an RBC Membrane Distance Metric of less than 25, the glycophorin A will prevent the EPO element from acting on EPO receptors on other cells.

Accordingly, in one aspect of any of the embodiments, described herein is polypeptide comprising a) an anti-GYPA antibody reagent that specifically binds the extracellular domain of GYPA C-terminal of residue 34 of GYPA and b) an erythropoietin. In one aspect of any of the embodiments, described herein is polypeptide comprising a) an anti-GYPA antibody reagent that specifically binds the extracellular domain of GYPA C-terminal of residue 34 of GYPA, b) an erythropoietin, and c) a linker separating the anti-GYPA antibody reagent and the erythropoietin. In one aspect of any of the embodiments, described herein is polypeptide comprising a) an anti-GYPA antibody reagent that specifically binds the extracellular domain of GYPA C-terminal of residue 34 of GYPA, b) an erythropoietin, and c) a polypeptide linker separating the anti-GYPA antibody reagent and the erythropoietin. In some embodiments of any of the aspects, the epitope of the anti-GYPA antibody reagent is entirely C-terminal of residue 34 of GYPA. As explained elsewhere herein, see, e.g., FIG. 4, binding of the anti-GYPA antibody reagent less than about 100 A from the cell surface can restrict the activity of the Targeted EPO reagent to a single cell, avoiding inflammatory side effects. As depicted in FIG. 4, the extracellular domain located between residues 34 and 72 is located within 100 A of the cell surface. Anti-GYPA antibodies which bind outside of this region of GYPA will not display the desired characteristics of the Targeted EPO reagents described herein (see, e.g., Table 1).

In some embodiments of any of the aspects, the polypeptide comprises an anti-GYPA antibody reagent that specifically binds the extracellular domain of GYPA between resides 34 and 72 of GYPA (e.g., SEQ ID NO: 26). Binding between two residues refers to the epitope of the antibody being located entirely between those residues (inclusive of those residues). An epitope located only partially within a specified range of residues is not considered to be located between those residues. In some embodiments of any of the aspects, the epitope of the anti-GYPA antibody reagent is located between resides 34 and 72 of GYPA (e.g., SEQ ID NO: 26). In some embodiments, it is desired that the anti-GYPA antibody reagent binds within 22-100 A of the cell surface, but binds to structures other than the alpha helices (depicted as a cylinder between residues 34 and 50 of GYPA in FIG. 4) of the extracellular domain of GYPA. Accordingly, in some embodiments of any of the aspects, the polypeptide comprises an anti-GYPA antibody reagent that specifically binds the extracellular domain of GYPA between resides 49 and 72 of GYPA (e.g., SEQ ID NO: 26). In some embodiments of any of the aspects, the epitope of the anti-GYPA antibody reagent is located between resides 49 and 72 of GYPA (e.g., SEQ ID NO: 26).

In some embodiments, it is desired that the anti-GYPA antibody reagent binds within 22-100 A of the cell surface, but binds to structures other than the alpha helix(ces) of the extracellular domain of GYPA. Accordingly, in some embodiments of any of the aspects, the polypeptide comprises an anti-GYPA antibody reagent that specifically binds the extracellular domain of GYPA between resides 49 and 58 of GYPA (e.g., SEQ ID NO: 26). In some embodiments of any of the aspects, the epitope of the anti-GYPA antibody reagent is located between resides 49 and 58 of GYPA (e.g., SEQ ID NO: 26).

In some embodiments of any of the aspects, the polypeptide comprises an anti-GYPA antibody reagent that specifically binds the extracellular domain of GYPA between resides 49 and 55 of GYPA (e.g., SEQ ID NO: 26). In some embodiments of any of the aspects, the epitope of the anti-GYPA antibody reagent is located between resides 49 and 55 of GYPA (e.g., SEQ ID NO: 26).

Antibody reagents are known and provided herein which bind to the specified epitopes/regions of GYPA. For instance, Table 1 describes the epitopes of R18, 1H4, and IH4v1 which meet the specified epiptope limitations. In some embodiments of any of the aspects, the polypeptide comprises an anti-GYPA antibody reagent comprising the CDRs of R18, 1H4, or IH4v1. In some embodiments of any of the aspects, the polypeptide comprises an anti-GYPA antibody reagent comprising the VL and VH sequences of an antibody reagent selected from R18, 1H4, or IH4v1. In some embodiments of any of the aspects, the polypeptide comprises an anti-GYPA antibody reagent comprising R18, 1H4, or IH4v1.

Additional exemplary anti-GYPA antibody reagents known in the art, along with their binding specificity for GYPA are provided in Table 3.

TABLE 3
(all references mentioned in this table are incorporated
by reference herein in their entireties)

| Antibody | Epitope | See, e.g., | Commerical Source |
|---|---|---|---|
| BRIC 116 | Includes residues 35-36 | Gardner et al. | |
| | | Immunology 68: | |
| | | 283-289 (1989) | |
| R 10 | Includes residues 35-37 | Gardner et al. | |
| | | Immunology 68: | |
| | | 283-289 (1989) | |
| | | Anstee Eur. J. Immunol | |
| | | 12 (1982) | |
| Mab 158 | Within residues 35-40 | Anstee et al. Journal of | |
| | | Immunogenetics | |
| | | 17: 301-308 (1990) | |
| OSK4-1 | Residues 35-45 | Rasamoelisolo et al. | |
| | (AATPRAHEVSE | Vox Sang 72: 185-191 | |
| | (SEQ ID NO: 33)) | (1997) | |
| | | Wasinowska et al. Mol | |
| | | Immunol 29: 783-791 | |
| | | (1992) | |
| GPA 33 | Within residues 35-45 | Rasamoelisolo et al. | |
| | (AATPRAHEVSE | Vox Sang 72: 185-191 | |
| | (SEQ ID NO: 33)) | (1997) | |
| GPA 105 | Within residues 35-45 | Rasamoelisolo et al. | |
| | (AATPRAHEVSE | Vox Sang 72: 185-191 | |
| | (SEQ ID NO: 33)) | (1997) | |
| BRIC 117 | Includes residues 36-38 | Gardner et al. | |
| | | Immunology 68: | |
| | | 283-289 (1989) | |
| A88-A/F9 | Within residues 36-45 | Karsten et al. | |
| | (ATPRAHEVSE (SEQ | International | |
| | ID NO: 34)) | Immunopharmacology | |
| | | 10: 1354-1360 (2010) | |
| A88-D/C7 | Within residues 36-45 | Karsten et al. | |
| | (ATPRAHEVSE (SEQ | International | |
| | ID NO: 34)) | Immunopharmacology | |
| | | 10: 1354-1360 (2010) | |
| A88-E/H2 | Within residues 36-45 | Karsten et al. | |
| | (ATPRAHEVSE (SEQ | International | |
| | ID NO: 34)) | Immunopharmacology | |
| | | 10: 1354-1360 (2010) | |
| A96-D/A7 | Within residues 36-45 | Karsten et al. | |
| | (ATPRAHEVSE (SEQ | International | |
| | ID NO: 34)) | Immunopharmacology | |
| | | 10: 1354-1360 (2010) | |
| A96-E/F7 | Within residues 36-45 | Karsten et al. | |
| | (ATPRAHEVSE (SEQ | International | |
| | ID NO: 34)) | Immunopharmacology | |
| | | 10: 1354-1360 (2010) | |
| BRIC 119 | Includes residues 37-39 | Gardner et al. | |
| | | Immunology 68: | |
| | | 283-289 (1989) | |
| 2B-18 | Residues 38-42 | Wasinowska et al. TCB | |
| | (PRAHE (SEQ ID NO | 1: 73-75 (1997) | |
| | 35)) | | |
| 2B-12 | Residues 38-43 | Wasinowska et al. TCB | |
| | (PRAHEV (SEQ ID | 1: 73-75 (1997) | |
| | NO: 36)) | | |
| 2B-13 | Residues 38-43 | Wasinowska et al. TCB | |
| | (PRAHEV (SEQ ID | 1: 73-75 (1997) | |
| | NO: 36)) | | |
| 2B-11 | Residues 38-44 | Wasinowska et al. TCB | |
| | (PRAHEVS (SEQ ID | 1: 73-75 (1997) | |
| | NO: 37)) | | |
| NaM10-2H12 | Residues 38-45 | Rasamoelisolo et al. | |
| | (PRAHEVSE (SEQ ID | Vox Sang 72: 185-191 | |
| | NO: 38)) | (1997) | |
| NaM16-IB10 | Residues 38-45 | Rasamoelisolo et al. | |
| | (PRAHEVSE (SEQ ID | Vox Sang 72: 185-191 | |
| | NO: 38)) | (1997) | |
| NaM10-6G4 | Residues 38-45 | Rasamoelisolo et al. | |
| | (PRAHEVSE (SEQ ID | Vox Sang 72: 185-191 | |
| | NO: 38)) | (1997) | |
| A63-B/C2 | Within residues 46-55 | Karsten et al. | |
| | (ISVRTVYPPE (SEQ | International | |
| | ID NO: 39)) | Immunopharmacology | |
| | | 10: 1354-1360 (2010) | |
| 2B-21 | Residues 49-56 | Wasinowska et al. TCB | |
| | (RTVYPPEE (SEQ ID | 1: 73-75 (1997) | |
| | NO: 40)) | | |
| 2B-25 | Residues 49-56 | Wasinowska et al. TCB | |
| | (RTVYPPEE (SEQ ID | 1: 73-75 (1997) | |
| | NO: 40)) | | |
| 2B-9 | Residues 52-56 | Wasinowska et al. TCB | |
| | (YPPEE (SEQ ID NO: | 1: 73-75 (1997) | |
| | 41)) | | |
| 2B-20 | Residues 52-57 | Wasinowska et al. TCB | |
| | (YPPEEE (SEQ ID | 1: 73-75 (1997) | |
| | NO: 42)) | | |
| 2B-19 | Residues 52-58 | Wasinowska et al. TCB | |
| | (YPPEEET (SEQ ID | 1: 73-75 (1997) | |
| | NO: 43)) | | |
| NaM70-3C10 | Residues 53-57 | Lisowska Adv Exp Med | |
| | (PPEEE (SEQ ID NO: | Biology 491: 155-169 | |
| | 44)) | (2001) | |
| | | Rasamoelisolo et al. | |
| | | Hybridoma 17: 283-288 | |
| | | (1998) | |
| 2B-4 | Residues 53-58 | Wasinowska et al. TCB | |
| | (PPEEET (SEQ ID NO: | 1: 73-75 (1997) | |
| | 45)) | | |
| B14 (also known as | Between aa 56-67 | Ridgewell et al. | Available as Cat. No. |
| BRIC 14) | | Biochem J. 209: | 9413 from the |
| | | 273-276 (1983); | International Blood |
| | | Chasis et al. JBC | Group Reference Lab, |
| | | 107: 1351-1357 (1988) | Bristol UK |
| B89 (also known as | Includes residues 58-60 | Rasamoelisolo et al. | |
| BRIC 89) | | Vox Sang 72: 185-191 | |
| | | (1997) | |
| | | Gardner et al. | |
| | | Immunology 68: | |
| | | 283-289 (1989) | |
| R7 | Includes residues 58-60 | Rasamoelisolo et al. | |
| | | Vox Sang 72: 185-191 | |
| | | (1997) | |
| | | Anstee Eur. J. Immunol | |
| | | 12 (1982) | |
| BRIC 93 | Includes residues 58-60 | Gardner et al. | |
| | | Immunology 68: | |
| | | 283-289 (1989) | |

In some embodiments of any of the aspects, the polypeptide comprises an anti-GYPA antibody reagent comprising the CDRs of an antibody reagent selected from Table 3. In some embodiments of any of the aspects, the polypeptide comprises an anti-GYPA antibody reagent comprising the VL and VH sequences of an antibody reagent selected from Table 3. In some embodiments of any of the aspects, the polypeptide comprises an anti-GYPA antibody reagent selected from Table 3.

In some embodiments of any of the aspects, the polypeptide comprises an anti-GYPA antibody reagent comprising the CDRs of an antibody reagent selected from Table 3, R18, 1H4, and IH4v1. In some embodiments of any of the aspects, the polypeptide comprises an anti-GYPA antibody reagent comprising the VL and VH sequences of an antibody reagent selected from Table 3, R18, IH4, and IH4v1. In some embodiments of any of the aspects, the polypeptide comprises an anti-GYPA antibody reagent selected from Table 3, R18, 1H4, and IH4v1.

In some embodiments of any of the aspects, the polypeptide comprises an anti-GYPA antibody reagent comprising the CDRs of an antibody reagent selected from R18, IH4, IH4v1, 2B-21, 2B-25, 2B-9, 2B-20, 2B-19, NaM70-3C10, 2B-4, B14, B89, R7, and BRIC 93. In some embodiments of any of the aspects, the polypeptide comprises an anti-GYPA antibody reagent comprising the VL and VH sequences of an antibody reagent selected from R18, IH4, IH4v1, 2B-21, 2B-25, 2B-9, 2B-20, 2B-19, NaM70-3C10, 2B-4, B14, B89, R7, and BRIC 93. In some embodiments of any of the aspects, the polypeptide comprises an anti-GYPA antibody reagent selected from R18, IH4, IH4v1, 2B-21, 2B-25, 2B-9, 2B-20, 2B-19, NaM70-3C10, 2B-4, B14, B89, R7, and BRIC 93.

In some embodiments of any of the aspects, the polypeptide comprises an anti-GYPA antibody reagent comprising the CDRs of an antibody reagent selected from IH4, IH4v1, 2B-9, 2B-20, 2B-19, NaM70-3C10, 2B-4, B14, B89, R7, and BRIC 93. In some embodiments of any of the aspects, the polypeptide comprises an anti-GYPA antibody reagent comprising the VL and VH sequences of an antibody reagent selected from IH4, IH4v1, 2B-9, 2B-20, 2B-19, NaM70-3C10, 2B-4, B14, B89, R7, and BRIC 93. In some embodiments of any of the aspects, the polypeptide comprises an anti-GYPA antibody reagent selected from IH4, IH4v1, 2B-9, 2B-20, 2B-19, NaM70-3C10, 2B-4, B14, B89, R7, and BRIC 93.

In one aspect of any of the embodiments, described herein is a polypeptide comprising a) an anti-GYPA antibody reagent that binds the epitope of SEQ ID NO: 25 b) an erythropoietin, and c) a linker sequence separating the anti-GYPA antibody reagent and the erythropoietin. In some embodiments of any of the aspects, the anti-GYPA antibody reagent comprises one or more CDRs of IH4 (or IH4v1). In some embodiments of any of the aspects, the anti-GYPA antibody reagent comprises the three CDRs of IH4 (or IH4v1), e.g., SEQ ID NOs: 27-29. In some embodiments of any of the aspects, the anti-GYPA antibody reagent comprises a VHH or nanobody having the sequence of SEQ ID NO: 10 or 11.

In some embodiments of any of the aspects, the anti-GYPA antibody reagent comprises one or more CDRs of R18. In some embodiments of any of the aspects, the anti-GYPA antibody reagent comprises the six CDRs of R18. In some embodiments of any of the aspects, the anti-GYPA antibody reagent comprises the VL and/or VH sequence of R18. In some embodiments of any of the aspects, the anti-GYPA antibody reagent comprises the sequences of SEQ ID NOs: 49 and 50.

As used herein, “erythropoietin” or “EPO” refers to a cytokine produced primarily in the kidney and to a lesser extent the liver, and which stimulates erythropoiesis. EPO is a hormone that stimulates RBC production by binding to EPO-Rs on RBC precursors, and can cause a variety of other effects via EPO-Rs on other cell types, such as platelet activation and production, expression of tissue factor on endothelial cells, activation of the renin-angiotensin system, neuroprotection against hypoxia, and acceleration of tumor cell growth (Bunn Cold Spring Harb. Perspect. Med. (2013) 3). EPO plays multiple biological roles by binding to EPO receptors (EPO-R) on diverse cell types, including erythroid progenitors, macrophages, pro-megakaryocytes, cancer cells, and neurons (Jelkmann et al. Ann. Hematol. (2004) 83: 673-686; Bunn Cold Spring Harb. Perspect. Med. (2013) 3). Sequences for EPO nucleic acids and polypeptides are known for a number of species, including, e.g., human EPO (NCBI Gene ID: 2056) mRNA (e.g, NCBI Ref Seq: NM_00799.4) and polypeptides (e.g., NCBI Ref Seq: NP_00790.2 and SEQ ID NO: 17). A therapeutic goal of engineered proteins that include targeted EPO is to minimize the side effects of EPO by targeting the protein to red blood cell (“RBC”) precursors and away from other cell types. Recombinant EPO has been used for two decades to treat forms of anemia associated with endstage renal failure, AIDS, chemotherapy, or hemoglobinopathies (Jelkmann et al. Ann. Hematol. (2004) 83: 673-686; Bunn Cold Spring Harb. Perspect. Med. (2013) 3) and the compositions described herein can be used in place of recombinant EPO for any therapeutic use for which recombinant EPO is utilized.

In some embodiments of any of the aspects, the erythropoietin can be a human erythropoietin. In some embodiments of any of the aspects, the erythropoietin can be an erythropoietin with reduced binding affinity for its receptor relative to a wild-type erythropoietin, e.g., an erythropoietin with reduced binding affinity to its receptor relative to the wild-type human erythropoietin of SEQ ID NO: 16. Reduced binding affinity can comprise a binding affinity which is reduced 2× or more relative to the reference binding affinity, e.g., reduced 2×, 3×, 4×, 5×, 10×, 12×, 15×, 20× or more. In some embodiments of any of the aspects, reduced binding affinity is a reduction of 10× or more relative to the reference binding affinity of the erythropoietin of SEQ ID NO: 16. In some embodiments of any of the aspects, reduced binding affinity is a reduction of 12× or more relative to the reference binding affinity of the erythropoietin of SEQ ID NO: 16.

In some embodiments of any of the aspects, the erythropoietin comprises an erythropoietin having a sequence with at least one mutation at an amino acid residue of SEQ ID NO: 16 selected from R150, A30, H32, P87, W88, P90, R53, and E55. In some embodiments of any of the aspects, the erythropoietin comprises an erythropoietin having a sequence with at least 90% identity to SEQ ID NO: 16 and having at least one mutation corresponding to an amino acid residue of SEQ ID NO: 16 selected from R150, A30, H32, P87, W88, P90, R53, and E55. In some embodiments of any of the aspects, the erythropoietin comprises an erythropoietin having a sequence with at least 95% identity to SEQ ID NO: 16 and having at least one mutation corresponding to an amino acid residue of SEQ ID NO: 16 selected from R150, A30, H32, P87, W88, P90, R53, and E55. In some embodiments of any of the aspects, the erythropoietin comprises an erythropoietin having a sequence with at least 98% identity to SEQ ID NO: 16 and having at least one mutation corresponding to an amino acid residue of SEQ ID NO: 16 selected from R150, A30, H32, P87, W88, P90, R53, and E55. In some embodiments of any of the aspects, the erythropoietin comprises an erythropoietin having the sequence of SEQ ID NO: 16 except for one mutation corresponding to an amino acid residue of SEQ ID NO: 16 selected from R150, A30, H32, P87, W88, P90, R53, and E55.

In some embodiments of any of the aspects, the erythropoietin comprises an erythropoietin having a sequence with at least one mutation at an amino acid residue of SEQ ID NO: 16 wherein the at least one mutation is R150A, A30N, H32T, P87V, W88N, P90T, R53N, or E55T. In some embodiments of any of the aspects, the erythropoietin comprises an erythropoietin having a sequence with at least 90% identity to SEQ ID NO: 16 and having at least one mutation wherein the at least one mutation is R150A, A30N, H32T, P87V, W88N, P90T, R53N, or E55T. In some embodiments of any of the aspects, the erythropoietin comprises an erythropoietin having a sequence with at least 95% identity to SEQ ID NO: 16 and having at least one mutation corresponding to an amino acid residue of SEQ ID NO: 16 wherein the at least one mutation is R150A, A30N, H32T, P87V, W88N, P90T, R53N, or E55T. In some embodiments of any of the aspects, the erythropoietin comprises an erythropoietin having a sequence with at least 98% identity to SEQ ID NO: 16 and having at least one mutation corresponding to an amino acid residue of SEQ ID NO: 16 wherein the at least one mutation is R150A, A30N, H32T, P87V, W88N, P90T, R53N, or E55T. In some embodiments of any of the aspects, the erythropoietin comprises an erythropoietin having the sequence of SEQ ID NO: 16 except for one mutation corresponding to an amino acid residue of SEQ ID NO: 16 wherein the at least one mutation is R150A, A30N, H32T, P87V, W88N, P90T, R53N, or E55T.

In some embodiments of any of the aspects, the erythropoietin comprises the sequence of SEQ ID NO: 17. In some embodiments of any of the aspects, the erythropoietin is a murine or primate erythropoietin.

The term “linker” refers to any means, entity or moiety used to join two or more entities. A peptide linker is typically used to connect the EPO element and the antibody element.

The attachment of the anti-GYPA antibody reagent and the EPO (or activity element) can be by means of linkers, chemical modification, peptide linkers, chemical linkers, covalent or non-covalent bonds, or protein fusion or by any means known to one skilled in the art. The joining can be permanent or reversible. In some embodiments of any of the aspects, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention. According to the present invention, the polypeptide or fragments, derivatives or variants thereof, can be linked to the first fusion partner via any suitable means, as known in the art, see for example U.S. Pat. Nos. 4,625,014, 5,057,301 and 5,514,363, which are incorporated herein in their entirety by reference. For example, the polypeptide e can be covalently conjugated to the IgG1 Fc, either directly or through one or more linkers. In one embodiment, a polypeptide as disclosed herein is conjugated directly to the first fusion partner (e.g. Fc), and in an alternative embodiment, a polypeptide as disclosed herein can be conjugated to a first fusion partner (such as IgG1 Fc) via a linker, e.g. a transport enhancing linker.

A large variety of methods for conjugation of two polypeptides are known in the art. Such methods are e.g. described by Hermanson (1996, Bioconjugate Techniques, Academic Press), in U.S. Pat. Nos. 6,180,084 and 6,264,914 which are incorporated herein in their entirety by reference and include e.g. methods used to link haptens to carrier proteins as routinely used in applied immunology. Suitable methods for conjugation of two polypeptides include e.g. carbodimide conjugation (Bauminger and Wilchek, 1980, Meth. Enzymol. 70: 151-159) or as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate two polypeptides as disclosed herein, for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multi-step protocols can offer a great control of conjugate size and the molar ratio of components.

A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling.

Linkers of 5, 7, 17, 18, 29, 35 and 39 amino acids were tested and all found to functionally attach EPO to the various antibody elements. When an scFv is used, typically there is a linker of at least 15 amino acids between the VH and VL elements. It is preferable to use 18 or more amino acids between the VH and VL elements, as these longer linkers minimize the dimer formation. Linkers of lengths from 0 (no linker) to about 100 amino acids may be used. In some embodiments of any of the aspects, the linkers comprise or consist primarily of glycine and serine, but may include any amino acid. In some embodiments of any of the aspects, the linkers consist of glycine and serine.

The most typical configuration for an scFv-EPO fusion protein is VH-linker-VL-linker-EPO. However, other configurations such as EPO-linker-VH-linker-VL, EPO-linker-VL-linker-VH, and VH-linker-VL-linker-EPO are also possible. These are roughly similar in their spatial geometries, because movement and rotation around the linker between the scFv element allows the EPO element to assume a large number of conformations.

In addition, configurations in which the EPO element is in between the VH and VL elements may be constructed. These configurations are possible because the N- and C-termini of the EPO element are spatially close together, so that the VH and VL can easily pair. Specific configurations include VH-linker-EPO-linker-VL and VL-linker-EPO-linker-VH. Configurations in which EPO is in between the VH and VL cause the EPO element to be constrained in the orientations that it can adopt, which can be useful in situations where the dis-allowed conformations may lead to undesired binding events and side effects.

In some embodiments of any of the aspects, the linker sequence is no more than 17 amino acids in length, e.g., the linker is 1, 2, 3, 4, 5, 6, 7, 8, 9, 10, 11, 12, 13, 14, 15, 16, or 17 amino acids in length. In some embodiments of any of the aspects, the linker sequence is at least 5 amino acids in length. In some embodiments of any of the aspects, the linker sequence is 5-35 amino acids in length. In some embodiments of any of the aspects, the linker sequence is 5-7 amino acids in length, e.g., the linker is 5, 6, or 7 amino acids in length. In some embodiments of any of the aspects, the linker sequence is at 7 or fewer amino acids in length, e.g., the linker is 1, 2, 3, 4, 5, 6, or 7 amino acids in length. Exemplary but non-limiting linker sequences are provided herein, e.g., at SEQ ID NOs. 12-15, 20, 22, and 24.

In one aspect of any of the embodiments, described herein is a polypeptide comprising an anti-GYPA antibody reagent, a linker of no more than 14 nm in length, and an activity element. In one aspect of any of the embodiments, described herein is a polypeptide comprising an anti-GYPA antibody reagent, a linker sequence of no more than 17 amino acids, and an activity element.

An activity element may be a receptor binding protein (or functional portion or domain thereof) that binds to one or more naturally-occurring receptors on a cell surface, thereby mediating signaling to the cell (e.g., via signal transduction). In some embodiments, the activity element is a portion of a cytokine or a hormone that is sufficient to bind to a receptor on the surface of the target cell and induce an activity in the cell. In some embodiments, the cytokine or hormone is a four-helix-bundle protein. In some embodiments, the activity element is a variant form (e.g., it is mutated) that has an intrinsic binding to its receptor that is weak as compared to the wild-type protein, to the point that binding of the chimeric protein to a cell is driven by the binding of the anti-GYPA antibody reagent to its receptor. In some embodiments, the activity element is a variant of a naturally occurring protein that activates cells by binding to one or more cell surface receptors. The variant is selected such that it has reduced or no cell activating properties in the absence of the anti-GYPA antibody reagent. In some embodiments, cell activation results in stimulation of red blood cell production.

As used herein, a “mutation” refers to a change in the nucleotide sequence encoding the activity element, relative to a wild-type form of the gene, and includes substitution, deletion, and insertion mutations. A change in the nucleotide sequence may or may not lead to a change in the amino acid sequence, the three-dimensional structure of the protein, and/or the activity of the protein, relative to the wild-type form of the protein. In some embodiments a mutation may be a naturally occurring variant of the gene. In some embodiments, the activity element comprises a mutation of at least 1, 2, 3, 4, 5, 6, 7, 8, 9, 10, or more amino acids relative to a wild type/non-mutated activity element. In some embodiments a mutation may be a single amino acid substitution, two or more amino acid substitutions, one or more deletions, one or more insertions, or any combination of two or more thereof, in the protein sequence of the activity element. It will be understood that the selection of a suitable mutation in an activity element for the creation of a chimeric molecule will depend on multiple factors and in some embodiments will need to be determined empirically for different proteins.

It should be appreciated that variant activity elements of the invention may have a reduced binding affinity for their receptor(s) without a loss (or without a significant loss) of signal function (e.g., they substantially or completely retain their ability to promote signal transduction when bound to their receptor(s) even though their affinity for the receptor(s) may be significantly reduced). It also should be appreciated that the reduced binding affinity of the variant activity element preferably does not result in a protein element that will not bind to its natural receptor(s), for example, due to steric hindrance or charge repulsion or other negative interaction between the variant activity element and its natural receptor(s), even when the anti-GYPA antibody reagent binds to a target molecule on the same cell (thereby increasing the local concentration of the variant activity element in the vicinity of its natural receptor(s)).

In some embodiments, appropriate levels of reduced binding affinity can be obtained by introducing one or more mutations in charged or hydrophilic amino acids (or amino acids thought to be pointing outward) that have the effect of shortening the side chain of the amino acid(s). According to aspects of the invention, the charged or hydrophilic side chains are likely to be pointing outward and not into the middle of the protein. Reducing the size of the amino acid side chain(s) removes a contact, but does not create steric hindrance that would completely block binding or signaling. In some embodiments, mutants may be created in an activity element, and those that reduce but do not abolish binding may be selected (e.g., using one or more binding and/or activity assays known to one of skill in the art) and used to construct a chimeric activator. In some embodiments, one or more mutation in an activity element may reduce binding of the activity element to one monomer of a receptor and not affect the binding of the activity element to one or more other monomers of the receptor.

A variant activity element may include one or more naturally occurring and/or engineered mutations that result in reduced binding to one or more (e.g., all) natural receptors that are bound by the wild-type activity element. For example, the binding affinity of the variant activity element for one or more of its receptors may be at least 2-fold lower and preferably at least 5-fold lower or 8-fold lower (e.g., at least 10-fold lower, about 10-50-fold lower, about 50-100-fold lower, about 100-150-fold lower, about 150-200-fold lower, or more than 200-fold lower) than the binding affinity of the wild-type activity element for its natural receptor(s). As a result, in some embodiments the activity element by itself (e.g., not part of a fusion or chimeric protein comprising an anti-GYPA antibody reagent) is significantly less active (e.g., substantially inactive) because it cannot bind or has reduced binding to its receptor(s). In some embodiments, the variant activity element stimulates less signaling to the cell as compared to a wild type activity element (e.g., an activity element that does not have the variation/mutation). In some embodiments, the variant activity element induces at least 2-fold, at least 3-fold, at least 4-fold, at least 5-fold, at least 10-fold, at least 20-fold, at least 50-fold, at least 100-fold or less signaling to the cell as compared to a wild type activity element. Accordingly, the polypeptides, e.g., fusion polypeptides of the invention are useful to avoid unwanted side effects caused by the activity element binding to its natural receptor on non-target cells. However, the activity element is active on target cells because the anti-GYPA antibody reagent provides the missing binding affinity required for activation.

As used herein, “binding affinity” refers to the apparent association constant or KA. The KA is the reciprocal of the dissociation constant (KD). In some embodiments, the targeting element or the activity element described herein may have a binding affinity (KD) of at least 10-5, 10-6, 10-7, 10-8, 10-9, 10-10 M, 10-11 M or lower for the corresponding receptor or targeting molecule.

In some embodiments of any of the aspects, the activity element is erythropoietin.

In addition, proteins of the invention may include one or more additional peptide elements (at the N-terminus, the C-terminus, or within the protein) for purification and/or detection (e.g., peptide tags), stability, function (e.g., secretion and or other function), etc., or any combination thereof.

In one aspect of any of the embodiments, described herein is a nucleic acid encoding a polypeptide described herein, e.g., a polypeptide comprising an anti-GYPA antibody reagent, an activity element or erythropoietin, and optionally a linker. In one aspect of any of the embodiments, described herein is a vector comprising a nucleic acid encoding a polypeptide described herein, e.g., a polypeptide comprising an anti-GYPA antibody reagent, an activity element or erythropoietin, and optionally a linker. A nucleic acid encoding a polypeptide can further comprise expression control elements, e.g., promoters, enhancers and the like operably linked to the sequence encoding the polypeptide.

In one aspect of any of the embodiments, described herein is a cell comprising 1) a nucleic acid encoding a polypeptide described herein, e.g., a polypeptide comprising an anti-GYPA antibody reagent, an activity element or erythropoietin, and optionally a linker or 2) a vector comprising said nucleic acid. The cell can be a eukaryotic or prokaryotic cell, e.g., for expression of the polypeptide in a bacterial, yeast, or human cell. Suitable cells/cell lines for protein expression are well known in the art.

Proteins of the invention are often produced by expression of a DNA construct in a mammalian cell. Other cell types such as bacteria, yeast, and insect cells may also be used. An important consideration in production of the EPO fusion proteins of the invention is that the level of sialic acid modification on the N-linked oligosaccharides should be as high as possible. To maximize sialic acid modification, cell lines such as CHO (Chinese Hamster Ovary) cells or BHK (Baby Hamster Kidney) cells can be used. These cell lines are available from the American Type Culture Collection.

To express a protein of the invention, a DNA sequence encoding the protein sequence of interest is operably linked to elements that promote protein expression and secretion, such as enhancer(s), core promoter elements such as the TATA element, a Kozak sequence for optimal ribosome binding and translation, 3′ end elements such as a polyA addition sequence and transcription terminator, introns (optional), and a signal sequence to promote translocation into the endoplasmic reticulum and eventual secretion from the cell.

For purposes of DNA propagation and cell line construction, the DNA encoding the Targeted EPO protein is generally embedded in a plasmid that also includes a bacterial selectable marker such as ampicillin resistance, a bacterial origin of replication, and a selectable marker that can be used in mammalian cells, such as dihydrofolate reductase, hygromycin resistance, neomycin resistance, or zeocin resistance. SEQ ID NO: 30 is a typical expression cassette for a Targeted EPO protein of the invention.

SEQ ID NO: 30
CGATGTACGGGCCAGATATACGCGTTGACATTGATTATTGACTAGTTATTA
ATAGTAATCAATTACGGGGTCATTAGTTCATAGCCCATATATGGAGTTCCG
CGTTACATAACTTACGGTAAATGGCCCGCCTGGCTGACCGCCCAACGACCC
CCGCCCATTGACGTCAATAATGACGTATGTTCCCATAGTAACGCCAATAGG
GACTTTCCATTGACGTCAATGGGTGGACTATTTACGGTAAACTGCCCACTT
GGCAGTACATCAAGTGTATCATATGCCAAGTACGCCCCCTATTGACGTCAA
TGACGGTAAATGGCCCGCCTGGCATTATGCCCAGTACATGACCTTATGGGA
CTTTCCTACTTGGCAGTACATCTACGTATTAGTCATCGCTATTACCATGGT
GATGCGGTTTTGGCAGTACATCAATGGGCGTGGATAGCGGTTTGACTCACG
GGGATTTCCAAGTCTCCACCCCATTGACGTCAATGGGAGTTTGTTTTGGCA
CCAAAATCAACGGGACTTTCCAAAATGTCGTAACAACTCCGCCCCATTGAC
GCAAATGGGCGGTAGGCGTGTACGGTGGGAGGTCTATATAAGCAGAGCTCT
CTGGCTAACTAGAGAACCCACTGCTTACTGGCTTATCGAAATTAATACGAC
TCACTATAGGGAGACCCAAGCTGGCTAGCCACCATGGAGACAGACACACTC
CTGCTATGGGTACTGCTGCTCTGGGTTCCAGGTTCCACTGGTCAGGTCCAA
CTGCAGGAGAGCGGCGGGGGGTCAGTTCAGGCGGGGGGGAGTCTGCGGTTG
AGCTGCGTAGCTTCAGGCTACACTGACAGCACCTACTGCGTGGGATGGTTT
CGGCAGGCACCCGGCAAGGAACGAGAGGGCGTTGCACGGATCAACACTATC
TCCGGTCGGCCTTGGTACGCAGATAGTGTTAAGGGACGGTTTACTATTAGT
CAGGATAACTCTAAGAATACCGTCTACCTTCAGATGAATAGCCTGAAACCG
GAAGACACGGCTATTTACTATTGCACCCTTACAACTGCCAACAGCAGAGGG
TTTTGTTCTGGGGGATATAACTACAAAGGACAGGGGACCCAAGTCACTGTC
AGCTCTGGTGGTGGTTCCGCTCCACCTAGATTGATTTGTGATTCCAGAGTT
TTGGAAAGATACTTGTTGGAAGCTAAGGAGGCTGAAAATATTACTACTGGT
TGTGCTGAACATTGTTCTTTGAACGAGAATATTACTGTTCCAGATACTAAG
GTTAACTTTTACGCTTGGAAGAGAATGGAAGTTGGTCAGCAAGCTGTTGAA
GTTTGGCAAGGTTTGGCTTTGTTGTCTGAAGCTGTTTTGAGAGGTCAAGCT
TTGTTGGTTAATTCTTCTCAACCATGGGAACCATTGCAATTGCATGTTGAT
AAGGCTGTTTCTGGTTTGAGATCTTTGACTACCTTGTTGAGAGCTTTGGGT
GCTCAAAAGGAAGCTATTTCTCCTCCAGATGCTGCTTCTGCCGCTCCATTG
AGAACTATTACTGCTGATACTTTTAGAAAGTTGTTTAGAGTTTACTCTAAC
TTCTTGGCCGGTAAGTTGAAGTTGTACACTGGTGAAGCTTGTAGAACTGGT
GATCGGGGGCCCGAACAAAAACTCATCTCAGAAGAGGATCTGAATAGCGCC
GTCGACCATCATCATCATCATCATTGAGTTTAAACCCGCTGATCAGCCTCG
ACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCT
GGGGA

In this sequence, the distinct segments consist of a CMV promoter element; a T7 promoter element; a Kozak sequence (gcc(G/A)ccATGG) and upstream sequences compatible with efficient translation; a murine IgK leader sequence to promote secretion from the cell; a sequence encoding a mature fusion protein consisting of the IH4v1 nanobody and a 5 amino acid linker and EPO R150A; a sequence encoding a cMyc tag, a 6×His tag and a stop codon; and a BGH polyadenylation sequence.

To produce protein for initial testing of candidate forms of Targeted EPO, mammalian cells such as HEK293F (Human Embryo Kidney) cells are transiently transfected with an expression plasmid comprising a sequence such as SEQ ID: 30 Example 11 demonstrates a typical transient transfection. The resulting protein can be tested for expression level, aggregation and in vitro activity in cell-based assays. However, Targeted EPO proteins expressed in HEK293F cells generally have reduced levels of sialic acid compared to natural EPO and to the desired product that should be used in vivo. One effect of less of sialic acid is to reduce the negative charge on the protein. Because the binding of EPO to EPO receptor is partly driven by the relative positive charges on EPO and negative charges on the receptor, reducing the number of negatively charged sialic acid residues, and thus increasing the binding of the EPO element for its receptor relative to and EPO protein that has been produced from CHO or BHK cells. This increased receptor binding has the in vivo effect of increasing EPO receptor-mediated endocytosis and degradation, thus decreasing the plasma half-life of EPO-based molecules and paradoxically decreasing the in vivo activity.

Proteins of the invention may also be produced from CHO cells by transient or stable transfection. The amount of material produced from CHO cells upon transient transfection is generally less than from a HEK293 cell transfection, but the sialic acid content is higher.

A useful type of CHO cells is the CHO-DG44 line, which is DHFR deficient. According to the invention, a plasmid encoding a Targeted EPO and also encoding a DHFR gene is inserted into the CHO-DG44 cell line by selection for growth in the absence of hypoxanthine and thymidine. The expression of a gene of interest can be further improved by growth in the presence of methotrexate. Yeo et al. (Biotechnology Journal 12:1700175 (2017)) describes detailed procedures for such cell line construction and is incorporated by reference herein.

Targeted EPO proteins of the invention are purified by standard procedures. Example 11 illustrates specific methods. For example, the Targeted EPOs are purified by nickel-IDA or cobalt affinity methods (see, e.g., “His60 Ni Superflow Resin & Gravity Columns User Manual” ClonTech Laboratories, Inc. PT5017-1 (031716) followed by size exclusion chromatography using endotoxin-free reagents. Goldwasser et al. (Proc. Nat. Acad. Sci. USA Vol. 68, No. 4, pp. 697-698, April 1971) also describes purification methods for erythropoietin itself; these may be readily adapted for purification of Targeted EPO. Each of the foregoing references is incorporated by reference herein in its entirety.

Merely by way of non-limiting example, to formulate the Targeted EPO proteins of the invention, the following buffers and excipients may be used: phosphate-buffered saline, human serum albumin, arginine, mannitol, sorbitol, citrate, Tween such as Tween80, and so on. Formulation of protein drugs is well-known in the art of pharmaceutical development.

The activity and pharmacological efficacy of the polypeptides described herein (e.g., Targeted EPO proteins) can be measured by any of the assays described herein. By way of example, purified Targeted EPO, crude cell supernatants, and material from intermediate stages in purification may be tested by methods including, but not limited to, liquid-chromatography/mass-spectrometry, dynamic light scattering, analytical size exclusion chromatography, SDS-PAGE, isoelectric focusing, lectin binding, sandwich ELISA in which one moiety is captured and the other is detected, binding to EPO receptor, and binding to glycophorin A. These methods are used to determine purity and extent of contamination with host cell protein, extent of aggregation, chemical degradation of amino acid side chains, proteolysis, extent of glycosylation and particularly the level of sialic modification, and correct folding of, e.g., the EPO moiety and of the IH4 antibody element. These methods are generally well known in the state of the art for pharmaceutical industry protein characterization. Glycophorin A binding as assayed with red blood cells, is described in Example 6.

Assays for contamination by microbial material include testing for endotoxin by standard Limulus amebocyte lysate (LAL) methods (e.g., those available from Charles River (Wilmington, Mass.), e.g., on the world wide web at criver.com/products-services/qc-microbial-solutions/endotoxin-testing/lal-reagents-accessories, and culturing for live microbes by standard microbiological techniques.

The erythropoietic activity of an EPO element in a fusion protein can be assayed using cells that express the EPO receptor. The cells may optionally also express glycophorin A. For example, the cell lines TF-1 and UT-7 express both EPO receptor and glycophorin A. Taylor et al. (PEDS 1-10 (2010)) and Burrill et al. (PNAS 113:5245-5250 (2016)) describe the use of these cell lines to assay various forms of Targeted EPO, and are incorporated by reference herein. Other cell lines such as MCF-7 express EPO receptor but not glycophorin A. These cell lines may be used to specifically measure EPO activity independently of membrane-localizing effects of the antibody element and glycophorin A.

Typically, such cell-based assays are performed as follows. Cells are distributed into microtiter plates such as a 96-well plate. They are allowed to adhere and begin to grow. Well before the cells become confluent, the medium is changed to a somewhat poor medium, and various concentrations of Targeted EPO protein are added to the wells. The extent of cell growth is measured about 3 days later by standard methods.

Red blood cells may be used to assay glycophorin A binding. Typically, a Targeted EPO protein is added to a preparation of red blood cells; a labeled secondary antibody that recognizes either a peptide tag, the EPO element, or the targeting element of the Targeted EPO is added for detection; cells are washed; and then the cells are analyzed by flow cytometry. Use of a tertiary labeled antibody is sometimes necessary. Example 6 illustrates details of such methods. Assays based on binding to red blood cells provide several types of information. For example, batch-to-batch variation may be tested and bad batches of drug product can be identified. Additionally, during a research phase, binding to red blood cells can be used to measure antibody accessibility of drug, which correlates with the potential of the EPO element of a bound drug to contact other cells. FIG. 5A illustrates this principle.

The polypeptides described herein can also be tested in vivo, e.g., using animal models to demonstrate activity and safety. Typically, an animal is injected intravenously, and the bleeding time and/or levels of reticulocytes and reticulated platelets can be measured, e.g., as described in Example 7.

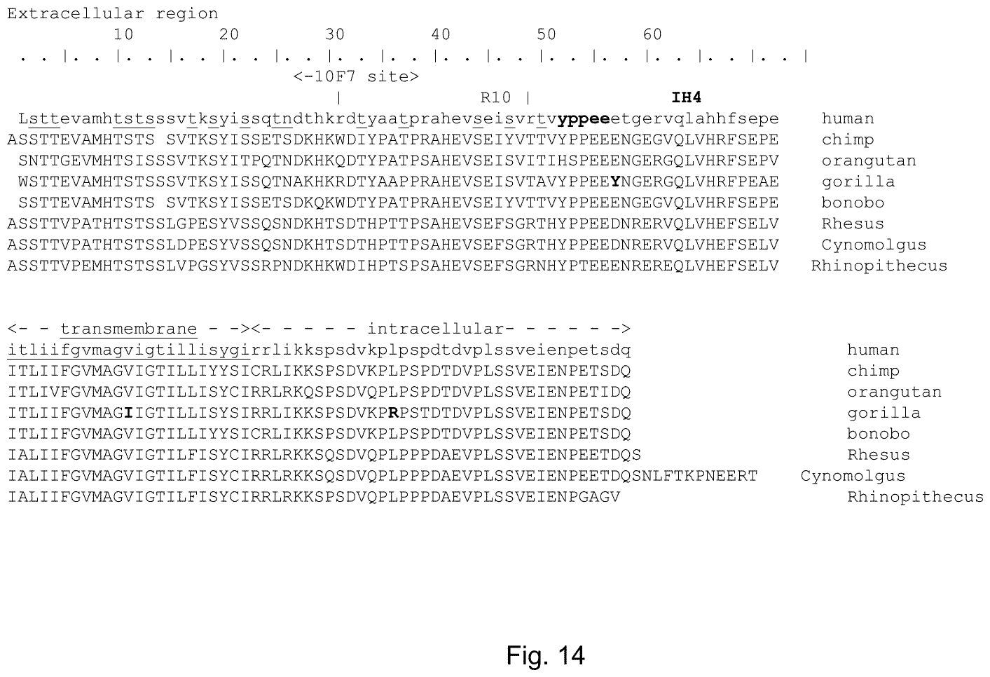

It is important to consider the species specificity of binding for both the EPO element and the glycophorin-binding element when choosing an animal test system. Among mammals, human EPO generally acts on the EPO receptors of other animals, since this system is highly conserved. In contrast, the extracellular portion of glycophorin A is not highly conserved. For example, the mouse and human glycophorin A extracellular domains are not recognizably related, while the transmembrane and intracellular domains can easily be aligned. Some animals do not encode a functional glycophorin A. Even within primates, the sequences of glycophorin A show significant variation. The alignment depicted in FIG. 14 provides a comparison of the glycophorin A sequences of humans, chimpanzees, orangutans, gorillas, bonobo chimpanzees, rhesus monkeys, cynomolgus monkeys (aka the crab-eating macaque), and the black snub-nosed monkey (Rhinopithecus bieti). Also indicated are O-linked and N-linked glycosylation sites (all in the extracellular domain, underlined), the epitopes for the anti-glycophorin antibodies R10, 10F7, and IH4, and the transmembrane and intracellular domains. Inspection of the glycophorin sequences indicates that the epitopes for these various antibodies (all of which were selected for binding to human glycophorin A) are altered in some non-human primates, and thus that these primates would not be appropriate for testing forms of Targeted EPO that use the corresponding antibody. For example, the gorilla has the mutations Asp27Lys, Thr28Lys, and Thr37Pro in the region of the 10F7 epitope, and thus gorillas glycophorin may not be bound by glycophorin A based on this analysis. Similarly, the IH4 epitope appears to be Tyr52, Pro53, Pro54, Glu55, Glu56 (Habib et al.), which is altered in the orangutan, and Rhinopithecus; binding to the gorilla sequence could also be disrupted due to the Glu57Tyr change that might lead to steric hindrance. Thus, orangutan, Rhinopithecus and gorilla would not be the first choice for primate test systems, while chimps, rhesus monkeys and cynomolgus monkeys would likely be better primate test systems because the glycophorin A on their red blood cells should be bound by IH4.

It is also useful to perform animal testing in a rodent model system. The glycophorin A sequences from non-primate mammals is generally so divergent from the human sequence that antibodies to human glycophorin A are not expected to bind at all. Auffray et al. (Blood 97:2972-2979 (2001)) developed a transgenic mouse that expresses human glycophorin A. The breeding of this mouse strain is described by Auffray et al and by Burrill et al., which are incorporated by reference herein in their entireties. In brief, the human glycophorin A transgene causes no deleterious phenotype in the mice when heterozygous, but is homozygous lethal. During breeding, the presence of the transgene may be monitored by PCR testing, or by staining of blood samples with a labeled antibody directed against human glycophorin A, followed by flow cytometry. From a cross of a transgene/+ male with a wild-type female, about half of the offspring will carry the transgene.

The testing of forms of Targeted EPO to demonstrate stimulation of red blood cell production and lack of platelet production is described by Burrill et al., and in Example 6 and 7.

Long-term toxicity testing may also be performed in transgenic mice or in primates. For example, glycophorin A-transgenic mice are injected with a Targeted EPO such as IH4-(5aa)-EPO(R150A) twice per week for eight weeks. Blood is withdrawn periodically and the plasma fraction is tested for the presence of anti-drug antibodies. In addition, the hematocrit, platelet, reticulocyte and reticulated platelet counts are determined. Antibodies to the drug may develop. Such antibodies may be, for example, in the form of weak, non-neutralizing antibodies that do not inhibit the function of the drug, and in fact have the effect of extending the plasma half-life of the drug. Alternatively, drug-specific neutralizing antibodies that block drug activity may form. These can be detected by ELISA in the plasma of the treated mice. Such antibodies will have the in vivo effect of preventing drug action, so that red blood cell levels and reticulocyte levels will not be elevated during long-term treatment. The worst-case scenario is the formation of anti-drug antibodies that cross-react with the endogenous EPO of the animal. In such a case, the animal may become anemic and in some cases may become aplastic (complete failure to produce red blood cells), which is generally fatal if untreated.

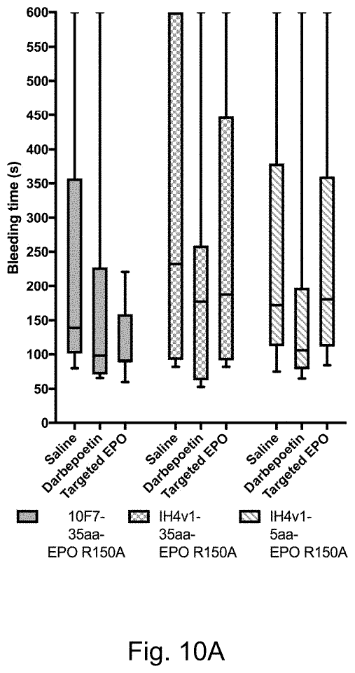

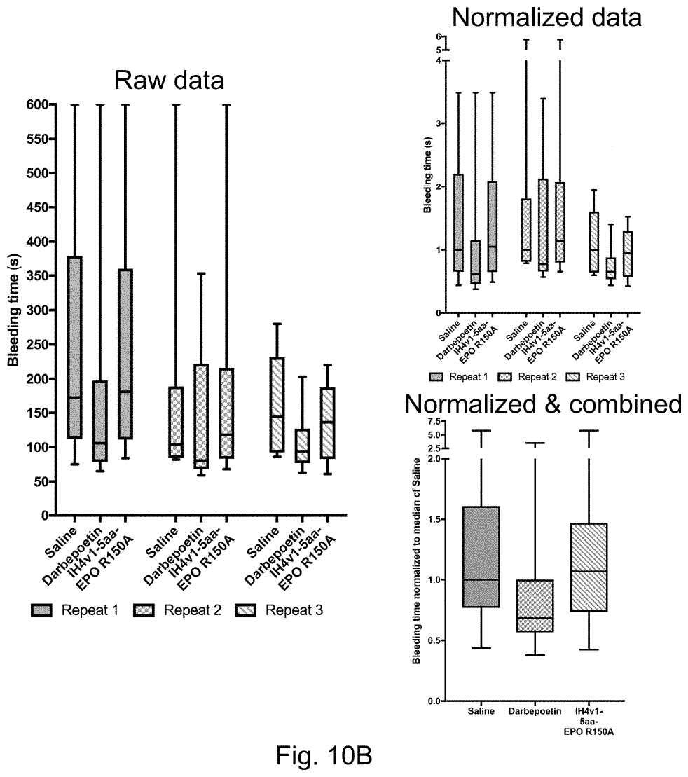

Another set of toxicological animal tests relate to blood clotting. Erythropoietin itself enhances blood clotting and markers relating to blood clotting. In human clinical trials there is evidence that patients treated with erythropoiesis-stimulating agents (ESAs) such as epoetin alpha, epoetin beta, or darbepoetin show an increased frequency of stroke, cardiac arrest, and deep vein thrombosis. These observations are the motivation for the present invention. In addition, Kirkeby et al. (Thromb Haemost 99:720-728 (2008))) showed that erythropoietin treatment of rats had the following effects: (1) shortening the bleeding time as measured in a tail transection assay; (2) increasing plasma P-selectin; (3) increasing platelet sensitivity to thrombin receptor agonist peptides; (4) increasing mean platelet volume; (5) increasing levels of P2Y₁, P2Y₁₂, MEK1/2, and GSK3beta; and other effects. As described herein, treatment of glycophorin A-transgenic mice with erythropoietin or darbepoetin shortens the bleeding time by about 30% (FIGS. 10A, 10B). A further finding described herein is that different forms of Targeted EPO have dramatically different effects in the tail transection/bleeding time assay. These results are summarized below.

TABLE 2

| Treatment | Mouse genotype | Shortening bleeding time? |
|---|---|---|
| Saline | Wild-type | No (baseline-defining treatment) |
| | (non-transgenic) | |
| Saline | Wild-type | No (baseline-defining treatment) |
| | (transgenic) | |
| Erythropoietin | Wild-type | Yes |
| | (non-transgenic) | |
| Darbepoetin | Wild-type | Yes |
| | (non-transgenic) | |
| Erythropoietin | Wild-type | Yes |
| | (transgenic) | |
| Darbepoetin | Wild-type | Yes |
| | (transgenic) | |
| 10F7-(35AA)- | Wild-type | Yes |
| EPO(R150A) | (transgenic) | |
| 10F7-(35AA)- | Wild-type | No |
| EPO(R150A) | (non-transgenic) | |
| IH4v1-(35AA)- | Wild-type | Yes |
| EPO(R150A) | (transgenic) | |
| IH4v1-(5AA)- | Wild-type | No |
| EPO(R150A) | (transgenic) | |

The tail transection assay of bleeding time can be performed on mice as follows. On day 0, mice are injected with a test protein (or vehicle). When testing an unknown protein, it is important to also include mice that are injected with saline or PBS vehicle as a negative control, and EPO or darbepoetin as a positive control. Typically, 10 mice per dose group are used. It is important that the experiment is performed in a blind manner. For example, one experimenter performs the injections of proteins into the mice, maintains the key, performs the injection of anaesthetic, and then hands the mice in a random order to a second, blinded experimenter. The second experimenter performs the tail transfections and measures the bleeding time. On day 1, the tail transection and bleeding time is measured. In principle, the tail transection could be performed on day 2, 3, 4 or later. However, the advantage of performing the measurement 1 day after treatment is that after only 24 hours, the level of circulating red blood cells will not have changed, so effects on blood clotting are due to direct effects on some element of the clotting system, and not due to changes in blood viscosity.

Tail transection can be performed as follows. The mice are first anaesthetized using anaestheticsketamine and xylazine. These anaesthetics are chosen because they are thought to not affect blood clotting. Acepromazine is not used because it has the effect of reducing clotting. Mice are weighed, and mice are then injected with 120-160 mg ketamine/kg and 10-16 mg xylazine/kg of body weight. For older and heavier mice, sometimes an additional injection of about 25% of the first injection was required. After a mouse became unresponsive to a stimulus such as significant pressure to a hind foot, the mouse is placed on a heated pad on a platform over a waterbath. The waterbath is maintained at 37° C. 50-ml blue-cap tubes (Sarstedt) are filled with 50 mls of a solution of 0.85 to 0.9% NaCl that has been equilibrated to 37° C. in a separate waterbath. The animal is placed on a Chux pad or equivalent.

A position on the tail that is 3 millimeters from the tip, not counting hair, is marked with a felt-tip pen using calipers. (The tail is also inspected for signs of bruising that may be due to fighting, and data from such a mouse is discarded if the transected tail does not bleed at all. The decision to discard the data must be made in a blinded manner). The tail is transected with a flat razor blade using a section of the blade that has not been used previously. Within 1 second, the transected tail is placed in a tube with pre-warmed saline, and then the body of the mouse is placed on the heated pad above the water bath. At the moment that the tail is transected by the blinded experimenter, the non-blinded experimenter starts a timer. The body of the mouse is then positioned on the heated pad so that only the tip of the tail—about 0.5 to 2 mm—is in the saline and the rest is in the air. When observing the bleeding tail, the tube should be rotated so that the white stripe is behind the tail, providing contrast, and the room should be well-lit. The rack holding the 50-ml tube should be white or yellow to provide contrast. The bleeding time is recorded by noting when bleeding stops, and then observing the submerged tail for up to one minute. If bleeding re-starts within this minute, the first recorded time is not counted. Bleeding may stop and re-start several times. If the tail is still bleeding when 10 minutes have elapsed, the time is recorded as 10 minutes.

The median and mean bleeding times are calculated for each treatment group. Calculating the median has the advantage that extreme events, such as 10 minute timepoints, do not disproportionately contribute to the calculation.

As described in the Examples herein, mice dosed with targeted EPO exhibited elevated RBC levels, with only minimal platelet effects. This in vivo selectivity depended on the identity of the GYPA epitope being targeted and the linker sequence length. In a therapeutic context, the compositions and methods described herein permit higher restorative doses of EPO without platelet-mediated side effects, and also can improve drug pharmacokinetics. These results demonstrate how rational drug design can improve in vivo specificity, with potential application to diverse protein therapeutics.

In one aspect of any of the embodiments, described herein is a method of increasing erythropoiesis comprising contacting a red blood cell with a polypeptide described herein, e.g., a Targeted EPO protein. The red blood cell can be in vitro or in vivo. In one aspect of any of the embodiments, the methods described herein relate to increasing erythropoiesis in a subject in need thereof by administering to the subject a polypeptide described herein, e.g., a Targeted EPO protein.

In one aspect of any of the embodiments, the methods described herein relate to treating a subject having or diagnosed as having anemia with a polypeptide described herein, e.g., a Targeted EPO protein. Subjects having anemia can be identified by a physician using current methods of diagnosing anemia. Symptoms and/or complications of anemia which characterize these conditions and aid in diagnosis are well known in the art and include but are not limited to, fatigue, weakness, pale or yellowish skin, irregular heartbeat, shortness of breath, dizziness, chest pain, cold extremities, and headache. Tests that may aid in a diagnosis of, e.g. anemia include, but are not limited to, a blood count and phenotypic analysis of red blood cells. A family history of anemia, or exposure to risk factors for anemia can also aid in determining if a subject is likely to have anemia or in making a diagnosis of anemia. In some embodiments of any of the aspects, a subject with anemia or in need of erythropoiesis can be a subject having or diagnosed as having chronic renal failure or altitude sickness or who has received chemotherapy.

The compositions and methods described herein can be administered to a subject having or diagnosed as having, e.g., anemia. In some embodiments, the methods described herein comprise administering an effective amount of compositions described herein, e.g. a polypeptide as described herein to a subject in order to alleviate a symptom of anemia. As used herein, “alleviating a symptom” is ameliorating any condition or symptom associated with the disease or condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of the therapeutic composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay described herein. The dosage can be determined by a physician and adjusted, as necessary, to suit observed effects of the treatment.

In some embodiments, the technology described herein relates to a pharmaceutical composition comprising a polypeptide as described herein, e.g., a Targeted EPO protein, and optionally a pharmaceutically acceptable carrier. In some embodiments, the active ingredients of the pharmaceutical composition comprise a polypeptide as described herein, e.g., a Targeted EPO protein as described herein. In some embodiments, the active ingredients of the pharmaceutical composition consist essentially of a polypeptide as described herein, e.g., a Targeted EPO protein as described herein. In some embodiments, the active ingredients of the pharmaceutical composition consist of a polypeptide as described herein, e.g., a Targeted EPO protein as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a polypeptide as described herein, e.g., a Targeted EPO protein as described herein.

In some embodiments, the pharmaceutical composition comprising a polypeptide as described herein, e.g., a Targeted EPO protein as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a polypeptide as described herein, e.g. a Targeted EPO protein as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, a polypeptide as described herein, e.g. a Targeted EPO protein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, a polypeptide as described herein, e.g., a Targeted EPO protein described herein is administered as a monotherapy, e.g., another treatment for anemia or to increase erythropoiesis is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include iron supplements, transfusions, and RBC transfusions.

In certain embodiments, an effective dose of a composition comprising a polypeptide as described herein, e.g., a Targeted EPO protein as described herein can be administered to a patient once. In certain embodiments, an effective dose of a composition comprising a polypeptide as described herein, e.g., a Targeted EPO protein can be administered to a patient repeatedly. For systemic administration, subjects can be administered a therapeutic amount of a composition comprising a polypeptide as described herein, e.g., a Targeted EPO protein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a polypeptide as described herein, e.g., a Targeted EPO protein can be administered over a period of time, such as over a 5 minute, 10 minute, 15 minute, 20 minute, or 25 minute period.

The dosage ranges for the administration of a polypeptide as described herein, e.g., a Targeted EPO protein, according to the methods described herein depend upon, for example, the form of the polypeptide, its potency, and the extent to which symptoms, markers, or indicators of a condition described herein are desired to be reduced, for example the percentage reduction desired for anemia symptoms or the extent to which, for example, red blood cell numbers are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a polypeptide as described herein, e.g. a Targeted EPO protein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in in vitro or animal models of a condition described herein, e.g., according to the various assays described in detail elsewhere herein.

By way of non-limiting example, a human patient may be treated with a protein of the invention as follows. A patient with chronic kidney disease who is being treated by dialysis may be given a Targeted EPO of the invention by iv administration, post-dialysis, using the same iv line that has been established for the dialysis. For an adult human patient, a typical initial dose of a Targeted EPO protein is about 400 micrograms, administered once per two weeks. For patients that respond with a rapid rise in hemoglobin (such as more than 1 g/dL in any 2-week period), the dose of Targeted EPO should be reduced by 25% or more as needed to reduce rapid responses. For patients who do not respond adequately—for example if the hemoglobin has not increased by more than 1 g/dL after 4 weeks of therapy—the dose should be increased by 25%. The response to Targeted EPO varies from patient to patient and hemoglobin levels should be monitored.

In some embodiments of any of the aspects, the starting doses of a polypeptide as described herein, e.g. a Targeted EPO protein is about 10 micrograms to 10 milligrams, 50 micrograms to 3.2 milligrams, 100 micrograms to 1.6 milligrams, 200 micrograms to 800 micrograms, or about 400 micrograms. The Targeted EPO of the invention is typically administered by iv infusion, or subcutaneous, intramuscular or intradermal injections, either once per week, once per two weeks, or once per month. These are typical doses for adult patients. Depending on the weight of the patient in kilograms, the invention provides starting doses of about 0.2 micrograms/kg to 200 micrograms/kg, 0.75 micrograms/kg to 50 micrograms/kg, 1.5 micrograms/kg to 24 micrograms/kg, 3 micrograms/kg to 12 micrograms/kg, or about 6 micrograms/kg.

As Targeted EPO proteins are erythropoiesis-stimulating agents (ESAs), general considerations about when to treat, methods of administration and so on are the same as for other ESAs such as Epogen™ or Procrit™, and are described in the package inserts that accompany these drugs. The package insert information may also be obtained, for example, on the world wide web at pi.amgen.com/˜/media/amgen/repositorysites/pi-amgen-com/epogen/epogen_pi_hcp_english.pdf. In addition, for self-injecting pre-dialysis patients administering a Targeted EPO protein at home, the methods for safe self-injection of Epogen available on the world wide web at pi.amgen.com/˜/media/amgen/repositorysites/pi-amgen-com/epogen/epogen_piu_pt_english.pdf also apply. It should be noted that the black-box warnings in package inserts for other ESAs such as Epogen, Procrit, and Aranesp™ do not apply to the Targeted EPO molecules of the invention, particularly those using the IH4 V domain, a linker of 7 amino acids or less, and a mutation in the EPO element corresponding to Arg150Ala.

By way of non-limiting example, a 70-kg adult human patient with chronic kidney disease and uncontrolled hypertension can be treated as follows. The patient has a hematocrit of less than 25, corresponding to a hemoglobin level of less than about 8 gm/dL, before treatment is initiated. The patient is given between 10 micrograms and 10 milligrams, but preferably about 400 micrograms, of a Targeted EPO protein as described herein. The protein is administered after a dialysis procedure, using the same iv line that was set up for dialysis; thus, the administration is intravenous. After 2 weeks, the patient's hemoglobin level is measured and found to be 8.5 gm/dL. This is considered to be an acceptable rate of increase, so at this time the patient is given another dose of the same protein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of anemia or chronic kidney disease. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Other boundaries defining CDRs overlapping with the Kabat CDRs have been described by Padlan (FASEB J. 9:133-139 (1995)) and MacCallum (J Mol Biol 262(5):732-45 (1996)) and Chothia (J. Mol. Biol. 196:901-917 (1987) and Nature 342:877-883 (1989)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat defined CDRs. The CDR's identified herein, e.g., SEQ ID NOs 1-6 are identified by the Kabat system (see, e.g. FIGS. 4 and 5).

The term “antigen-binding portion” of an antibody refers to one or more portions of an antibody as described herein, said portions) still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a dAb portion consisting of a VH domain or of VH, CH1, CH2, DH3, or VH, CH2, CH3 (dAbs, or single domain antibodies, comprising only V_{L} domains have also been shown to specifically bind to target eptiopes). Although the two domains of the Fv portion, namely VL and VH, are encoded by separate genes, they may further be linked to one another using a synthetic linker, e.g., a poly-G4S amino acid sequence (‘G4S’ disclosed as SEQ ID NO: 51), and recombinant methods, making it possible to prepare them as a single protein chain in which the VL and VH regions combine in order to form monovalent molecules (known as single chain Fv (ScFv)). The term “antigen-binding portion” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody, antibody reagent, or antigen-binding portion thereof as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cystein residue, a marker peptide and a C-terminal polyhistidinyl, e.g., hexahistidinyl tag (‘hexahistidinyl tag’) in order to produce bivalent and biotinylated scFv molecules.

In some embodiments, the antibody, antibody reagent, or antigen-binding portion thereof described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin). The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody, or antigen-binding portion thereof as described herein. Such functional activities include binding to cancer cells and/or anti-cancer activity. Additionally, a polypeptide having functional activity means the polypeptide exhibits activity similar, but not necessarily identical to, an activity of a reference antibody, antibody reagent, or antigen-binding portion thereof as described herein, including mature forms, as measured in a particular assay, such as, for example, a biological assay, with or without dose dependency. In the case where dose dependency does exist, it need not be identical to that of the reference antibody, antibody reagent, or antigen-binding portion thereof but rather substantially similar to the dose-dependence in a given activity as compared to the reference antibody, antibody reagent, or antigen-binding portion thereof as described herein (i.e., the candidate polypeptide will exhibit greater activity, or not more than about 25-fold less, about 10-fold less, or about 3-fold less activity relative to the antibodies, antibody reagents, and/or antigen-binding portions described herein).

In some embodiments, the antibody reagents (e.g., antibodies) described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

In some embodiments, the antibody, antibody reagent, and/or antigen-binding portion thereof is an isolated polypeptide. In some embodiments, the antibody, antibody reagent, and/or antigen-binding portion thereof is a purified polypeptide. In some embodiments, the antibody, antibody reagent, and/or antigen-binding portion thereof is an engineered polypeptide.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

“Glycophorin A” or “GYPA” (sometimes referred to as GPA or CD235a in the art) is a sialoglycoportein found on erythrocyte membranes which carries antigenic determinants for the MN and Ss blood groups. Sequences for GYPA in a number of species are known in the art, e.g., human GYPA (NCBI Gene ID: 2993) mRNA (NM 001308187.1; NM_001308190.1; NM_002099.8) and protein (NP_001295116.1; NP_001295119.1; NP_002090.4) sequences.

In some embodiments, a nucleic acid encoding a polypeptide as described herein (e.g. a Targeted EPO polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optomized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. anemia. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A polypeptide comprising a) an anti-GYPA antibody reagent that binds the epitope of SEQ ID NO: 25, b) an erythropoietin, and c) a linker sequence separating the anti-GYPA antibody reagent and the erythropoietin.
2. The polypeptide of paragraph 1 wherein the anti-GYPA antibody reagent comprises one or more CDRs of IH4.
3. The polypeptide of any of paragraphs 1-2, wherein the anti-GYPA antibody reagent comprises the three CDRs of IH4.
4. The polypeptide of any of paragraphs 1-3, wherein the anti-GYPA antibody reagent comprises a VHH having the sequence of SEQ ID NO: 10 or 11.
5. The polypeptide of paragraph 1, wherein the anti-GYPA antibody reagent comprises one or more CDRs of an antibody reagent selected from R18, IH4, IH4v1, and Table 3.
6. The polypeptide of paragraph 1, wherein the anti-GYPA antibody reagent comprises the CDRs of an antibody reagent selected from R18, IH4, IH4v1, and Table 3.
7. The polypeptide of paragraph 1, wherein the anti-GYPA antibody reagent comprises the VH and VL sequences of an antibody reagent selected from R18, IH4, IH4v1, and Table 3.
8. The polypeptide of paragraph 1, wherein the anti-GYPA antibody reagent comprises an antibody reagent selected from R18, IH4, IH4v1, and Table 3.
9. The polypeptide of any of paragraphs 5-8, wherein the antibody reagent is selected from R18, IH4, IH4v1, 2B-21, 2B-25, 2B-9, 2B-20, 2B-19, NaM70-3C10, 2B-4, B14, B89, R7, and BRIC 93.
10. The polypeptide of any of paragraphs 5-8, wherein the antibody reagent is selected from IH4, IH4v1, 2B-9, 2B-20, 2B-19, NaM70-3C10, 2B-4, B14, B89, R7, and BRIC 93.
11. The polypeptide of any of paragraphs 1-10, wherein the erythropoietin comprises at least one mutation at an amino acid residue of SEQ ID NO: 16 selected from R150, A30, H32, P87, W88, P90, R53, and E55.
12. The polypeptide of paragraph 11, wherein the at least one mutation is R150A, A30N, H32T, P87V, W88N, P90T, R53N, or E55T.
13. The polypeptide of any of paragraphs 1-12, wherein the linker sequence is no more than 17 amino acids in length.
14. The polypeptide of any of paragraphs 1-13, wherein the linker sequence is at least 5 amino acids in length.
15. The polypeptide of any of paragraphs 1-13, wherein the linker sequence is 5-35 amino acids in length.
16. The polypeptide of any of paragraphs 1-13, wherein the linker sequence is 5-7 amino acids in length.
17. The polypeptide of any of paragraphs 1-13, wherein the linker sequence is 7 or fewer amino acids in length.
18. A polypeptide comprising an anti-GYPA antibody reagent, a linker sequence of no more than 17 amino acids, and an activity element.
19. The polypeptide of paragraph 18, wherein the anti-GYPA antibody comprises IH4 or 10F7.
20. The polypeptide of any of paragraphs 18-19, wherein the anti-GYPA antibody comprises one or more CDRs of IH4.
21. The polypeptide of any of paragraphs 18-20, wherein the anti-GYPA antibody reagent comprises the three CDRs of IH4.
22. The polypeptide of any of paragraphs 18-21, wherein the anti-GYPA antibody reagent comprises a VHH having the sequence of SEQ ID NO: 10 or 11.
23. The polypeptide of paragraph 18, wherein the anti-GYPA antibody reagent comprises one or more CDRs of an antibody reagent selected from R18, IH4, IH4v1, and Table 3.
24. The polypeptide of paragraph 18, wherein the anti-GYPA antibody reagent comprises the CDRs of an antibody reagent selected from R18, IH4, IH4v1, and Table 3.
25. The polypeptide of paragraph 18, wherein the anti-GYPA antibody reagent comprises the VH and VL sequences of an antibody reagent selected from R18, IH4, IH4v1, and Table 3.
26. The polypeptide of paragraph 18, wherein the anti-GYPA antibody reagent comprises an antibody reagent selected from R18, IH4, IH4v1, and Table 3.
27. The polypeptide of any of paragraphs 23-26, wherein the antibody reagent is selected from R18, IH4, IH4v1, 2B-21, 2B-25, 2B-9, 2B-20, 2B-19, NaM70-3C10, 2B-4, B14, B89, R7, and BRIC 93.
28. The polypeptide of any of paragraphs 23-26, wherein the antibody reagent is selected from IH4, IH4v1, 2B-9, 2B-20, 2B-19, NaM70-3C10, 2B-4, B14, B89, R7, and BRIC 93.
29. The polypeptide of any of paragraphs 18-28, wherein the activity element is erythropoietin.
30. The polypeptide of any of paragraphs 18-29, wherein the erythropoietin comprises at least one mutation at an amino acid residue of SEQ ID NO: 16 selected from R150, A30, H32, P87, W88, P90, R53, and E55.
31. The polypeptide of paragraph 30, wherein the at least one mutation is R150A, A30N, H32T, P87V, W88N, P90T, R53N, or E55T.
32. The polypeptide of any of paragraphs 18-31, wherein the linker sequence is no more than 7 amino acids in length.
33. The polypeptide of any of paragraphs 18-31, wherein the linker sequence is at least 5 amino acids in length.
34. The polypeptide of any of paragraphs 18-31, wherein the linker sequence is 5-7 amino acids in length.
35. A method of increasing erythropoiesis comprising contacting a red blood cell with a polypeptide of any of paragraphs 1-34.
36. A method of treating anemia in a subject in need thereof, the method comprising administering a polypeptide of any of paragraphs 1-34 to the subject.
37. A polypeptide of any of paragraphs 1-34 for use in treating anemia in a subject in need thereof.
38. The method or polypeptide of any of paragraphs 35-37, wherein the subject has or is diagnosed as having chronic renal failure or altitude sickness or has received chemotherapy.
39. A nucleic acid encoding the polypeptide of any of paragraphs 1-34.
40. A vector comprising the nucleic acid of paragraph 39.
41. A cell comprising the nucleic acid of paragraph 39 or the vector of paragraph 40.

### EXAMPLES

### Example 1: Design, Expression, and Purification of Exemplary Targeted EPO Molecules

Targeted EPO for stimulation of erythropoiesis without thrombosis, inflammation, or cancer progression which are present in the current wild-type EPO products was constructed. An activity element (mutated EPO) and a targeting element (an anti-GYPA antibody, IH4v1) were fused via a flexible glycine-serine linker.

The mutated EPO used in this Example has a Arg150Ala (R150A) mutation which weakens the receptor binding affinity and erythropoietic activity by 12-16-fold compared to the wild-type EPO in vitro.

IH4v1 is a camelid single-domain antibody against human GYPA and has an additional amino acid (Thr118) in the framework region 4 compared to the original IH4 described in the U.S. Pat. No. 9,879,090. IH4(v1) recognizes ₅₂YPPEE₅₆ in the extracellular domain of human GYPA, which is highly expressed in the RBC late precursors (CFU-E and later stages) and mature RBCs. Its binding to this specific epitope prevents pro-inflammatory signaling in RBCs, while other GYPA antibodies, such as 10F7MN and R10, that bind at around ₃₄YAATP₃s, cause inflammatory reactions, such as stiffening of RBC membrane, phosphorylation of Band3, and production of ROS and ATP.

The minimum linker length was chosen based on the following considerations. The published structure of the EPO-EPOR complex, a structural model of the extracellular domain of human GYPA bound by IH4(v1), and the published epitope of IH4(v1) indicated that the EPO-binding site on EPOR is about 50 Angstroms from the cell surface, and the GYPA-bound IH4(v1) is about 40-50 Angstroms from the cell surface, assuming that IH4(v1) binds at the reasonable angle of 30-60 degrees from the cell surface. Thus it was contemplated that a linker of about 10 Angstroms or longer could allow the binding of GYPA-tethered EPO to EPOR on RBC precursors in cis.

Without wishing to be bound by theory, the advantages of a short vs. long linker, as described herein can be based on the following considerations. The IH4(v1) binding epitope is about 22.5 Angstroms from the cell surface and the estimated length of IH4(v1) and EPO is about 100 Angstroms. The number of GYPA molecules on the surface of a mature RBC and the average surface area of a mature RBC indicated that the average distance between the two adjacent GYPA molecules is only about 140 Angstroms. The height of the extracellular domain of GYPA is predicted to be about 100-130 Angstroms from the cell surface, based on its protein sequence and a structural model, and the N-terminus of GYPA is highly glycosylated, so that a shorter linker would have an advantage of placing GYPA-tethered EPO below the highly packed and negatively charged N-termini of GYPA and sterically blocking the in trans interaction between GYPA-tethered EPO on a mature RBC and EPOR on a nearby non-target cell.

Based on these biological, structural and quantitative considerations, five targeted EPO molecules were constructed:
1. IH4v1—(5-aa Gly-Ser linker)—EPO R150A
2. IH4v1—(7-aa Gly-Ser linker)—EPO R150A
3. IH4v1—(17-aa Gly-Ser linker)—EPO R150A
4. IH4v1—(35-aa Gly-Ser linker)—EPO R150A
5. 10F7MN—(35-aa Gly-Ser linker)—EPO R150A

These proteins were expressed in HEK293F (Invitrogen) and CHO-S (Invitrogen) cells for in vitro and in vivo experiments, respectively. HEK293F and CHO-S cells were transfected with a plasmid composed of a mammalian-expressing vector containing the secretion tag (pSecTag2A, Invitrogen), targeted EPO and epitope tags (6×His and c-Myc) using the transfection reagents, 293fectin™ (Gibco™) and FreeStyle™ MAX (Gibco™), respectively.

Dihydrofolate reductase (DHFR)-deficient CHO DG44 cells (Invitrogen) were used to generate stable production cell lines. CHO DG44 cells were transfected with a plasmid composed of a mammalian-expressing vector containing the secretion tag and the DHFR gene (pOptiVEC™ Invitrogen), targeted EPO and epitope tags (6×His and c-Myc) using the transfection reagent, FreeStyle MAX™.

Targeted EPO fusion proteins were expressed and secreted into cell culture medium. They were purified by nickel or cobalt resins using the His60 Ni or HisTalon™ kit, respectively (Takara). The starting cell supernatant and each purification fraction were analyzed by SDS-PAGE followed by Coomassie Blue staining. The purification eluents containing the fusion proteins were combined, desalted into endotoxin-free PBS (Teknova: 137 mM NaCl, 1.4 mM KH₂PO₄, 4.3 mM Na₂HPO₄, and 2.7 mM KCl, pH 7.4) using Econo-Pac™ 10DG columns (Bio-Rad), and concentrated to <1 mL using Macrosep Advance™ centrifugal device (Pall Corp.).

For in vivo experiments, proteins expressed by CHO-S or CHO DG44 cells purified by the described process and contaminating proteins were further removed by size-exclusion chromatography (SEC) on Superdex™ 200 10/300 GL columns (GE Healthcare) using an AKTA™ FPLC system (GE Healthcare) and endotoxin-free PBS as the running buffer. Desired protein fractions were combined and concentrated to <1 mL using Macrosep Advance™ centrifugal device.

Proteins produced by CHO-S or CHO DG44 cells contained legumain, an asparaginyl endopeptidase, even after SEC-FPLC. About 30-40% and 5% of the total protein purified by the nickel and cobalt resins, respectively, was legumain.

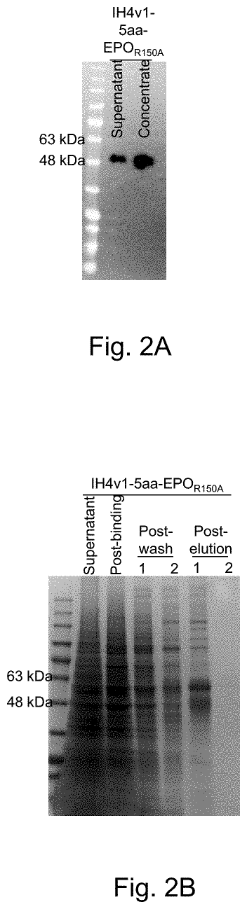

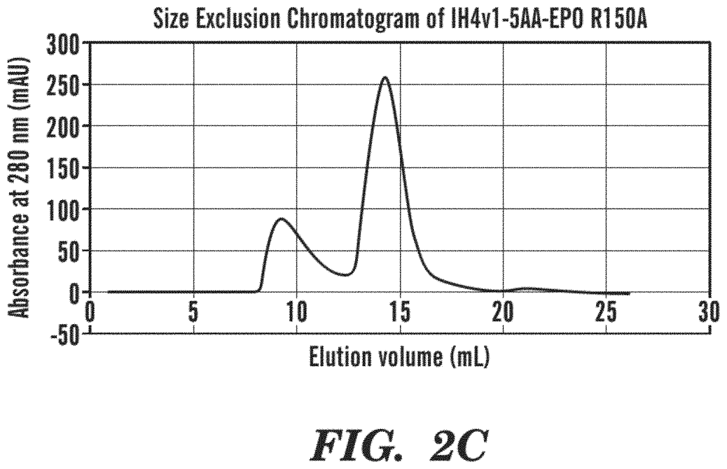

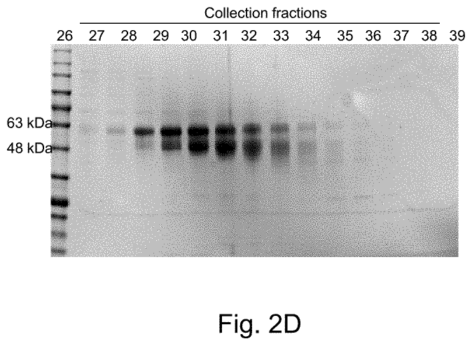

Protein concentration and purity (>90% for HEK293F-produced, >60% for CHO-produced proteins) were verified by SDS-PAGE followed by Coomassie Blue staining (FIG. 2D). N-linked glycosylation was verified by PNGase F (New England Biolabs) digestion followed by SDS-PAGE and Coomassie Blue staining. Sialylation of EPO produced by CHO cells was verified by isoelectric focusing followed by Western blotting. Proteins were stored at 4° C. throughout the described process. They were ultimately flash-frozen in liquid nitrogen and stored at −80° C. as aliquots.

### Example 2: Biological Activity and Potency of the Targeted EPO Fusion Proteins In Vitro

Targeted EPO fusion proteins were tested for their ability to stimulate the proliferation of TF-1 cells, which express GYPA and EPOR. 9.0×10³ TF-1 cells per well were seeded in a 96-well plate in 90 uL per well of RPMI-1640 with serum and antibiotics (no GM-CSF). The purified targeted EPO fusion proteins were serially diluted by 10-fold (10⁻¹⁴ to 10⁻⁷ M) and added to the cells. After 72 hours of incubation in the 37° C., 5% CO₂ incubator, 20 uL per well of MTS reagent (Promega) was added to cells. After 4 hours in the 37° C., 5% CO₂ incubator, cell proliferation was measured by reading absorbance at 490 nm on a BioTek Synergy™ Neo HTS microplate reader.

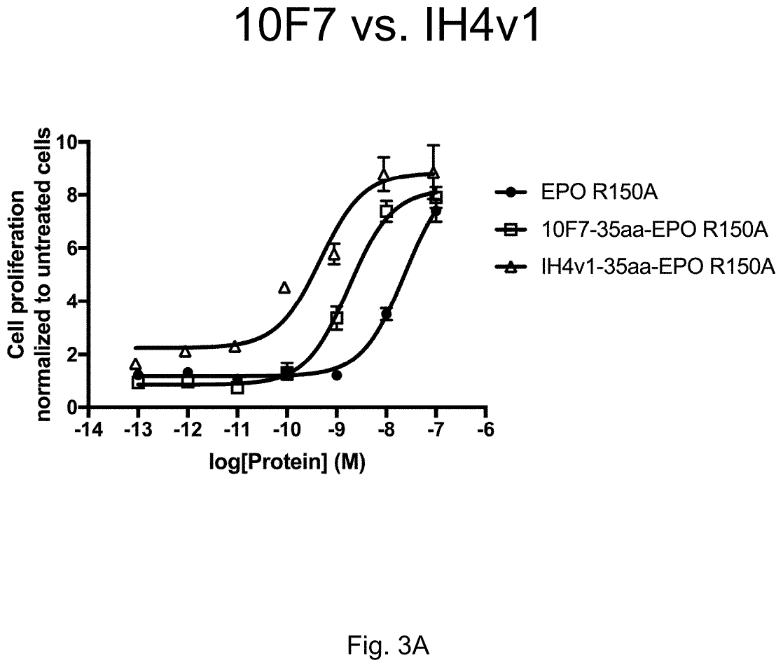

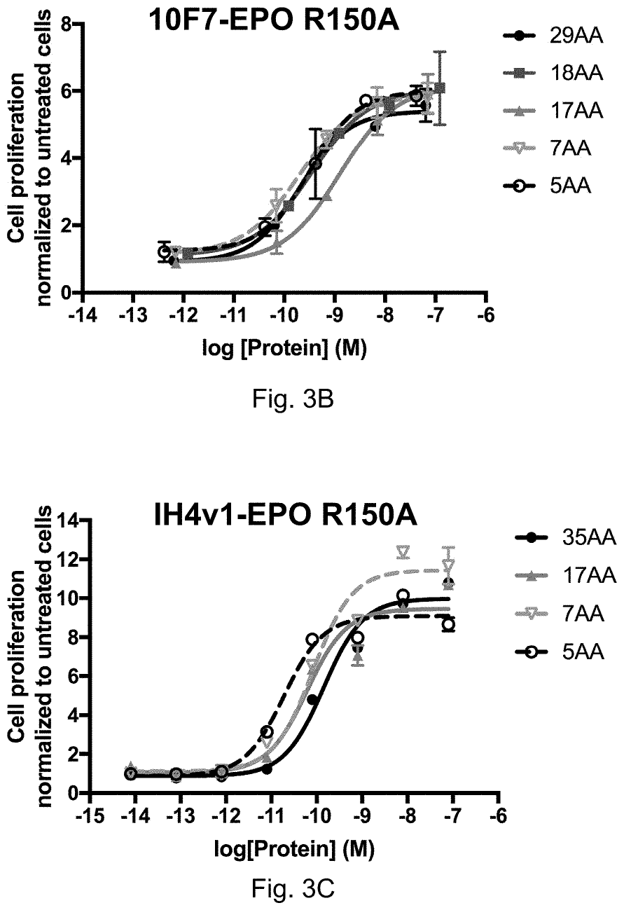

Results shown in FIG. 3A indicate that a new targeted EPO fusion protein with the new anti-GYPA antibody, IH4v1, rescued the activity of mutated EPO (R150A) and is about 8-fold more potent than the previous targeted EPO fusion protein containing the pro-inflammatory anti-GYPA antibody, 10F7MN, in stimulating TF-1 cell proliferation. IH4v1-EPO R150A fusion proteins with different linker lengths had about the same potency and efficacy in TF-1 proliferation, as shown in FIG. 3C. The new targeted EPO fusion proteins in general were about 5-fold more potent than the wild-type EPO product, Epogen™ (Amgen).

### Example 3: Effect of Anti-GYPA Antibody on RBC

Binding of some anti-GYPA antibodies such as 10F7MN and R10 causes increased rigidity of RBC membrane, phosphorylation of band 3, and production and release of ROS and ATP. Based on a model of extracellular domain of human GYPA structure, the published structure of band 3, and the published binding epitopes of GYPA antibodies, a new anti-GYPA antibody, IH4(v1), was hypothesized not to cause the described pro-inflammatory effects in RBCs and chosen as the new targeting element. The level of phosphorylated band 3 in mature RBCs was measured as an indicator of such pro-inflammatory reactions by the binding of an anti-GYPA antibody. Band 3 is an anion exchanger protein that reside in the RBC membrane. It serves a critical function of exchanging Cl⁻ and HCO₃⁻ in and out of the RBC during the gas exchange. It is physically associated with RBC cytoskeletal proteins, such as protein 4.1R, protein 4.2 and ankyrin, and affects membrane stability by changing the way it associates with the neighboring membrane proteins and cytoskeletal proteins.

Different anti-GYPA antibodies were tested for their effect on phosphorylation of band 3 in mature RBCs. Human blood was obtained by a finger prick. RBCs were isolated from whole blood by centrifugation at 500×g for 5 min at RT and washed twice with PBS-glucose buffer (endotoxin-free PBS, 5 mM glucose, pH 7.4). Then anti-GYPA antibodies, 10F7MN and IH4v1, were added to the packed RBC and incubated at 37° C. for 15 min. The concentration of each antibody was determined by its K_{D}, such that about 80% of the total GYPA on a RBC would be bound. Then RBCs were lysed and RBC proteins were denatured and reduced using Novex Tris-Glycine SDS sample buffer (Invitrogen) and NuPAGE sample reducing reagent (Invitrogen). The lysates were heated at 100° C. for 2 min and analyzed by SDS-PAGE followed by Western blotting. The proteins were transferred to a nitrocellulose membrane. The membrane was blocked using 5% BSA for 1 hr at 4° C. and probed with the HRP-conjugated anti-phospho-Tyrosine antibody (p-Tyr-1000, Cell Signaling Technology).

RBCs treated with 10F7MN exhibited a higher level of phosphorylated band 3 than those treated with buffer or IH4v1. 10F7MN shares the binding epitope with R10, which had already been shown to increase phosphorylation of band 3. On the other hand, IH4v1 has a different binding epitope, which is about 20-amino-acid closer to the membrane than that of 10F7MN or R10. This suggests that the binding of anti-GYPA antibody signals and induces phosphorylation of band 3 in an epitope-dependent manner. Furthermore, this indicates that IH4v1 is preferred over 10F7MN as a targeting element of the targeted EPO because the binding of 10F7MN to GYPA has higher chances of destabilizing RBC membrane and leading to pro-inflammatory reactions in the blood vessels.

### Example 4: Linker Length-Dependent Accessibility of RBC-Bound EPO

The length of the flexible glycine-serine linker determines the geometric space in which the cell surface-bound EPO explores. A long linker increases accessibility of EPO by the neighboring cells and allows for receptor binding in trans, whereas a short linker reduces with accessibility of EPO and limits receptor binding to occur in cis.

Targeted EPO fusion proteins of varying linker lengths were assayed for their ability to present RBC-bound EPO to the outside molecules. Human blood was obtained by a finger prick. RBCs were isolated from whole blood by centrifugation at 500×g for 5 min at RT and washed twice with PBS-glucose buffer (endotoxin-free PBS, 5 mM glucose, pH 7.4). Then targeted EPO fusion proteins are added to the packed RBCs and kept on ice for 1 hr to allow for binding. The concentration of each fusion protein was determined by K_{D} of IH4v1 for GYPA, such that about 80% of the total GYPA on a RBC would be bound. Mature RBCs express GYPA but not EPOR, so when targeted EPO fusion proteins are added, they are bound to the cell surface via targeting element and GYPA. After the 1 hr incubation, RBCs were washed twice with FACS buffer (R&D Systems) and were incubated with PE-conjugated anti-6×His tag antibody (Abcam) for 30 min on ice. Then RBCs were washed twice with FACS buffer and added to a 96-well U-bottom plate for flow cytometric analysis. The 6×His tag is at the C-terminus of EPO, which is located closed to the junction between EPO and the linker in the three-dimensional structure, and therefore, is a good indicator of how accessible EPO is to the neighboring cells or molecules. The fluorescence was measured on a LSRFortessa SORP flow cytometer equipped with an optional HTS sampler (BD Biosciences) using the following filter configuration: PE excitation, 561/50 mW; emission filter, BP 582/15.

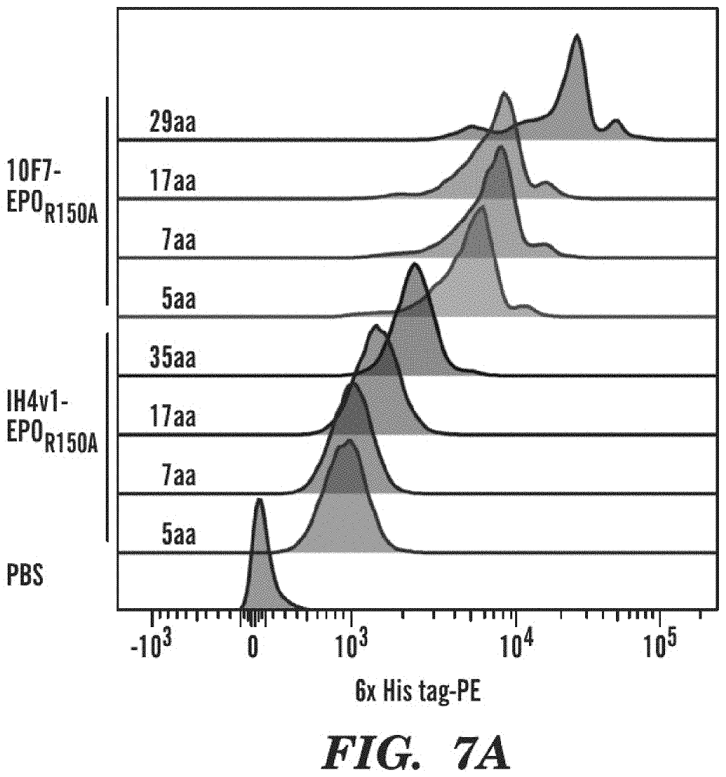

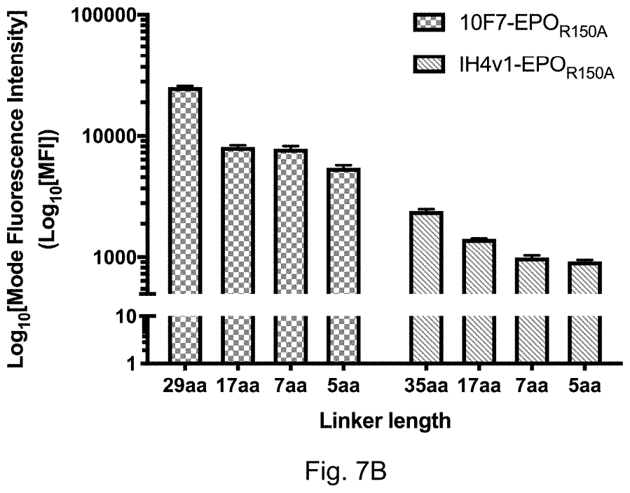

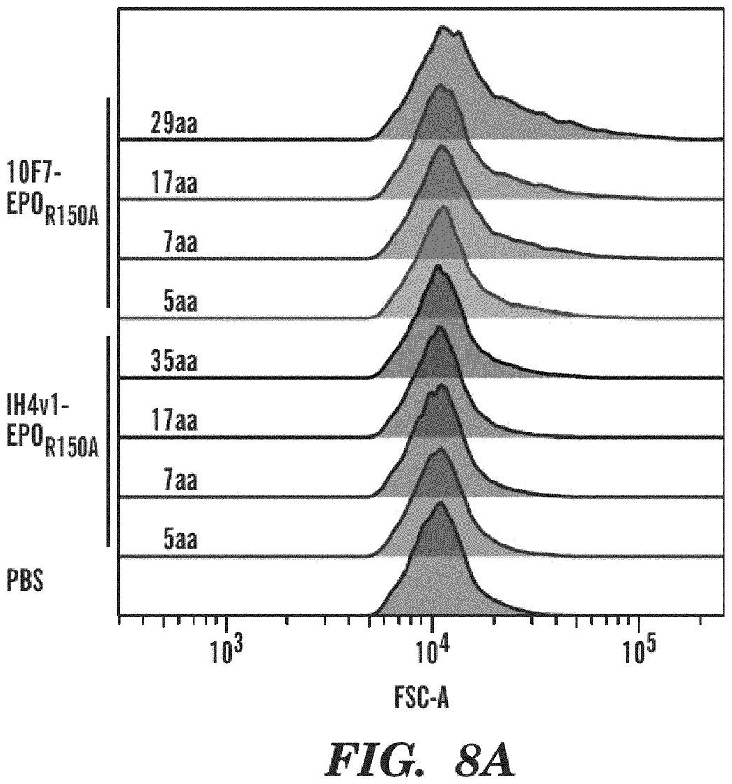

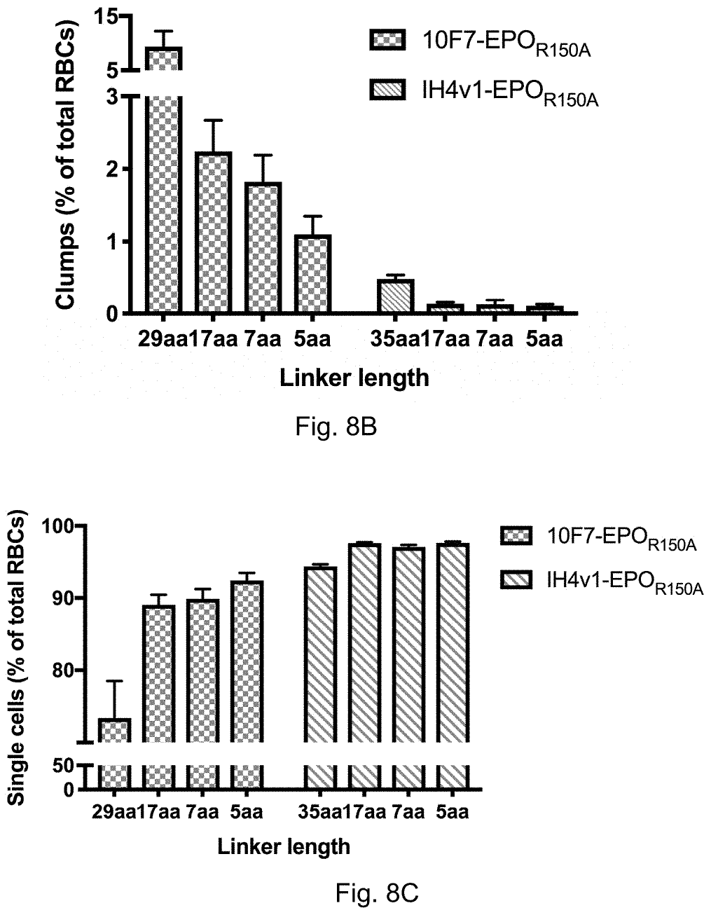

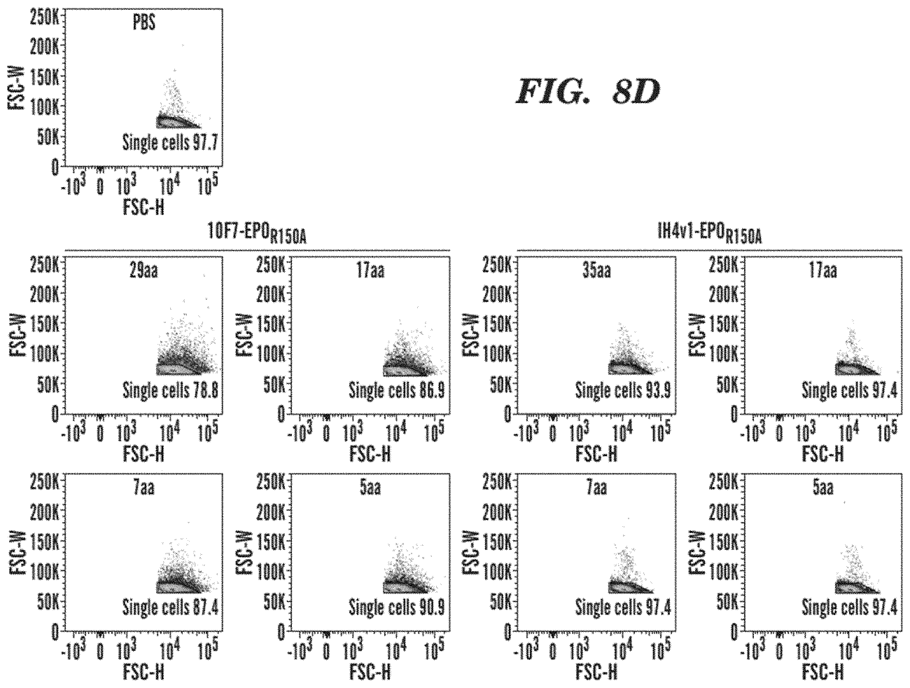

FIGS. 7A-7D demonstrate that the longer linkers resulted in larger shifts in fluorescence intensity peaks, indicating that the longer linkers placed RBC-bound EPO farther away from the cell surface so that the antibody could detect it more easily. In contrast, the IH4v1-EPO R150A fusion proteins containing 5-aa- or 7-aa-long linkers had smaller shifts because RBC-bound EPO was hidden under GYPA molecules and made less accessible by the antibody. Given that the distance between the two antigen binding sites of an IgG antibody is about 155 Angstroms and the average distance between the two adjacent GYPA molecules on a mature RBC is about 140 Angstroms, the accessibility of RBC-bound EPO by even bulkier objects such as cells or cell surface receptors would show similar or even higher contrast between the short and long linkers, indicating that the shorter linkers could prevent unwanted trans activation of non-target cell receptors.

### Example 5: Binding Kinetics of Targeted EPO Fusion Proteins

Targeted EPO fusion proteins contain mutated EPO that has weakened binding affinity for EPOR. The mutated EPO of the fusion protein should still exhibit the same binding affinity for EPOR as the corresponding individual unfused mutant, unless the targeting element, IH4v1, binds GYPA and anchors the fusion protein on the surface.

The fusion molecules of the new composition of matter and their unfused counterparts were subjected to kinetics analysis by biolayer interferometry (BLI) using the BLItz system (ForteBio). A Protein A biosensor (ForteBio) was hydrated in PBST (endotoxin-free PBS, 0.02% Tween-20, pH 7.4) for 10 min at RT prior to the measurement. The baseline measurement was made between the biosensor and the blank (PBST). Then the extracellular domain of EPOR N-terminally fused Fc (R&D Systems) was loaded and immobilized on the biosensor. Another baseline measurement was made between the loaded biosensor and the blank. The biosensor was exposed to a targeted EPO fusion protein and then to the blank for the measurement of the association and dissociation, respectively. Varying concentrations of a targeting EPO fusion protein were measured to calculate the on-rate (kₒₙ), off-rate (k_{off}) and equilibrium dissociation constant (K_{D}).

The kₒₙ, k_{off} and K_{D} of wild-type EPO (EPO WT) were similar to those of EPO WT fused to IH4v1 and the 5-aa linker (IH4v1-5aa-EPO WT), as shown in Table 1. EPO with the Arg150Ala mutation (EPO R150A) had higher k_{off}, and therefore, higher K_{D}, compared to EPO WT. IH4v1-5aa-EPO R150A had similar kₒₙ, k_{off} and K_{D} as EPO R150A. This indicated that the Arg150Ala mutation successfully decreased the binding affinity of EPO for EPOR by making it dissociate faster and that fusing EPO to IH4v1 did not improve the binding kinetics and affinity for EPOR in the absence of GYPA.

### Example 6: Quantitative Illustration of Mechanism and Optimization of Targeted EPO Fusion Proteins

Targeted EPO is engineered to eliminate non-target cell interaction by mutating EPO and limiting the linker length, and to maintain target cell interaction by increasing the local concentration EPO on the target cell surface. The targeting element, an anti-GYPA antibody, anchors targeted EPO fusion protein onto the cell surface, and the flexible glycine-serine linker allows for translational and rotational degrees of freedom of EPO within the limited volume of space defined by the linker length. Therefore, the local effective concentration of EPO is determined by the amount of bound anti-GYPA antibody at the equilibrium and the length of the glycine-serine linker.

The following is an exemplary estimation of the effective concentration of IH4v1-5aa-EPO R150A on a RBC precursor. There are about 50,000 GYPA molecules on a RBC precursor. The equilibrium dissociation constant (K_{D}), or binding affinity, of IH4v1 to GYPA is about 33 nM. Assuming that there is about 1 nM of targeted EPO fusion protein in the tissue extracellular space and that the amount of bound GYPA is small enough to approximate the amount of total GYPA with that of free GYPA, about 3% of the total GYPA would be bound by IH4v1-5aa-EPO R150A. The amount of IH4v1-5aa-EPO R150A can be estimated by multiplying the amount of GYPA molecules and the percent occupancy, and is about 1500 molecules, or 249.1×10⁻²³ moles. The surface area of an RBC precursor is about 1000 um². The length of a 5-aa-long linker is about 15 Angstroms and the binding epitope of IH4v1 on GYPA is about 22.5 Angstroms from the cell surface, so that GYPA-bound EPO can move about 15 Angstroms up and down from the GYPA binding site, resulting in about 30 Angstroms of free movement vertically from the cell surface. The volume around the cell that EPO can occupy can be estimated by multiplying the surface area and the longest distance of EPO from the cell surface, and is about 3 um³, or 3×10⁻⁵ L, in this case. Taken together, the concentration of IH4v1-5aa-EPO R150A around the cell surface is about 830.3 nM, assuming that 1 nM of it gets to the bone marrow extracellular space.

A targeted EPO fusion protein comprising a different anti-GYPA antibody and a linker of different length would result in a different effective concentration. For example, 10F7MN-35aa-EPO R150A is composed of an anti-GYPA antibody, which has K_{D} of about 100 nM, and therefore, would occupy 1% of the total GYPA on a RBC precursor. Switching from IH4v1 to 10F7MN results in a three-fold increase in K_{D}, which leads to a three-fold decrease in the amount of GYPA-bound EPO. At the same time, 10F7MN-35aa-EPO R150A has a linker composed of 35 amino acids, which is about 105 Angstroms in length, and binds GYPA at about 45 Angstroms from the cell surface, so that GYPA-bound EPO can move up to about 150 Angstroms from the cell surface. A longer linker allows for higher spatial degrees of freedom for GYPA-bound EPO, and therefore, increases the volume around the cell. In this case, the volume increased by five fold because the distance that GYPA-bound EPO can move from the cell surface increased by five fold. Overall, the effective concentration of 10F7MN-35aa-EPO R150A around the cell surface decreases by 15-fold compared to IH4v1-5aa-EPO R150A because the effects of the anti-GYPA antibody and the linker length act together multiplicatively.

### Example 7: In Vivo Efficacy of Targeted EPO Fusion Proteins

The targeted EPO fusion proteins comprising IH4v1, EPO R150A and a 5-aa or 35-aa linker were tested in mice for their in vivo efficacy on target cells as well as on non-target cells. The mice used in this assay were transgenic for human GYPA because GYPA proteins in humans and mice are not conserved and have very different sequences. The fusion proteins used in this assay were produced by either CHO-S or CHO DG44 cell lines to ensure sufficient sialylation at the N-linked glycosylation sites of EPO. The number of reticulocytes, or new-born red blood cells, was taken for measuring target cell efficacy, and the number of reticulated platelets, or new-born red blood cells, was taken for measuring non-target cell efficacy.

Five mice received a single intraperitoneal (i.p.) injection with 2 ug of a targeted EPO fusion protein in a 200 uL volume (diluted in saline). 1-5 uL of whole blood was collected by tail-nick in EDTA-coated tubes on days 0, 4, and 7 post-injection. Blood was analyzed immediately after collection by flow cytometry.

Thiazole orange (Sigma-Aldrich) was used to stain reticulocytes and reticulated platelets, and anti-CD41-PE antibody (BD Pharmingen) was used to stain total platelets. A stock solution (1 mg/mL) of thiazole orange was prepared in 100% methanol and was diluted 1:5,000 in PBS to make a 2× working solution. Anti-CD41-PE antibody was diluted 1:500 in the 2× working solution of thiazole orange. 2 uL of whole blood was diluted 1:1,000 in 2 mL of endotoxin-free PBS. Equal volumes (500 uL) of 2× working solution of thiazole orange (containing anti-CD41-PE antibody) and diluted whole blood were mixed and incubated for 30 min in the dark at RT. Then 200 uL of stained blood per sample was added to a 96-well U-bottom plate for flow cytometry. The fluorescence was measured on a LSRFortessa SORP flow cytometer equipped with an optional HTS sampler (BD Biosciences) using the following filter configuration: PE excitation, 561/50 mW; emission filter, BP 582/15; YFP excitation, 488/100 mW; emission filter.

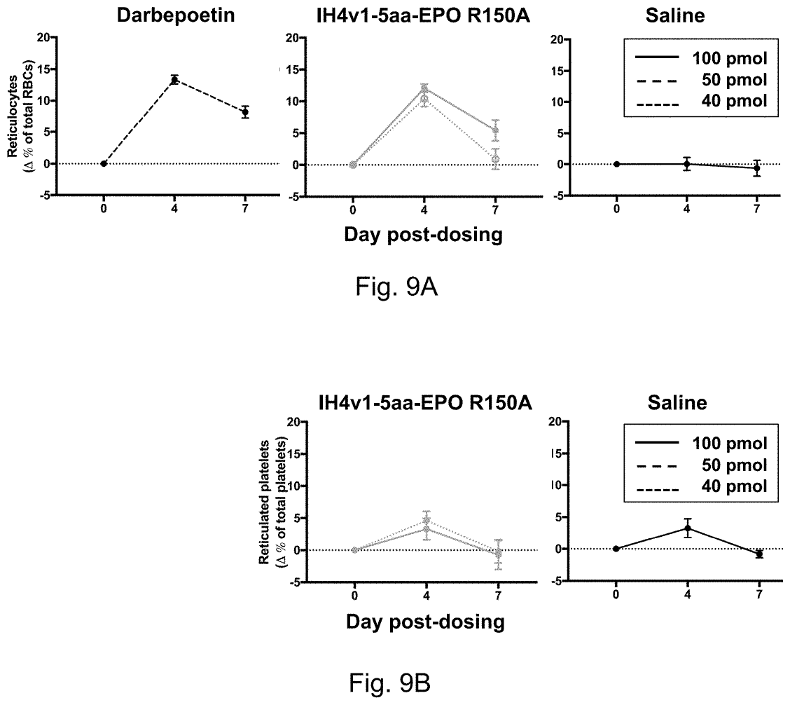

FIG. 9A shows that 40 pmol of IH4v1-5aa-EPO R150A increased the reticulocyte count by about 12% four days after injection. The effects were comparable to those of 50 pmol of Darbepoetin (hyperglycosylated wild-type EPO; Aranesp, Amgen) and 125 pmol of the previous targeted EPO fusion protein (10F7-35aa-EPO R150A). The same doses of IH4v1-5aa-EPO R150A did not change the reticulated platelet count (FIG. 9B), in contrast to Darbepoetin, which increased the reticulated platelet count by about 12%. This demonstrates that the new targeted EPO fusion protein, IH4v1-5aa-EPO R150A, has similar or higher efficacy as Darbepoetin and successfully eliminates undesired non-target cell activity. This also suggests that IH4v1-5aa-EPO R150A would have a lower minimal effective dose (MED) than the current EPO products, such as Epogen™ (Amgen) and Aranesp™ (Amgen).

### Example 8: Comparison of Thrombotic Side Effects Induced by Targeted EPO Fusion Proteins and Other EPO Variants and Products In Vivo

The targeted EPO fusion protein, IH4v1-5aa-EPO R150A, was tested for thrombotic side effects in huGYPA-transgenic mice. The fusion proteins used in this assay were produced by either CHO-S or CHO DG44 cell lines and contained sialic acids at the N-linked glycosylation sites. Thrombotic side effects were studied at a systemic level by measuring mouse tail bleeding time.

Ten mice received a single intraperitoneal (i.p.) injection with 2 ug of a targeted EPO fusion protein in a 200 uL volume (diluted in saline). 24 hours after injection, the mice were anaesthetized. Then, the tips of their tails were cut 3 mm from the end and dipped in 37° C. saline (0-2 mm from the air-saline surface. The bleeding time was measured up to 600 seconds. No noticeable tail bleeding for 60 seconds was considered as the end of the bleeding time.

As shown in FIGS. 10A-10B, the mice injected with IH4v1-5aa-EPO R150A had similar bleeding time as those injected with saline. In contrast, the mice injected with Darbepoetin (Aranesp, Amgen) or 10F7MN-35aa-EPO R150A at a dose that gave similar efficacy as 2 ug of targeted EPO had shortened bleeding time compared to the saline control. Interestingly, the mice injected with IH4v1-EPO R150A containing a 35-aa linker still showed shortened bleeding time although they showed slightly longer bleeding time compared to 10F7MN-35aa-EPO R150A. This suggests that IH4v1 is a better targeting element than 10F7MN but needs a short linker to synergistically eliminate the thrombotic side effects of EPO.

### Example 9: Comparison of Platelet Activation States Induced by Targeted EPO Fusion Proteins and Other EPO Products In Vivo

EPO increases activated platelets by either producing hyper-reactive platelets or stimulating platelet activation via an unknown mechanism. Targeted EPO fusion proteins were shown not to stimulate the production of platelets (FIG. 10B) and are engineered to eliminate non-target cell interaction so that only the desired erythropoietic effect would be achieved. Their effects on platelet activation states in vivo can be studied by detecting P-selectin (CD62P) or integrin αIIbβ3 (GP IIb/IIIa; CD41/61) exposed on the surface of platelets, or by measuring the expression levels of ADP receptors, P2Y₁ and/or P2Y₁₂ in platelets.

Transgenic mice expressing human GYPA would receive a single i.p. injection with 2-5 ug of a targeted EPO fusion protein in a 200 uL volume (diluted in saline). 24 hours after injection, the mice would be anaesthetized and 200 uL of whole blood would be collected by a submandibular bleed. The blood may be kept and transported to the lab at RT for further processing.

For the detection of activated platelet surface markers, such as P-selectin and integrin αIIbβ3, standard flow cytometry may be used. Whole blood is diluted 1:25 in the modified HEPES-Tyrode's buffer (137 mM NaCl, 2 mM KCl, 0.3 mM Na₂HPO₄, 12 mM NaHCO₃, 1 mM MgSO₄, 5 mM glucose, 5 mM HEPES, pH 6.8) and co-stained with anti-mouse P-selectin-FITC antibody (clone Wug.E9, Emfret Analytics) and anti-mouse integrin αIIbβ3-PE antibody (clone Jon/A, Emfret Analytics). 5 uL of each antibody per uL of whole blood is added to the diluted blood. After 15 minutes of incubation at RT, 400 uL of PBS is added to the stained blood to stop the reaction. Total platelets can be distinguished from other blood cell populations by FSC and SSC. Platelets with cell surface P-selectin and integrin αIIbβ3 can be detected by FITC and PE, respectively.

Blood from mice treated with a current EPO product would result in high levels of P-selectin- and integrin αIIbβ3-positive platelets, indicating that alpha granules, containing P-selectin, have been translocated and that integrins αIIb and β3 have paired up to form an active conformation, all of which are the signs of platelet activation. In contrast, mice treated with a targeted EPO fusion protein would result in baseline levels of P-selectin- and integrin αIIbβ3-positive cells.

For the measurement of the expression levels of ADP receptors, such as P2Y₁ and P2Y₁₂, Western blotting may be used. Whole blood is spun down at 150×g for 15 min at 22° C. in a swinging-bucket centrifuge without brake. Plasma and buffy coat are collected and spun down at 200×g for 10 min at 22° C. without brake. Supernatant is collected and 0.5 uM of PGI₂ is added to prevent further platelet activation. Then the supernatant is spun down at 2,000×g for 5 min at 22° C. without brake. Supernatant is discarded and the pellet (platelets) is resuspended in 100 uL of washing buffer (129 mM NaCl, 13.6 mM sodium citrate, 1.6 mM KH₂PO₄, 11.1 mM glucose, 0.5 uM PGI₂, pH 6.8). The resuspended platelets are spun down again at 2,000×g for 5 min at 22° C. without brake. Then the pellet (washed platelets) is resuspended in 100 uL of ice-cold lysis buffer (1% NP-40, 0.1% SDS, 20 mM Tris-HCl pH 8.0, 137 mM NaCl, 2 mM EDTA, 10% glycerol, pH 7.5) and let sit on ice for 30 min with periodic mixing. The lysates are spun down at 2,000×g for 7 min at 4° C. Then the supernatant can be run on an SDS-PAGE gel and analyzed by Western blotting. Blots can be probed with anti-mouse P2Y₁ antibody (Santa Cruz) and anti-mouse P2Y₁₂ antibody (Abcam). Anti-mouse CD41 antibody (Abcam) can be used as a loading control.

In mice treated with a current EPO product, the expression of both P2Y₁ and P2Y₁₂ would increase as a result of platelet activation. In contrast, those treated with a targeted EPO fusion protein would have baseline levels of P2Y₁ and P2Y₁₂, similarly to the saline-treated mice.

### Example 10: Engineering Protein Production Cell Lines

To produce a homogeneous batch of proteins in a large quantity, a monoclonal cell line that stably expresses the properly glycosylated form of a targeted EPO fusion protein was generated. CHO DG44 cells were seeded in CD DG44 medium (Gibco™) supplemented with GlutaMax™ (Gibco™) to yield 8 mM final concentration of L-glutamine and 0.18% Pluronic™ F-68 (Gibco™). They were transfected with plasmids comprising the pOptiVEC™ vector and the targeted EPO, using the FreeStyle™ MAX reagent, and incubated in a shaker at 37° C., 5% CO₂ for 48-72 hr after transfection. Then the cells were transferred to hypoxanthine- and thymidine-deficient CD OptiCHO medium (Gibco™) supplemented with GlutaMax™ to yield 6 mM final concentration of L-glutamine and passaged until the viability reached >90% to select for the transfected cells. The stable pool of transfected cells was subjected to clonal selection by limiting dilution to obtain monoclonality. The cells were seeded on a 96-well plate at 1 cell per well and let grow for 10-14 days in an incubator at 37° C., 5% CO₂, without shaking. Clonal growth was visually inspected on an inverted light microscope. The grown clones were scaled up to the next larger plate or flask every 3-7 days. The supernatant of each clone was collected and assayed for the expression of the desired targeted EPO fusion protein by Western blotting. The monoclonal cell lines with high expression levels of targeted EPO were frozen in the complete CD OptiCHO medium supplemented with 10% DMSO and stored in the liquid nitrogen tank.

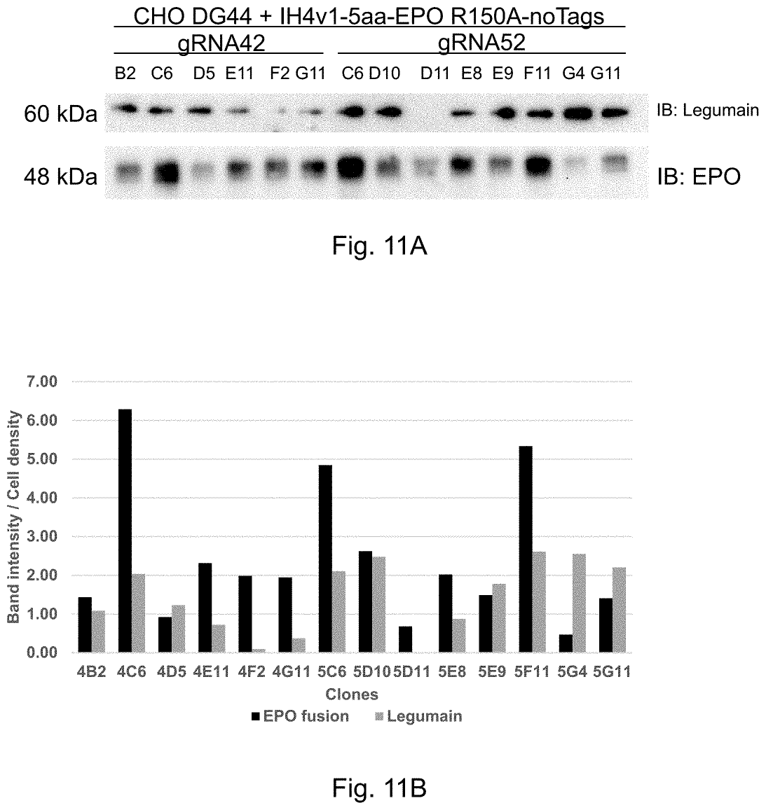

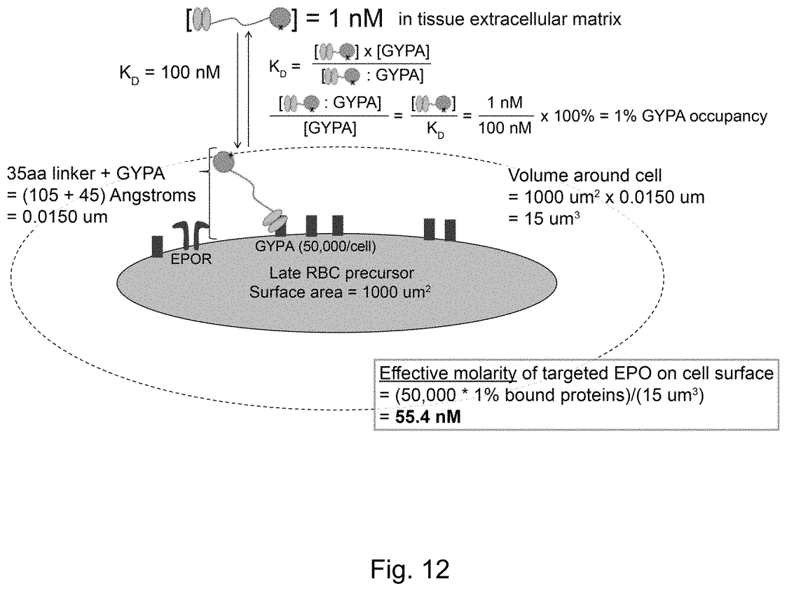

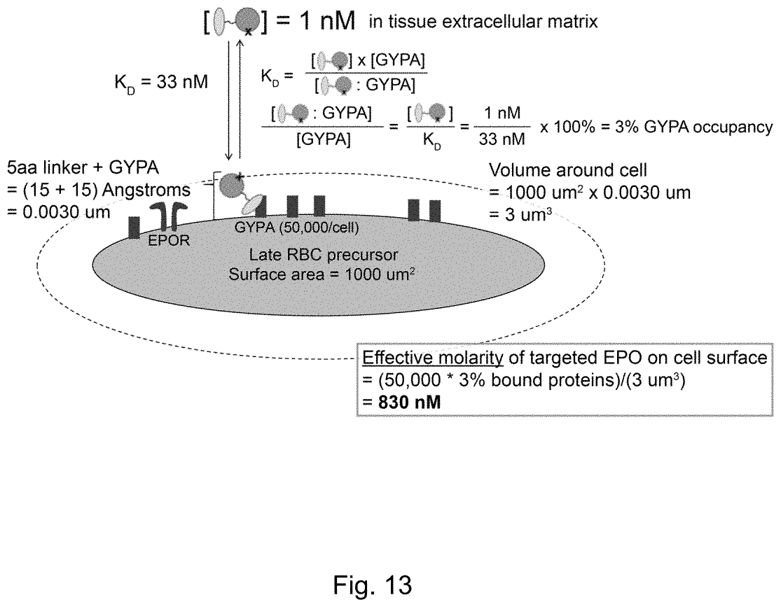

Targeted EPO fusion proteins produced by CHO DG44 cells were often contaminated by one of the host cell proteins called legumain, an asparaginyl endopeptidase. About 30-40% and 5% of the total protein purified by the nickel and cobalt resins, respectively, was legumain. Therefore, the legumain gene was knocked out in the production cell lines by using the CRISPR-Cas9 system. The CRISPR RNA (crRNA) sequences targeting the Chinese Hamster legumain gene were designed using the online resource, CRISPy-web. The crRNA and trans-activating crRNA (tracrRNA) molecules were ordered from Integrated DNA Technologies (IDT™). A pair of crRNA and tracrRNA was duplexed to create a single guide RNA (sgRNA) molecule by heating at 95° C. for 5 min and cooling to RT. The sgRNA and Cas9 protein (GeneArt™ Platinum™ Cas9 nuclease, Invitrogen™) were transfected into a monoclonal production cell line using the Lipofectamine™ CRISPRMAX™ reagent (Invitrogen™). 48-72 hours after transfection, the cells were seeded on a 96-well plate at 1 cell per well for clonal selection by limiting dilution and let sit for 10-14 days in an incubator at 37° C., 5% CO₂. The clones with visual signs of growth were scaled up to the next larger plate or flask every 3-7 days. A small fraction of each clone was harvested to check for knockout. The genomic DNA was extracted from the cells and the sgRNA target region was analyzed for an insertion or a deletion (indel) using Sanger sequencing. Also, the amount of secreted legumain in the supernatant was measured by Western blotting. As shown in FIGS. 11A-11B, 14 clones expressed IH4v1-5aa-EPO R150A (without tags) and one of them, 5D11, acquired an insertion at the Cas9 cut site at the exon 5 of the legumain gene, resulting in the lack of expression of legumain.

```
IH4
SEQ ID NO: 1
CAGGTCCAACTGCAGGAGAGCGGCGGGGGGTCAGTTCAGGCGGGGGGGAGTCTGCGGTTGAGCTGCGT
AGCTTCAGGCTACACTGACAGCACCTACTGCGTGGGATGGTTTCGGCAGGCACCCGGCAAGGAACGAG
AGGGCGTTGCACGGATCAACACTATCTCCGGTCGGCCTTGGTACGCAGATAGTGTTAAGGGACGGTTT
ACTATTAGTCAGGATAACTCTAAGAATACCGTCTACCTTCAGATGAATAGCCTGAAACCGGAAGACAC
GGCTATTTACTATTGCACCCTTACAACTGCCAACAGCAGAGGGTTTTGTTCTGGGGGATATAACTACA
AAGGACAGGGGCAAGTCACTGTCAGC
IH4v1 (v1 mutation appears at bp 352-354, shown in bold)
SEQ ID NO: 2
CAGGTCCAACTGCAGGAGAGCGGCGGGGGGTCAGTTCAGGCGGGGGGGAGTCTGCGGTTGAGCTGCGT
AGCTTCAGGCTACACTGACAGCACCTACTGCGTGGGATGGTTTCGGCAGGCACCCGGCAAGGAACGAG
AGGGCGTTGCACGGATCAACACTATCTCCGGTCGGCCTTGGTACGCAGATAGTGTTAAGGGACGGTTT
ACTATTAGTCAGGATAACTCTAAGAATACCGTCTACCTTCAGATGAATAGCCTGAAACCGGAAGACAC
GGCTATTTACTATTGCACCCTTACAACTGCCAACAGCAGAGGGTTTTGTTCTGGGGGATATAACTACA
AAGGACAGGGGACCCAAGTCACTGTCAGC
5 aa Gly-Ser linker
SEQ ID NO: 3
TCTGGTGGTGGTTCC
7 aa Gly-Ser linker
SEQ ID NO: 4
GGAGGATCTGGTGGTGGTTCC
17 aa Gly-Ser linker
SEQ ID NO: 5
GGAGGATCCGGTGGTGGAGGATCATCTGGTGGAGGATCTGGTGGTGGTTCC
35 aa Gly-Ser linker
SEQ ID NO: 6
GGTGGAGGTGGTTCCGGAGGAGGAAGTTCCGGTGGTGGATCTTCTTCTGGAGGTGGAGGATCCGGTGG
TGGAGGATCATCTGGTGGAGGATCTGGTGGTGGTTCC
EPO wt (R150 residue encoded by bp 448-450, shown in bold)
SEQ ID NO: 7
GCTCCACCTAGATTGATTTGTGATTCCAGAGTTTTGGAAAGATACTTGTTGGAAGCTAAGGAGGCTGA
AAATATTACTACTGGTTGTGCTGAACATTGTTCTTTGAACGAGAATATTACTGTTCCAGATACTAAGG
TTAACTTTTACGCTTGGAAGAGAATGGAAGTTGGTCAGCAAGCTGTTGAAGTTTGGCAAGGTTTGGCT
TTGTTGTCTGAAGCTGTTTTGAGAGGTCAAGCTTTGTTGGTTAATTCTTCTCAACCATGGGAACCATT
GCAATTGCATGTTGATAAGGCTGTTTCTGGTTTGAGATCTTTGACTACCTTGTTGAGAGCTTTGGGTG
CTCAAAAGGAAGCTATTTCTCCTCCAGATGCTGCTTCTGCCGCTCCATTGAGAACTATTACTGCTGAT
ACTTTTAGAAAGTTGTTTAGAGTTTACTCTAACTTCTTGAGAGGTAAGTTGAAGTTGTACACTGGTGA
AGCTTGTAGAACTGGTGATCGG
EPO R150A (R150A mutation appears at bp 448-450, shown in bold)
SEQ ID NO: 8
GCTCCACCTAGATTGATTTGTGATTCCAGAGTTTTGGAAAGATACTTGTTGGAAGCTAAGGAGGCTGA
AAATATTACTACTGGTTGTGCTGAACATTGTTCTTTGAACGAGAATATTACTGTTCCAGATACTAAGG
TTAACTTTTACGCTTGGAAGAGAATGGAAGTTGGTCAGCAAGCTGTTGAAGTTTGGCAAGGTTTGGCT
TTGTTGTCTGAAGCTGTTTTGAGAGGTCAAGCTTTGTTGGTTAATTCTTCTCAACCATGGGAACCATT
GCAATTGCATGTTGATAAGGCTGTTTCTGGTTTGAGATCTTTGACTACCTTGTTGAGAGCTTTGGGTG
CTCAAAAGGAAGCTATTTCTCCTCCAGATGCTGCTTCTGCCGCTCCATTGAGAACTATTACTGCTGAT
ACTTTTAGAAAGTTGTTTAGAGTTTACTCTAACTTCTTGGCCGGTAAGTTGAAGTTGTACACTGGTGA
AGCTTGTAGAACTGGTGATCGG
depicts C-terminal epitope tags
SEQ ID NO: 9
CGGGCCCGAACAAAAACTCATCTCAGAAGAGGATCTGAATAGCGCCGTCGACCATCATCATCATCATC
AT
IH4
SEQ ID NO: 10
QVQLQESGGGSVQAGGSLRLSCVASGYTDSTYCVGWFRQAPGKEREGVARINTISGRPWYADSVKGRF
TISQDNSKNTVYLQMNSLKPEDTAIYYCTLTTANSRGFCSGGYNYKGQGQVTVS
IH4v1 (v1 variation occurs at residue 118, in bold)
SEQ ID NO: 11
QVQLQESGGGSVQAGGSLRLSCVASGYTDSTYCVGWFRQAPGKEREGVARINTISGRPWYADSVKGRF
TISQDNSKNIVYLQMNSLKPEDTAIYYCTLTTANSRGFCSGGYNYKGQGTQVTVS
5 aa Gly-Ser linker
SEQ ID NO: 12
SGGGS
7 aa Gly-Ser linker
SEQ ID NO: 13
GGSGGGS
17 aa Gly-Ser linker
SEQ ID NO: 14
GGSGGGGSSGGGSGGGS
35 aa Gly-Ser linker
SEQ ID NO: 15
GGGGSGGGSSGGGSSSGGGGSGGGGSSGGGSGGGS
EPO wt
SEQ ID NO: 16
APPRLICDSRVLERYLLEAKEAENITTGCAEHCSLNENITVPDTKVNFYAWKRMEVGQQAVEVWQGLA
LLSEAVLRGQALLVNSSQPWEPLQLHVDKAVSGLRSLTTLLRALGAQKEAISPPDAASAAPLRTITAD
TFRKLFRVYSNFLRGKLKLYTGEACRTGDR
EPO R150A
SEQ ID NO: 17
APPRLICDSRVLERYLLEAKEAENITTGCAEHCSLNENITVPDTKVNFYAWKRMEVGQQAVEVWQGLA
LLSEAVLRGQALLVNSSQPWEPLQLHVDKAVSGLRSLTTLLRALGAQKEAISPPDAASAAPLRTITAD
TFRKLFRVYSNFLAGKLKLYTGEACRTGDR
C-terminal epitope tags
SEQ ID NO: 18
GPEQKLISEEDLNSAVDHHHHHH
18 aa linker
SEQ ID NO: 19
GGTGGAGGTGGATCTGGTGGTGGAGGATCTTCAGGAGGTGGTGGATCTTCC
18 aa linker
SEQ ID NO: 20
GGGGSGGGGSSGGGGSS
29 aa linker
SEQ ID NO: 21
GGAGGAAGTTCCGGTGGTGGATCTTCTTCTGGAGGTGGAGGATCCGGTGGTGGAGGATCATCTGGTGG
AGGATCTGGTGGTGGTTCC
29 aa linker
SEQ ID NO: 22
GGSSGGGSSSGGGGSGGGGSSGGGSGGGS
39 aa linker
SEQ ID NO: 23
GGTGGAAGTAGTGGTGGAGGTGGTTCCGGAGGAGGAAGTTCCGGTGGTGGATCTTCTTCTGGAGGTGGAGG
ATCCGGTGGTGGAGGATCATCTGGTGGAGGATCTGGTGGTGGTTCC
39 aa linker
SEQ ID NO: 24
GGSSGGGGSGGGSSGGGSSSGGGGSGGGGSSGGGSGGGS
epitope of GYPA bound by IH4
SEQ ID NO: 25
YPPE
human GYPA (e. g., NCBI Ref Seq, NP_002090.4)
SEQ ID NO: 26
    1 mygkiifvll lseivsisal sttevamhts tsssvtksyi ssqtndthkr dtyaatprah
  61 evseisvrtv yppeeetger vqlahhfsep eitliifgvm agvigtilli sygirrlikk
121 spsdvkplps pdtdvpIssy eienpetsdq
CDR1 of IH4
SEQ ID NO: 27
SGYTDSTYCVG
CDR2 of IH4
SEQ ID NO: 28
RINTISGRPWYADSVKG
CDR3 of IH4
SEQ ID NO: 29
TTANSRGFCSGGYNY
```

SEQ ID NO: 31 provides the nucleic acid sequence of IH4v1-5aa-EPO R150A-cMyc-6×His in pSecTag2A vector. The components of the sequence are: CMV promoter (bases 208-861); T7 promoter (bases 862-881); Kozak sequence (gcc(G/A)ccATGG) (bases 898-907); Murine IgK secretion tag (leader sequence) (bases 904-963); IH4v1 nanobody (bases 964-1332); 5aa linker (bases 1333-1347); EPO R150A (bases 1348-1844); cMyc tag (bases 1851-1881); 6×His tag (bases 1897-1914); BGH polyadenylation sequence (bases 1935-2053); F1 origin (bases 2213-2626); SV40 promoter & origin (bases 2694-3015); EM7 promoter (bases 3031-3097); Zeocin™ resistance gene (bases 3098-3472); SV40 polyadenylation sequence (bases 3602-3732); pUC origin (bases 4115-4788); Ampicillin resistance gene (bases 4933-5793); and Ampicillin (bla) promoter (bases 5788-5892)

SEQ ID NO: 31
ACGGATCGGGAGATCTCCCGATCCCCTATGGTCGACTCTCAGTACAATCTGCTCTGATGCCGCATAGT
TAAGCCAGTATCTGCTCCCTGCTTGTGTGTTGGAGGTCGCTGAGTAGTGCGCGAGCAAAATTTAAGCT
ACAACAAGGCAAGGCTTGACCGACAATTGCATGAAGAATCTGCTTAGGGTTAGGCGTTTTGCGCTGCT
TCGCGATGTACGGGCCAGATATACGCGTTGACATTGATTATTGACTAGTTATTAATAGTAATCAATTA
CGGGGTCATTAGTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATAGTAACGCCAAT
AGGGACTTTCCATTGACGTCAATGGGTGGACTATTTACGGTAAACTGCCCACTTGGCAGTACATCAAG
TGTATCATATGCCAAGTACGCCCCCTATTGACGTCAATGACGGTAAATGGCCCGCCTGGCATTATGCC
CAGTACATGACCTTATGGGACTTTCCTACTTGGCAGTACATCTACGTATTAGTCATCGCTATTACCAT
GGTGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTCGT
AACAACTCCGCCCCATTGACGCAAATGGGCGGTAGGCGTGTACGGTGGGAGGTCTATATAAGCAGAGC
TCTCTGGCTAACTAGAGAACCCACTGCTTACTGGCTTATCGAAATTAATACGACTCACTATAGGGAGA
CCCAAGCTGGCTAGCCACCATGGAGACAGACACACTCCTGCTATGGGTACTGCTGCTCTGGGTTCCAG
GTTCCACTGGTCAGGTCCAACTGCAGGAGAGCGGCGGGGGGTCAGTTCAGGCGGGGGGGAGTCTGCGG
TTGAGCTGCGTAGCTTCAGGCTACACTGACAGCACCTACTGCGTGGGATGGTTTCGGCAGGCACCCGG
CAAGGAACGAGAGGGCGTTGCACGGATCAACACTATCTCCGGTCGGCCTTGGTACGCAGATAGTGTTA
AGGGACGGTTTACTATTAGTCAGGATAACTCTAAGAATACCGTCTACCTTCAGATGAATAGCCTGAAA
CCGGAAGACACGGCTATTTACTATTGCACCCTTACAACTGCCAACAGCAGAGGGTTTTGTTCTGGGGG
ATATAACTACAAAGGACAGGGGACCCAAGTCACTGTCAGCTCTGGTGGTGGTTCCGCTCCACCTAGAT
TGATTTGTGATTCCAGAGTTTTGGAAAGATACTTGTTGGAAGCTAAGGAGGCTGAAAATATTACTACT
GGTTGTGCTGAACATTGTTCTTTGAACGAGAATATTACTGTTCCAGATACTAAGGTTAACTTTTACGC
TTGGAAGAGAATGGAAGTTGGTCAGCAAGCTGTTGAAGTTTGGCAAGGTTTGGCTTTGTTGTCTGAAG
CTGTTTTGAGAGGTCAAGCTTTGTTGGTTAATTCTTCTCAACCATGGGAACCATTGCAATTGCATGTT
GATAAGGCTGTTTCTGGTTTGAGATCTTTGACTACCTTGTTGAGAGCTTTGGGTGCTCAAAAGGAAGC
TATTTCTCCTCCAGATGCTGCTTCTGCCGCTCCATTGAGAACTATTACTGCTGATACTTTTAGAAAGT
TGTTTAGAGTTTACTCTAACTTCTTGGCCGGTAAGTTGAAGTTGTACACTGGTGAAGCTTGTAGAACT
GGTGATCGGGGGCCCGAACAAAAACTCATCTCAGAAGAGGATCTGAATAGCGCCGTCGACCATCATCA
TCATCATCATTGAGTTTAAACCCGCTGATCAGCCTCGACTGTGCCTTCTAGTTGCCAGCCATCTGTTG
TTTGCCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGGACAG
CAAGGGGGAGGATTGGGAAGACAATAGCAGGCATGCTGGGGATGCGGTGGGCTCTATGGCTTCTGAGG
CGGAAAGAACCAGCTGGGGCTCTAGGGGGTATCCCCACGCGCCCTGTAGCGGCGCATTAAGCGCGGCG
GGTGTGGTGGTTACGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGGGCATCCCTTTAG
GGTTCCGATTTAGTGCTTTACGGCACCTCGACCCCAAAAAACTTGATTAGGGTGATGGTTCACGTAGT
GGGCCATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTTTGG
GGATTTCGGCCTATTGGTTAAAAAATGAGCTGATTTAACAAAAATTTAACGCGAATTAATTCTGTGGA
ATGTGTGTCAGTTAGGGTGTGGAAAGTCCCCAGGCTCCCCAGCAGGCAGAAGTATGCAAAGCATGCAT
CTCAATTAGTCAGCAACCAGGTGTGGAAAGTCCCCAGGCTCCCCAGCAGGCAGAAGTATGCAAAGCAT
GCATCTCAATTAGTCAGCAACCATAGTCCCGCCCCTAACTCCGCCCATCCCGCCCCTAACTCCGCCCA
GTTCCGCCCATTCTCCGCCCCATGGCTGACTAATTTTTTTTATTTATGCAGAGGCCGAGGCCGCCTCT
GCCTCTGAGCTATTCCAGAAGTAGTGAGGAGGCTTTTTTGGAGGCCTAGGCTTTTGCAAAAAGCTCCC
GGGAGCTTGTATATCCATTTTCGGATCTGATCAGCACGTGTTGACAATTAATCATCGGCATAGTATAT
CGGCATAGTATAATACGACAAGGTGAGGAACTAAACCATGGCCAAGTTGACCAGTGCCGTTCCGGTGC
TCACCGCGCGCGACGTCGCCGGAGCGGTCGAGTTCTGGACCGACCGGCTCGGGTTCTCCCGGGACTTC
GTGGAGGACGACTTCGCCGGTGTGGTCCGGGACGACGTGACCCTGTTCATCAGCGCGGTCCAGGACCA
GGTGGTGCCGGACAACACCCTGGCCTGGGTGTGGGTGCGCGGCCTGGACGAGCTGTACGCCGAGTGGT
CGGAGGTCGTGTCCACGAACTTCCGGGACGCCTCCGGGCCGGCCATGACCGAGATCGGCGAGCAGCCG
TGGGGGCGGGAGTTCGCCCTGCGCGACCCGGCCGGCAACTGCGTGCACTTCGTGGCCGAGGAGCAGGA
CTGACACGTGCTACGAGATTTCGATTCCACCGCCGCCTTCTATGAAAGGTTGGGCTTCGGAATCGTTT
TCCGGGACGCCGGCTGGATGATCCTCCAGCGCGGGGATCTCATGCTGGAGTTCTTCGCCCACCCCAAC
TTGTTTATTGCAGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTATCATGTCTGTATACCGT
CGACCTCTAGCTAGAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACCTGTCGTGCCAGCTGCATT
AATGAATCGGCCAACGCGCGGGGAGAGGCGGTTTGCGTATTGGGCGCTCTTCCGCTTCCTCGCTCACT
GACTCGCTGCGCTCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAA
GCGTGGCGCTTTCTCAATGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGGACAGTATTT
GGTATCTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATGAAGTTTTAAATC
AATCTAAAGTATATATGAGTAAACTTGGTCTGACAGTTACCAATGCTTAATCAGTGAGGCACCTATCT
CAGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACTGCATAATTCT
CTTACTGTCATGCCATCCGTAPGATGCTTTTCTGTGACTGGTGAGTACTCAPCCAPGTCATTCTGAGA
ATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCCCGAAAAGT
GCCACCTGACGTCG

SEQ ID NO: 32 provides the nucleic acid sequence of IH4v1-5aa-EPO R150A-cMyc-6×His in pOptiVEC vector. The components are: CMV promoter (bases 36-715); T7 promoter (bases 731-750); Kozak sequence (gcc(G/A)ccATGG)(bases 767-776); Murine IgK secretion tag (leader sequence)(bases 773-832); IH4v1 nanobody (bases 833-1201); 5aa linker (bases 1202-1216); EPO R150A (bases 1217-1714); cMyc tag (bases 1721-1753); 6×His tag (bases 1790-1807); EMCV IRES (bases 1865-2454); DHFR (bases 2467-3030); TK polyadenylation signal (bases 3070-3342); pUC origin (bases 3704-4377); Ampicillin resistance gene (bases 4519-5379); and Ampicillin (bla) promoter (bases 5374-5478)

SEQ ID NO: 32
AAAGTGCCACCTGACGTCGACGGATCGGGAGATCAGTTGACATTGATTATTGACTAGTTATTAATAGT
AATCAATTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGAT
TTCCAAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGGTGGGAGGTCTATAT
AAGCAGAGCTCGTTTAGTGAACCGTCAGATCGCCTGGAGACGCCATCCACGCTGTTTTGACCTCCATA
GAAGACACCGGGACCGATCCAGCCTCCGGACTCTAGAGGATCCAACCCTTTAATACGACTCACTATAG
GGAGACCCAAGCTGGCTAGCCACCATGGAGACAGACACACTCCTGCTATGGGTACTGCTGCTCTGGGT
TCCAGGTTCCACTGGTCAGGTCCAACTGCAAGAGAGCGGCGGGGGGTCAGTTCAGGCGGGGGGGAGTC
TGCGGTTGAGCTGCGTAGCTTCAGGCTACACTGACAGCACCTACTGCGTGGGATGGTTTCGGCAGGCA
CCCGGCAAGGAACGAGAGGGCGTTGCACGGATCAACACTATCTCCGGTCGGCCTTGGTACGCAGATAG
TGTTAAGGGACGGTTTACTATTAGTCAGGATAACTCTAAGAATACCGTCTACCTTCAGATGAATAGCC
TGAAACCGGAAGACACGGCTATTTACTATTGCACCCTTACAACTGCCAACAGCAGAGGGTTTTGTTCT
GGGGGATATAACTACAAAGGACAGGGGACCCAAGTCACTGTCAGCTCTGGTGGTGGTTCCGCTCCACC
TAGATTGATTTGTGATTCCAGAGTTTTGGAAAGATACTTGTTGGAAGCTAAGGAGGCTGAAAATATTA
CTACTGGTTGTGCTGAACATTGTTCTTTGAACGAGAATATTACTGTTCCAGATACTAAGGTTAACTTT
TACGCTTGGAAGAGAATGGAAGTTGGTCAGCAAGCTGTTGAAGTTTGGCAAGGTTTGGCTTTGTTGTC
TGAAGCTGTTTTGAGAGGTCAAGCTTTGTTGGTTAATTCTTCTCAACCATGGGAACCATTGCAATTGC
ATGTTGATAAGGCTGTTTCTGGTTTGAGATCTTTGACTACCTTGTTGAGAGCTTTGGGTGCTCAAAAG
GAAGCTATTTCTCCTCCAGATGCTGCTTCTGCCGCTCCATTGAGAACTATTACTGCTGATACTTTTAG
AAAGTTGTTTAGAGTTTACTCTAACTTCTTGGCCGGTAAGTTGAAGTTGTACACTGGTGAAGCTTGTA
GAACTGGTGATCGGGGGCCCGAACAAAAACTCATCTCAGAAGAGGATCTGAATAGCGCCCTGGTTCCG
AGAGGCTCCCGTCTCGTCGACCATCATCATCATCATCATTGAGTTTAAACCCGCTGAAAGGGTTGGAT
CCCTACCGGTGCTGCGGCCGCGCAGTTAACGCCGCCCCTCTCCCTCCCCCCCCCCTAACGTTACTGGC
CGAAGCCGCTTGGAATAAGGCCGGTGTGCGTTTGTCTATATGTTATTTTCCACCATATTGCCGTCTTT
TGGCAATGTGAGGGCCCGGAAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCCTCTGGAAGCTTCTTGAAGA
CAAACAACGTCTGTAGCGACCCTTTGCAGGCAGCGGAACCCCCCACCTGGCGACAGGTGCCTCTGCGG
CCAAAAGCCACGTGTATAAGATACACCTGCAAAGGCGGCACAACCCCAGTGCCACGTTGTGAGTTGGA
TAGTTGTGGAAAGAGTCAAATGGCTCTCCTCAAGCGTATTCAACAAGGGGCTGAAGGATGCCCAGAAG
GTACCCCATTGTATGGGATCTGATCTGGGGCCTCGGTACACATGCTTTACATGTGTTTAGTCGAGGTT
AAAAAAACGTCTAGGCCCCCCGAACCACGGGGACGTGGTTTTCCTTTGAAAAACACGATGATAATATG
GCCACAAGATCTGCCACCATGGTTCGACCATTGAACTGCATCGTCGCCGTGTCCCAAAATATGGGGAT
TGGCAAGAACGGAGACCTACCCTGGCCTCCGCTCAGGAACGAGTTCAAGTACTTCCAAAGAATGACCA
CAACCTCTTCAGTGGAAGGTAAACAGAATCTGGTGATTATGGGTAGGAAAACCTGGTTCTCCATTCCT
GAGAAGAATCGACCTTTAAAGGACAGAATTAATATAGTTCTCAGTAGAGAACTCAAAGAACCACCACG
AGGAGCTCATTTTCTTGCCAAAAGTTTGGATGATGCCTTAAGACTTATTGAACAACCGGAATTGGCAA
GTAAAGTAGACATGGTTTGGATAGTCGGAGGCAGTTCTGTTTACCAGGAAGCCATGAATCAACCAGGC
CACCTCAGACTCTTTGTGACAAGGATCATGCAGGAATTTGAAAGTGACACGTTTTTCCCAGAAATTGA
TTTGGGGAAATATAAACTTCTCCCAGAATACCCAGGCGTCCTCTCTGAGGTCCAGGAGGAAAAAGGCA
TCAAGTATAAGTTTGAAGTCTACGAGAAGAAAGACTAAAACCGGTTAGTAATGAGTTTAAACGGGGGA
GGCTAACTGAAACACGGAAGGAGACAATACCGGAAGGAACCCGCGCTATGACGGCAATAAAAAGACAG
AATAAAACGCACGGGTGTTGGGTCGTTTGTTCATAAACGCGGGGTTCGGTCCCAGGGCTGGCACTCTG
TCGATACCCCACCGAGACCCCATTGGGGCCAATACGCCCGCGTTTCTTCCTTTTCCCCACCCCACCCC
CCAAGTTCGGGTGAAGGCCCAGGGCTCGCAGCCAACGTCGGGGCGGCAGGCCCTGCCATTACCGTCGA
CCTCTAGCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCAGCTGCATTAAT
GAATCGGCCAACGCGCGGGGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAAGGCGGTAATACGGTTATC
CACAGAATCAGGGGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAAACAAAC
CACCGCTGGTAGCGGTTTTTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGCCACATAGCAGAACTT
TAAAAGTGCTCATCATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGA

### Example 11: Rational Design of a Bifunctional AND-Gate Ligand to Modulate Cell-Cell Interactions

Protein “AND-gate” systems, in which a ligand acts only on cells with two different receptors, can direct signaling activity to a particular cell type and avoid action on other cells. In a bifunctional AND-Gate protein, the molecular geometry of the protein domains is crucial. Described herein is the construction a tissue-targeted erythropoietin (EPO) that stimulates red blood cell production without triggering thrombosis. EPO was directed to red blood cell (RBC) precursors and mature RBCs by fusion to an anti-glycophorin A antibody V region. Many such constructs activated EPO receptors in vitro and stimulated RBC and not platelet production in mice but nonetheless enhanced thrombosis in mice and caused cell adhesion between RBCs and EPO receptor-bearing cells. Based on a protein-structural model of the RBC surface, an anti-glycophorin/EPO fusion is rationally designed herein that does not induce adhesion in vitro or enhance thrombosis in vivo. Thus, meso-scale geometry can inform design of synthetic-biological systems.

### Introduction

Hormones and cytokines act on receptors on diverse cells to achieve coordinated biological responses. Some activities are desired while others constitute drug side effects. For example, cortisol acts on the glucocorticoid receptor in immune cells and is anti-inflammatory, but acts on other cells to increase blood sugar and induce bone resorption. Similarly, erythropoietin (EPO) stimulates red blood cell production but also increases thrombotic activity. These responses are adaptive in the context of hemorrhage, but increased thrombotic activity may underlie the increased frequency of heart attacks and strokes seen in EPO-treated kidney failure patients.

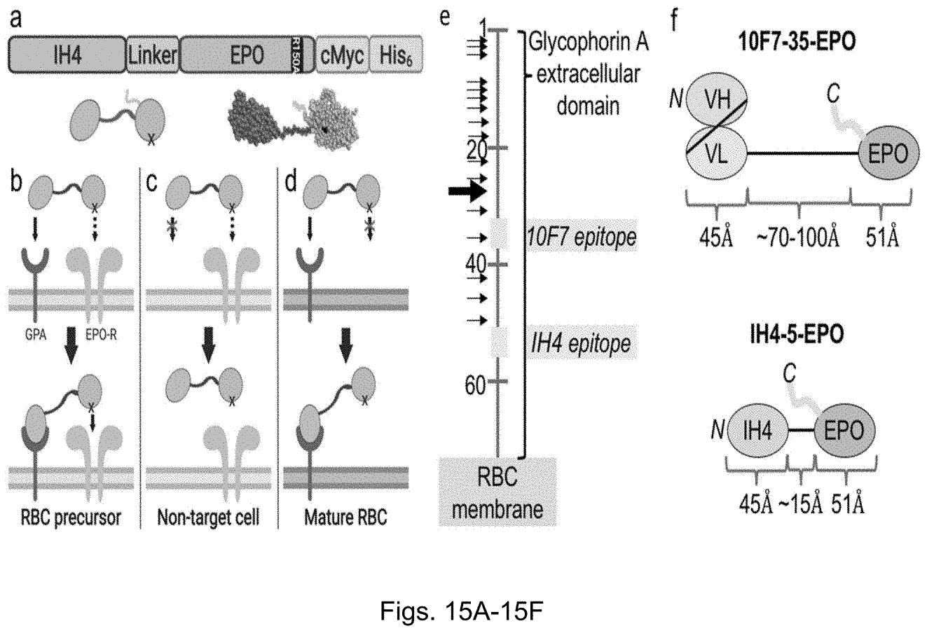

A class of AND-Gate proteins, “Chimeric Activators,” in which a cytokine or hormone is mutated to reduce its activity, and attached to a targeting element that binds to another protein on a subset of receptor-bearing cells is depicted in FIGS. 15A-15F. Chimeric activators are similar to other fusion proteins such as blinatumomab^{1,2} or immunocytokines,³ which connect one cell to another. Thus, implicit in this design is the potential to crosslink cells, whether desired or not. Understanding the quantitative and structural determinants for engineered cell-cell interaction is important in design of next-generation CAR-T cells, bispecific T-cell engagers, and other fusion proteins.⁴⁻⁶

An AND-gated EPO was constructed that activates EPO-Rs on RBC precursors but not on other cells.^{7,8} “Targeted EPO” proteins consist of an antibody element that binds to glycophorin A (GPA), EPO with a mutation (Arg150Ala) that reduces EPO activity, and a connecting linker (FIG. 15A).^{7,8} GPA is abundant on RBCs and late RBC precursors (800,000 and 50,000 copies per cell, respectively).^{7,9,10} GPA is not expressed on cells that control thrombosis, blood pressure, and tumor angiogenesis, or solid tumor cells. EPO receptors (EPO-Rs) on late RBC precursors mediate EPO-dependent maturation, but then disappear.^{7,11,12} Targeted EPO was therefore designed to bind to late RBC precursors and stimulate EPO-Rs on only those cells (FIG. 15B). On non-target cells, it fails to activate EPO-Rs because the mutated EPO alone binds poorly to receptors (FIG. 15C). Binding to GPA on mature RBCs is a desirable secondary consequence as it extends the plasma half-life of the fusion protein.⁷ However, EPO bound to mature RBCs could interact with EPO-Rs on endothelial cells¹³ or immune cells such as macrophages¹⁴ and mediate undesired cell adhesion or signaling (FIGS. 15D, 15E).⁷

Results and Discussion

The goal of the present work is to define structural and quantitative features of anti-GPA/EPO chimeric activators that act specifically in cis on GPA-expressing cells, to avoid side effects that may arise from unwanted cell-cell interaction. Previously a form of Targeted EPO consisting of V regions from the 10F7 antibody, a 35-amino acid linker and EPO with the weakening mutation R150A was characterized; this version is termed 10F7-35-EPO(R150A) (FIG. 15F). This protein specifically stimulated production of RBCs but not platelets, the latter being considered an indicator of for off-target cell effects. To directly assess the impact of this molecule on thrombosis, a ‘bleeding time’ assay was performed in which the tail of a treated mouse is transected and the time to cessation of bleeding is measured.

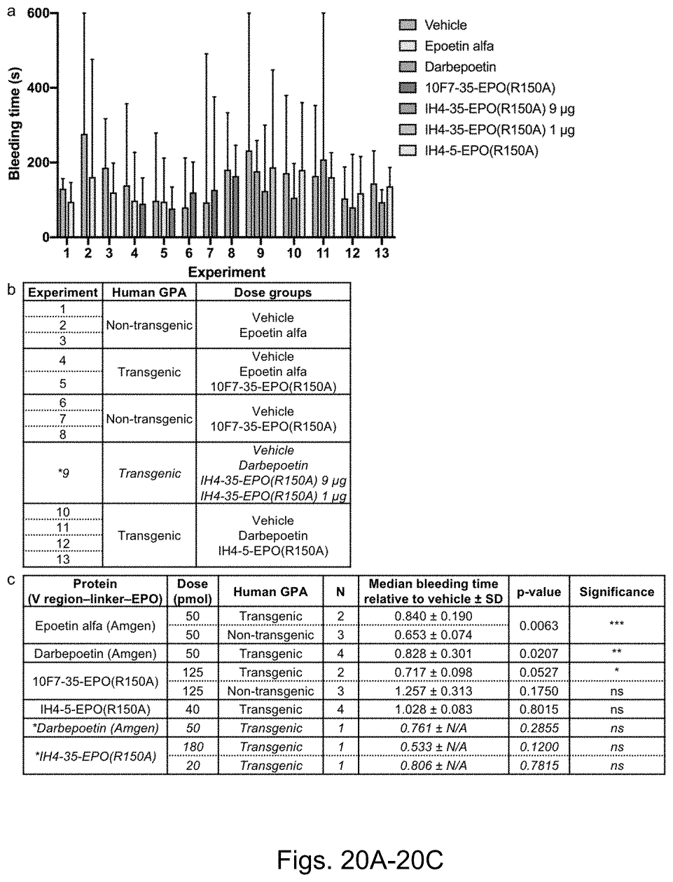

Contrary to expectation, the 10F7-35-EPO(R150A) form of Targeted EPO, while showing target cell-specific activity in vitro and in vivo, exhibited pro-thrombotic tendency in vivo in the bleeding time assay (FIGS. 20A-20C). It was hypothesized that there were two mechanisms by which this might occur. First, the antibody element could, by itself, promote blood clotting. Second, the RBC-bound Targeted EPO could crosslink RBCs with other cells bearing EPO-Rs, such as vascular endothelial cells or leukocytes, slowing blood flow. To determine whether any of these hypotheses was correct a series of Targeted EPO variants, was constructed, testing anti-GPA antibody elements (10F7, 1C3, R18 and IH4), fusion protein configurations, and linker lengths.

Targeted EPO Activates EPO-Rs in a GPA-Dependent Manner In Vitro and In Vivo

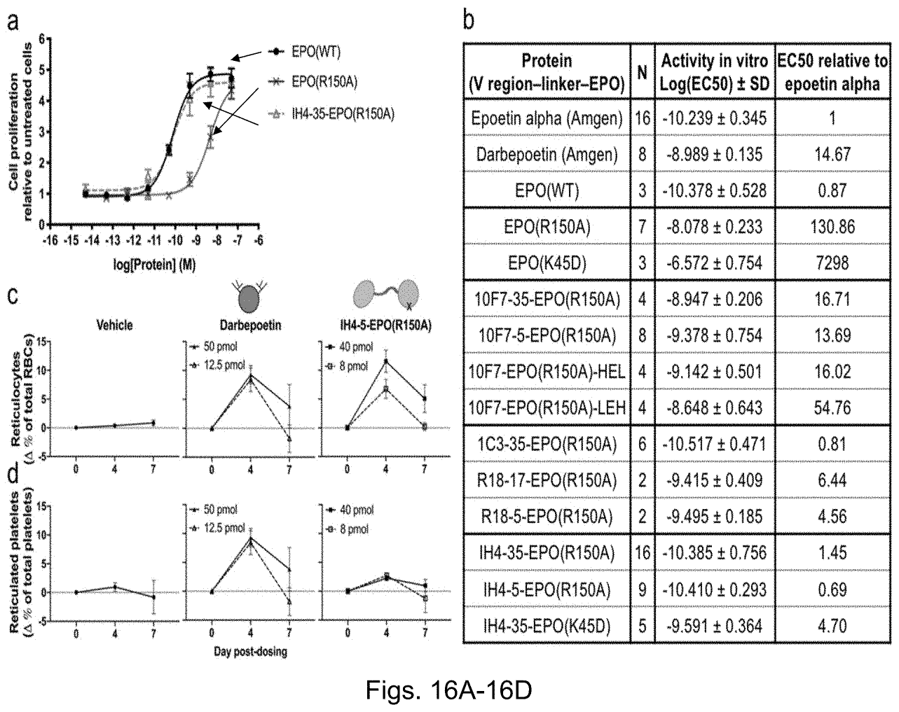

Diverse forms of Targeted EPO activate EPO-Rs in a GPA-dependent manner in vitro. TF-1 cells, which express both GPA and EPO-Rs, were treated with several forms of Targeted EPO, and their proliferation was measured 72 hr post-treatment (FIGS. 16A, 16B). Unfused EPO(R150A) showed about 100-fold reduced activity compared to EPO(WT). When EPO(R150A) was fused to an anti-GPA antibody element, its activity was rescued by 10-100-fold (FIGS. 16A, 16B). In particular, IH4-EPO(R150A) and 1C3-EPO(R150A) were most potent among other forms of Targeted EPO in which different GPA antibodies were used.

Three configurations of Targeted EPO were tested to address whether restricting the movement of EPO would affect EPO activity and potentially cause GPA-bound EPO to preferentially act in cis on targeted cells. The conventional scFv-EPO configuration is designed as antibody heavy chain—light chain—EPO (“HLE”). It was modified such that EPO would be between the heavy and light chains; this is feasible because the N- and C-termini of EPO are close in the folded structure. The resulting configurations (“HEL” and “LEH”) were expressed and tested. Both 10F7-EPO(R150A) and 1C3-EPO(R150A) stimulated proliferation of TF-1 cells largely independently of the configuration, indicating that orienting EPO in one way does not significantly constrain its receptor binding in cis (FIG. 16B and Table 4).

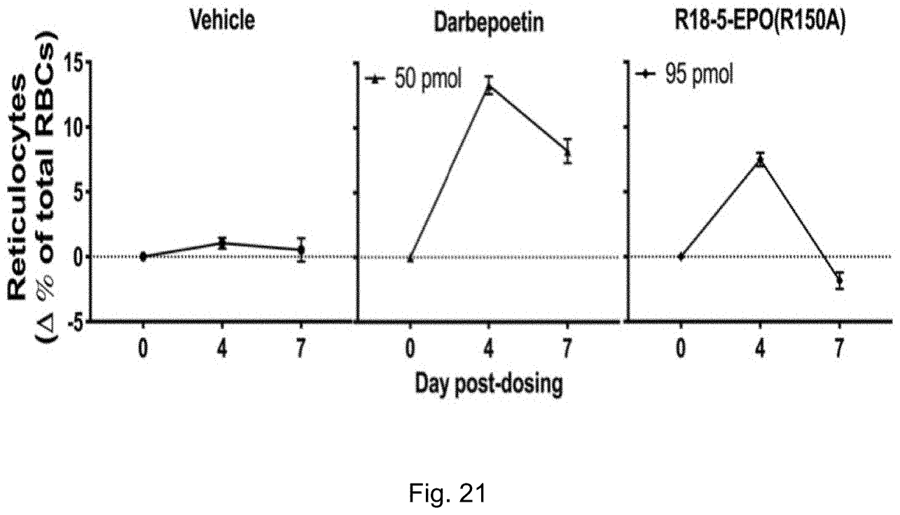

The GPA-mediated enhancement EPO(R150A) activity was independent of the linker length in the various fusion proteins T the linker length of 10F7-EPO(R150A), R18-EPO(R150A), R18-EPO(K45D), IH4-EPO(R150A), and IH4-EPO(K45D) were varied from 5 to 35 amino acids (about 10-80 Angstroms). The fusion proteins containing the same antibody and EPO elements but linkers of different lengths showed similar EC50s in cell-based assays (FIG. 16B and Table 4). Results with the rotationally constrained and short-linker constructs are consistent with the idea that GPA is an intrinsically disordered protein that is highly flexible, so that diverse fusion proteins bound to GPA can still allow binding of EPO with EPO-R. When tested in vivo, various forms Targeted EPO specifically stimulated production of RBCs but not platelets. In vivo target cell specificity and efficacy of several Targeted EPO proteins was assessed in mice that are transgenic for human GPA, following the experimental paradigm of Burrill et al.⁷ Darbepoetin, an extended half-life form of EPO, is a control to represent non-targeted EPO activity. Mice were injected ip with vehicle, darbepoetin, or a Targeted EPO, and their reticulocytes and reticulated platelets were measured to indicate activity on target cells and non-target cells, respectively. Mice treated with 2 mcg of darbepoetin induced comparable increases in both new RBCs and new platelets (FIG. 16C, 16D and FIG. 21).⁷ Mice treated with 2 meg of IH4-5-EPO(R150A) showed a similar increase in reticulocytes while the reticulated platelets were not significantly changed (FIG. 16C, 16D). IH4-5-EPO(R150A) was most potent in vivo of the forms of Targeted EPO tested: 10F7-35-EPO(R150A), and R18-5-EPO(R150A) stimulated the production of RBC less strongly than IH4-5-EPO(R150A), though all were significantly more potent than untargeted EPO(R150A) (FIG. 21).⁷

The Antibody Element IH4 does not Induce ‘RBC Inflammation’

The anti-GPA antibody element in forms of Targeted EPO might induce an inflammatory phenotype that could enhance blood clotting or cause other undesired side effects and also confound interpretation of experimental treatment with fusion proteins. Various anti-GPA antibodies and V region elements induce a constellation of effects on RBCs, including increased stiffness during shear stress, decreased membrane fluidity, secretion of ATP, production of reactive oxygen species, and phosphorylation of Tyr8 of Band 3. Divalent IgG-type antibodies against GPA induce at least some and likely all of these phenotypes, but only a subset of monovalent Fabs, scFvs and VHHs induce this RBC response in a binding epitope-dependent manner (FIG. 15E)¹⁵⁻²³.

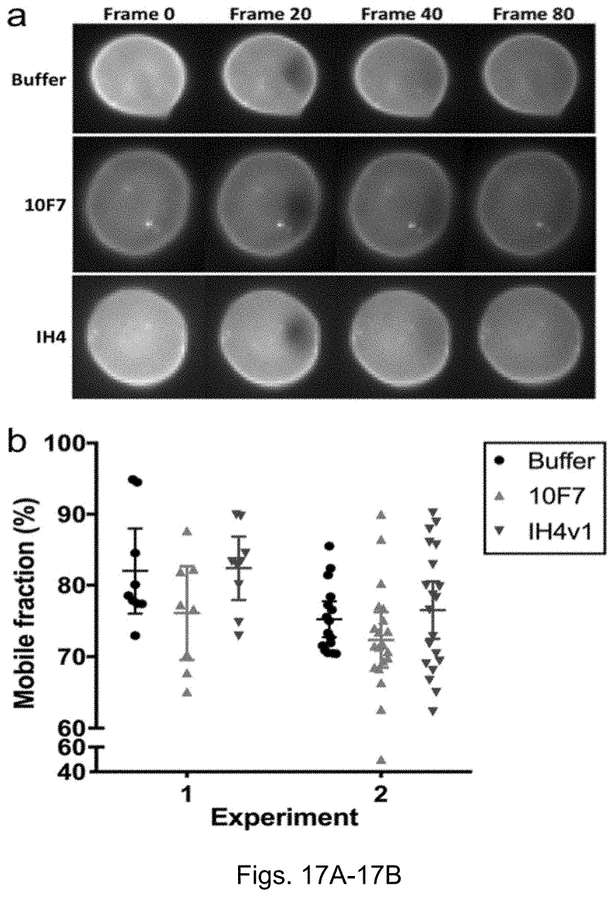

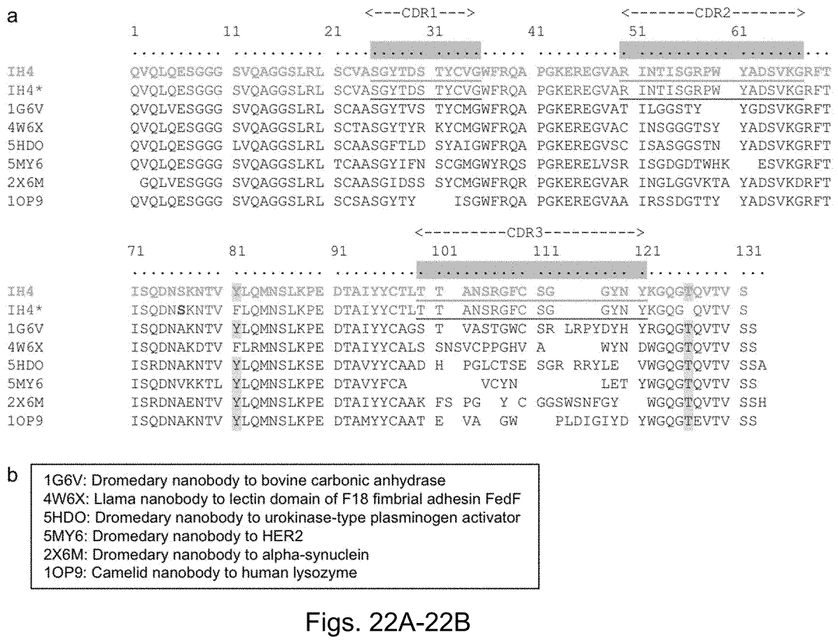

The single-chain camelid antibody (VHH or nanobody) element IH4 itself lacks stiffening/inflammatory activity when it is bound to GPA, and therefore is a good candidate targeting element.²⁰ This element binds to the membrane-proximal epitope (₅₂YPPE₅₅) on human GPA.^{21,24} We tested A variant of IH4 in which the framework regions 3 and 4 described in the U.S. Pat. No. 9,879,090^{24,25} were converted to the consensus sequence to improve expression was tested (FIGS. 22A-22B). It was confirmed by fluorescence recovery after photobleaching (FRAP) that 10F7 induced lower membrane lipid mobility in RBCs, while IH4 did not (FIGS. 17A, 17B). IH4 appears to bind most tightly of the known anti-GPA V region elements. Therefore, IH4 was chosen as a candidate antibody element for a Targeted EPO that would not induce undesired signaling and would also limit potential interaction of EPO with EPO-Rs on other cells.

Geometry of GPA and Targeted EPO Determines Potential for Cell Crosslinking

In the design of Targeted EPO, the intention is that the antibody element and EPO bind in cis to receptors on the same cell surface (FIG. 15B). However, fusion proteins comprising two binding elements have the potential to crosslink different cells that each bears a receptor for a component of the fusion protein (FIG. 15D). To address this, the accessibility of antibodies to the EPO element of Targeted EPO variants bound to RBCs, and the ability of different Targeted EPOs to promote binding between RBCs and tumor cells expressing EPO-Rs were tested.

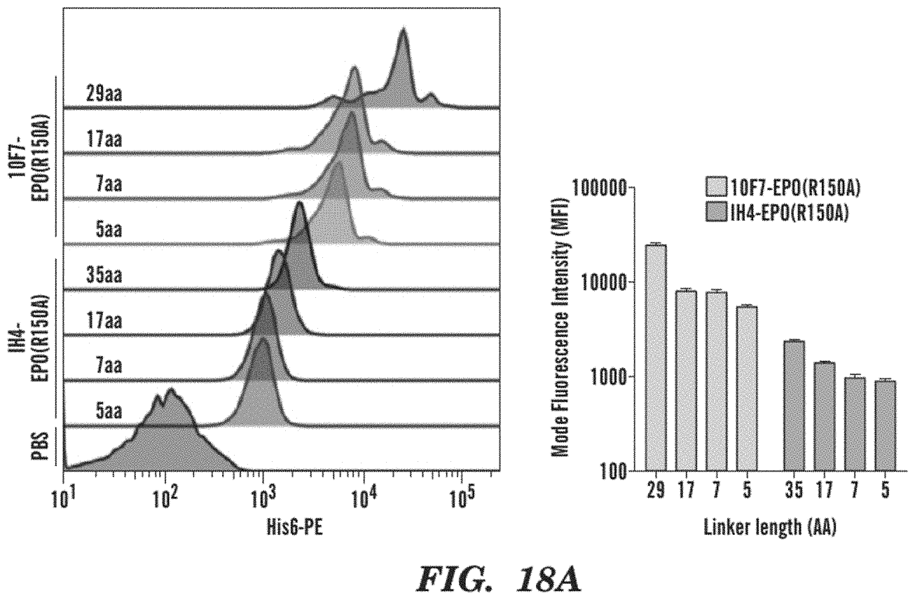

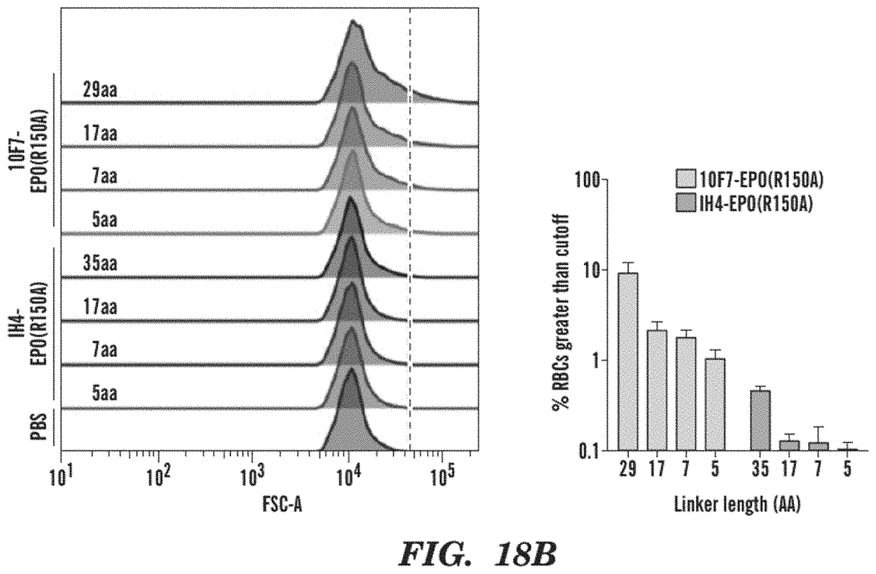

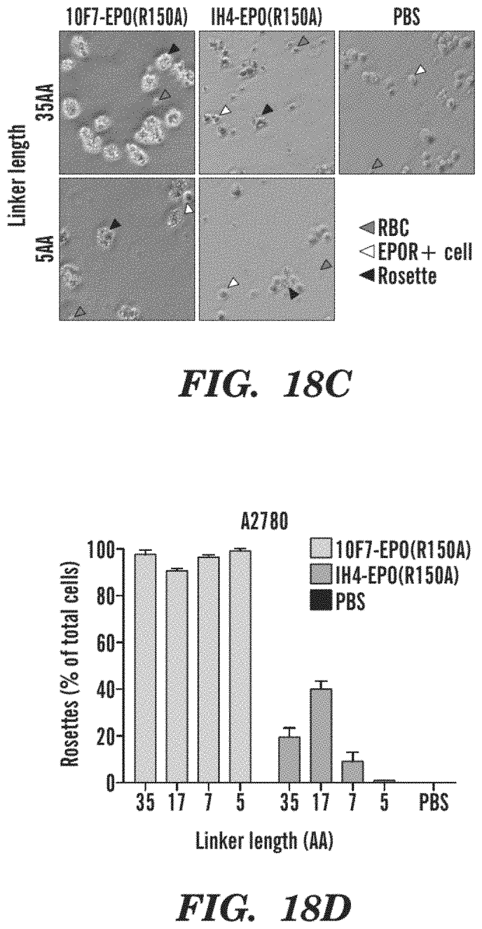

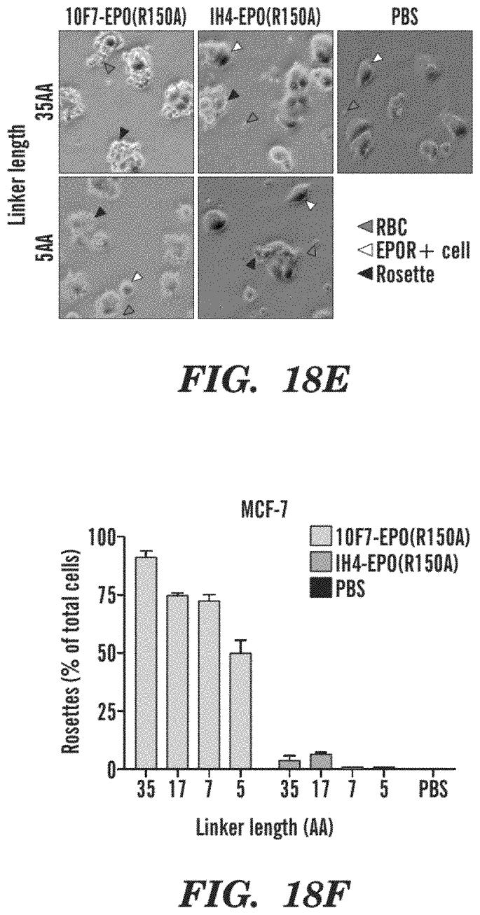

When different forms of Targeted EPO are bound to RBCs via GPA, the antibody accessibility of the EPO element decreases when the linker length is shortened and the epitope on GPA is closer to the cell membrane (FIG. 18A, 18B). A series of proteins in which EPO was fused to either IH4 or 10F7 and the linker length was systematically varied were compared. The proteins had a His₆ tag at the C-terminus. RBCs were incubated with the fusion protein, and then the accessibility of EPO in the bound state was assessed via flow cytometry by binding of a PE-conjugated anti-His₆ antibody. For both IH4 and 10F7 fusion proteins, PE signals progressively decreased as the linker was shortened, indicating that the fusion proteins with shorter linkers were better masked from interacting with the antibody (FIG. 18A). In addition, crosslinking of RBCs by the divalent anti-His₆ antibody increased with the linker length, as seen in forward scattering signals evidenced by a shoulder to the right of the main peak (FIG. 18B). Also, regardless of the linker length, all IH4 fusion proteins had lower accessibility and fewer RBC crosslinking events by anti-His₆ antibody than corresponding 10F7 fusion proteins, indicating that the binding epitope of the antibody element also affects the accessibility of EPO (FIG. 18A, 18B).

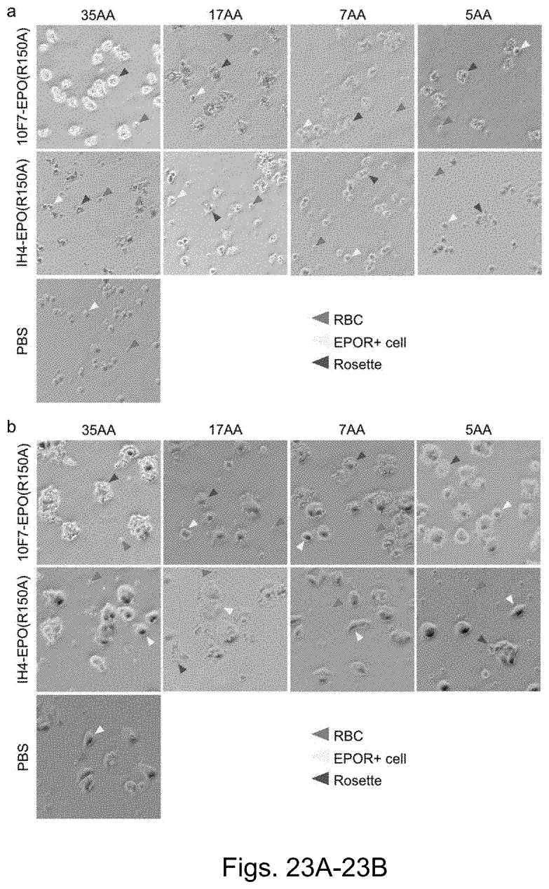

Forms of Targeted EPO with long linkers and a membrane-distal epitope on GPA mediate adhesion in vitro between RBCs and cells expressing EPO-Rs. A rosette assay was used for cell-cell interaction to address whether the anti-GPA/EPO fusion proteins might promote adherence of RBCs to cells from the tumor lines A2780 and MCF-7, which express EPO-R.²⁶ All forms of Targeted EPO containing the 10F7 scFv (epitope at about ₃₄YAATP₃₈) showed a high level of rosette formation, with most or all of the tumor cells binding to 3 or more RBCs. In contrast, the frequency of rosette formation with Targeted EPOs containing the IH4 nanobody (epitope: ₅₂YPPE₅₅) was much lower. Targeted EPO forms with shorter linkers generally induced less rosette formation. The effect of epitope position and linker length were synergistic: the IH4-EPO(R150A) with a 5 amino acid linker showed essentially no rosette formation by either A2780 or MCF-7 cells (FIGS. 18C-18F and FIGS. 23A-23B).

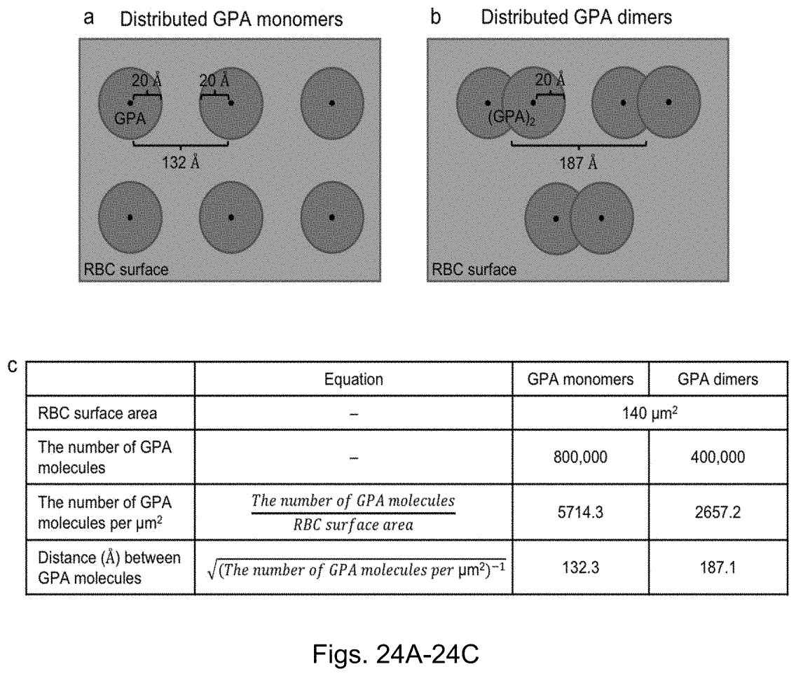

The results of the antibody accessibility and rosette formation experiments are consistent with a geometric model of the RBC surface in which GPA extends from the cell and shields the cell from interactions with other cells. GPA is densely packed on the RBC surface, with about 800,000 monomers in a surface area of 140 square microns,²⁷ such that the average distance between GPA molecules is about 130-190 Angstroms (FIGS. 24A-24C). The membrane-distal N-terminal ˜45 amino acids of GPA include 17 sites for O- and N-glycosylation, with oligosaccharides that extend at least about 20 Angstroms from the peptide backbone (for the 0-type blood group antigen). It is likely that the N-terminal 45 amino acids of GPA are largely unstructured and would be about 90-130 Angstroms long in an extended conformation.²⁸ By comparison, in an IgG antibody in the T conformation the tips of the V regions are about 150 Angstroms apart, and in an scFv-(SGGGS)-EPO fusion protein, the components would be 45, 15 and 51 Angstroms end-to-end. These considerations suggest that antibody access to membrane-proximal epitopes could be limited, and that contact between a GPA-bound IH4-(SGGGS)-EPO molecule and EPO-Rs on another cell would be sterically very difficult (FIG. 15E, 15F).

IH4-5-EPO(R150A) does not Shorten Tail Vein Bleeding Time in Mice

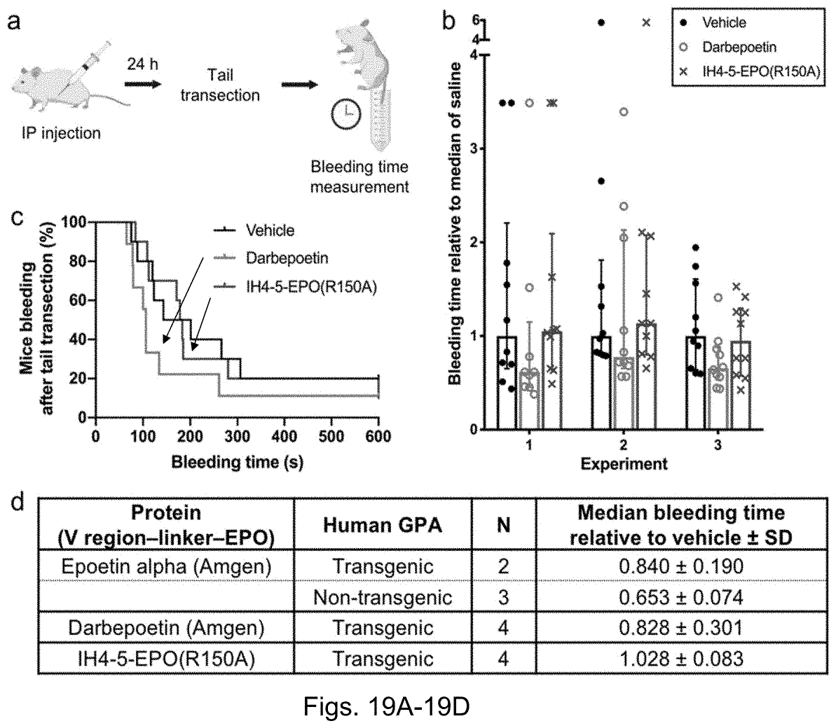

Since IH4-5-EPO(R150A) is non-inflammatory and shielded from promoting cell-cell interactions, the systemic effects of IH4-5-EPO(R150A) on thrombosis in mice were tested for. HuGPA-transgenic mice were injected ip with saline, darbepoetin, 10F7-35-EPO(R150A) or IH4-5-EPO(R150A). One day after injection, tail vein bleeding time was measured (FIG. 19A). Darbepoetin, 10F7-35-EPO(R150A) and IH4-5-EPO(R150A) were injected at a dose that resulted in similar effect in mice, as measured by reticulocyte counts. In mice treated with darbepoetin or 10F7-35-EPO(R150A), the bleeding time was shortened by about 30%, whereas mice treated with IH4-5-EPO(R150A) showed no change in bleeding time (FIGS. 19B-19D). These results indicate that IH4-5-EPO(R150A) does not enhance thrombotic side effects, in contrast to the non-targeted form EPO that acts on EPO-Rs on multiple cell types.

When the data from these experiments were normalized to the vehicle controls of the same experiments and then combined, the difference between vehicle and darbepoetin (p=0.0098) or IH4-5-EPO(R150A) and darbepoetin (p=0.017) was significant. When data were compared across all experiments with dose groups including 10F7-35-EPO(R150A) and epoetin alfa, these molecules also showed a statistically significant decrease in bleeding time relative to the vehicle control (FIGS. 20A-20C). These results are consistent with previous studies indicating that non-targeted EPO proteins reduce bleeding time in rodents.²²

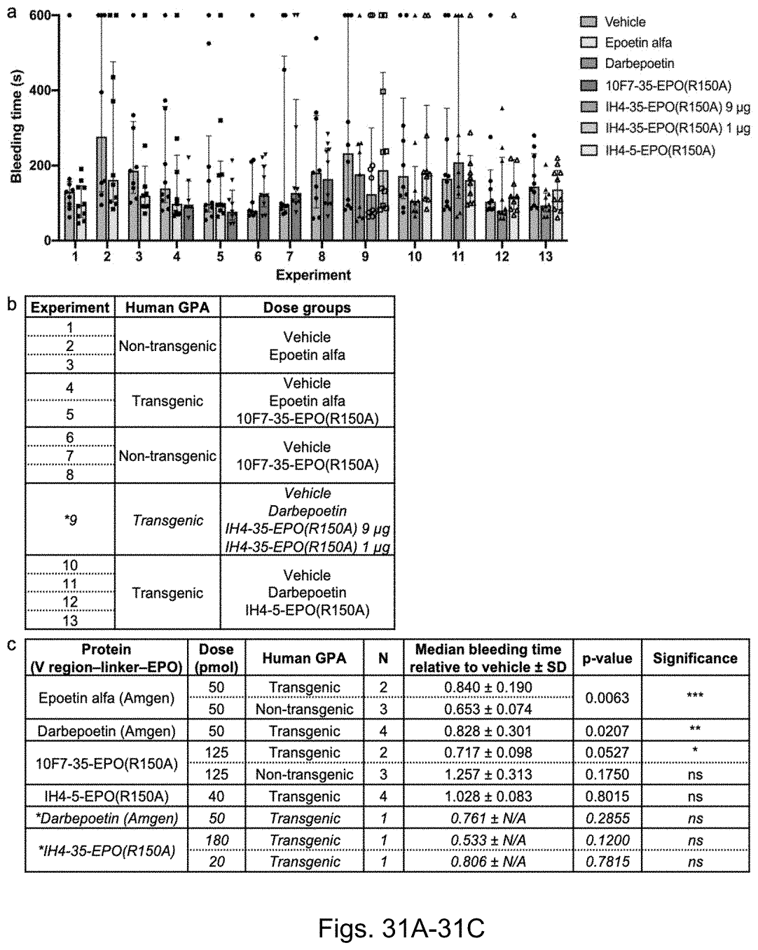

Interestingly, a form of Targeted EPO with the non-inflammatory IH4 and a long linker, IH4-35-EPO(R150A), shortened bleeding time by 30%, similarly to darbepoetin (FIGS. 31A-31C) This is consistent with the hypothesis that a Targeted EPO containing a long linker can enhance thrombosis by crosslinking RBCs and different cells in the bloodstream.

Discussion

The design of a fusion protein must consider questions about quantitative properties of each individual component and spatial orientation of interacting proteins to achieve high efficacy while minimizing unwanted interactions. Such considerations become particularly important in AND-Gate bifunctional protein systems in which an activity element interacts with a receptor that is abundant on different cell types. EPO-Rs are found on numerous cell types besides RBC precursors, including platelet precursors, vascular endothelial cells, liver cells, and white blood cells such as macrophages.

In this work, this problem was addressed for “Targeted EPO” molecules, fusion proteins that bind to GPA and weakly to EPO-R and are thus AND-gated to bind to only cells with both of these transmembrane proteins—namely late RBC precursors. The design goal of these proteins is to specifically activate EPO-R only on late RBC precursors as a result of initial binding to GPA, followed by binding in cis to EPO receptor due to the high local concentration of EPO (FIG. 15B). We found that:
1. Some Targeted EPO proteins can mediate adhesion between mature RBCs that express only GPA and other cells that express only EPO-R.
2. The position of the epitope on GPA and the length of the linker between the GPA binding element and the EPO element can determine whether a given Targeted EPO can mediate cell adhesion.
3. The cell adhesion results can be interpreted with a geometric model in which GPA extends from the RBC surface and creates a limit on how close another cell can approach. To mediate cell adhesion, the EPO element must be able to extend beyond the membrane-distal end of GPA.
4. More broadly, cell-cell interaction may be regulated by abundant cell surface proteins that may sterically prevent close approach of the cells. This needs to be considered when constructing engineered cell interaction systems, such as CAR-T cells and crosslinking bispecfic antibodies. In addition, natural cell interactions may be regulated by such phenomena.

A major challenge for synthetic biology is that artificial biological systems have the potential for undesired interactions that can lead to system failure. These experiments illustrate geometric principles that natural systems use in achieving specificity.

Methods

Methods for cell culture, DNA construction, protein expression and purification, in vitro measurement of erythropoietin activity by cell proliferation assays, and measurement of mouse reticulocytes and reticulated platelets were the same as described in Burrill et al.⁴ Fluorescence recovery after photobleaching (FRAP) was performed as described by Khoory et al.¹⁷

EPO accessibility and RBC crosslinking measurement by flow cytometry. RBCs (Zen-Bio, Research Triangle Park, N.C.; 0.2% v/v in PBS-glucose) were incubated with fusion proteins for 1 hr at 4° C., washed once with PBS-glucose (PBS, 5 mM glucose, pH 7.4), incubated with anti-His₆-PE antibody (Abcam) for 30 min at 4° C., and washed once with PBS-glucose. Fusion protein concentration was calculated based on the K_{D} of the monovalent 10F7 and IH4 antibody elements (95 nM and 33 nM, respectively) to achieve 80% saturation of GPA. The cells were added to a 96-well U-bottom plate and analyzed by flow cytometry using standard techniques.

RBC rosette assay. A2780 and MCF-7 cells were plated on a 6-well plate containing 12×12 mm coverslips and incubated overnight at 37° C. Next day, a mixture of RBCs (0.2% v/v) and fusion proteins was prepared in RPMI-1640 without bicarbonate and incubated for 30 min at 23° C. Coverslips with cells were washed twice with PBS and transferred to a new 6-well plate. The RBC-fusion protein mixture was added on top of each coverslip and incubated for 1 hr at 37° C. Coverslips were gently washed twice with PBS. Rosettes were imaged on a Nikon Eclipse TE300 inverted phase-contrast microscope. Rosettes were defined as adherent cells bound by 3 or more RBCs and were shown as a percentage of at least 160 cells from at least 8 fields.

Mouse tail vein bleeding time measurement. Experiments were performed in a blinded manner. Ten mice per dose group received a single ip injection with saline or test protein. Next day, mice were anaesthetized. The tail was transected 3 mm from the tip, and the body of the mouse was placed on a heated pad over a 37° C. water bath containing tubes with 50 mL of saline, such that 0.5-2 mm of the transected tail was placed in the saline within 2 seconds of transection. The bleeding time was recorded when bleeding stopped for one minute; if bleeding re-started within one minute the initial stopping time was not counted. Bleeding time longer than 10 minutes were recorded as 10 minutes.

### REFERENCES

1. Buie, L. W.; Pecoraro, J. J.; Horvat, T. Z.; Daley, R. J., Blinatumomab: A First-in-Class Bispecific T-Cell Engager for Precursor B-Cell Acute Lymphoblastic Leukemia. Ann Pharmacother 2015, 49 (9), 1057-67.
2. Goebeler, M. E.; Bargou, R., Blinatumomab: a CD19/CD3 bispecific T cell engager (BiTE) with unique anti-tumor efficacy. Leuk Lymphoma 2016, 57 (5), 1021-32.
3. Sondel, P. M.; Gillies, S. D., Current and Potential Uses of Immunocytokines as Cancer Immunotherapy. Antibodies (Basel) 2012, 1 (2), 149-171.
4. Zah, E.; Lin, M. Y.; Silva-Benedict, A.; Jensen, M. C.; Chen, Y. Y., T Cells Expressing CD19/CD20 Bispecific Chimeric Antigen Receptors Prevent Antigen Escape by Malignant B Cells. Cancer Immunol Res 2016, 4 (6), 498-508.
5. Chang, Z. L.; Chen, Y. Y., CARs: Synthetic Immunoreceptors for Cancer Therapy and Beyond. Trends Mol Med 2017, 23 (5), 430-450.
6. Baeuerle, P. A.; Reinhardt, C., Bispecific T-cell engaging antibodies for cancer therapy. Cancer Res 2009, 69 (12), 4941-4.
7. Burrill, D. R.; Vernet, A.; Collins, J. J.; Silver, P. A.; Way, J. C., Targeted erythropoietin selectively stimulates red blood cell expansion in vivo. Proc Natl Acad Sci USA 2016, 113 (19), 5245-50.
8. Taylor, N. D.; Way, J. C.; Silver, P. A.; Cironi, P., Anti-glycophorin single-chain Fv fusion to low-affinity mutant erythropoietin improves red blood cell-lineage specificity. Protein Eng Des Sel 2010, 23 (4), 251-60.
9. Merry, A. H.; Thomson, E. E.; Anstee, D. J.; Stratton, F., The quantification of erythrocyte antigen sites with monoclonal antibodies. Immunology 1984, 51, 793-800.
10. Chen, K.; Liu, J.; Heck, S.; Chasis, J. A.; An, X.; Mohandas, N., Resolving the distinct stages in erythroid differentiation based on dynamic changes in membrane protein expression during erythropoiesis. Proc Natl Acad Sci USA 2009, 106 (41), 17413-17418.
11. Wickrema, A.; Crispino, J. D., Erythroid and megakaryocytic transformation. Oncogene 2007, 26 (47), 6803-15.
12. Elliott, S.; Sinclair, A.; Collins, H.; Rice, L.; Jelkmann, W., Progress in detecting cell-surface protein receptors: the erythropoietin receptor example. Ann Hematol 2014, 93 (2), 181-92.
13. Anagnostou, A.; Lee, E. S.; Kessimian, N.; Levinson, R.; Steiner, M., Erythropoietin has a mitogenic and positive chemotactic effect on endothelial cells. Proc Natl Acad Sci USA 1990, 87, 5978-5982.
14. Lifshitz, L.; Tabak, G.; Gassmann, M.; Mittelman, M.; Neumann, D., Macrophages as novel target cells for erythropoietin. Haematologica 2010, 95 (11), 1823-31.
15. Chasis, J. A.; Mohandas, N.; Shohet, S. B., Erythrocyte membrane rigidity induced by glycophorin A-ligand interaction. Evidence for a ligand-induced association between glycophorin A and skeletal proteins. J Clin Invest 1985, 75 (6), 1919-26.
16. Chasis, J. A.; Reid, M. E.; Jensen, R. H.; Mohandas, N., Signal transduction by glycophorin A: role of extracellular and cytoplasmic domains in a modulatable process. The Journal of Cell Biology 1988, 107, 1351-1357.
17. Chasis, J. A.; Mohandas, N., Red blood cell glycophorins. Blood 1992, 80 (8), 1869-1879.
18. Knowles, D. W.; Chasis, J. A.; Evans, E. A.; Mohandas, N., Cooperative action between band 3 & glycophorin A in human erythrocytes: immobilization of band 3 induced by antibodies to glycophorin A. Biophysical Journal 1994, 66, 1726-1732.
19. Paulitschke, M.; Nash, G. B.; Anstee, D. J.; Tanner, M. J. A.; Gratzer, W. B., Perturbation of red blood cell membrane rigidity by extracellular ligands. Blood 1995, 86 (1), 342-348.
20. Giger, K.; Habib, I.; Ritchie, K.; Low, P. S., Diffusion of glycophorin A in human erythrocytes. Biochim Biophys Acta 2016, 1858 (11), 2839-2845.
21. Habib, I.; Smolarek, D.; Hattab, C.; Grodecka, M.; Hassanzadeh-Ghassabeh, G.; Muyldermans, S.; Sagan, S.; Gutierrez, C.; Laperche, S.; Le-Van-Kim, C.; Aronovicz, Y. C.; Wasniowska, K.; Gangnard, S.; Bertrand, O., V(H)H (nanobody) directed against human glycophorin A: a tool for autologous red cell agglutination assays. Anal Biochem 2013, 438 (1), 82-9.
22. Che, A.; Cherry, R. J., Loss of rotational mobility of band 3 proteins in human erythrocyte membranes induced by antibodies to glycophorin A. Biophysical Journal 1995, 68, 1881-1887.
23. Gardner, B.; Parsons, S. F.; Merry, A. H.; Anstee, D. J., Epitopes on sialoglycoprotein a: evidence for heterogeneity in the molecule. Immunology 1989, 68, 283-289.
24. Bertrand, O.; Habib, I.; Smolarek, D. Fusion proteins and Immunoconjugates and uses thereof. WO 2014/135528 A1, 2014.
25. Bertrand, O.; Habib, I.; Kremlin-Bicetre, L.; Smolarek, D. Fusion proteins and immunoconjugates and uses thereof which are specific for glycophorin A. 2018.
26. Pradeep, S.; Huang, J.; Mora, E. M.; Nick, A. M.; Cho, M. S.; Wu, S. Y.; Noh, K.; Pecot, C. V.; Rupaimoole, R.; Stein, M. A.; Brock, S.; Wen, Y.; Xiong, C.; Gharpure, K.; Hansen, J. M.; Nagaraja, A. S.; Previs, R. A.; Vivas-Mejia, P.; Han, H. D.; Hu, W.; Mangala, L. S.; Zand, B.; Stagg, L. J.; Ladbury, J. E.; Ozpolat, B.; Alpay, S. N.; Nishimura, M.; Stone, R. L.; Matsuo, K.; Armaiz-Pena, G. N.; Dalton, H. J.; Danes, C.; Goodman, B.; Rodriguez-Aguayo, C.; Kruger, C.; Schneider, A.; Haghpeykar, S.; Jaladurgam, P.; Hung, M. C.; Coleman, R. L.; Liu, J.; Li, C.; Urbauer, D.; Lopez-Berestein, G.; Jackson, D. B.; Sood, A. K., Erythropoietin Stimulates Tumor Growth via EphB4. Cancer Cell 2015, 28 (5), 610-622.
27. Ballas, S. K., Erythrocyte concentration and volume are inversely related. Clinica Chimica Acta 1987, 164, 243-244.
28. Welsh, E. J.; Thom, D.; Morris, E. R.; Rees, D. A., Molecular organization of glycophorin A: implications for membrane interactions. Biopolymers 1985, 24, 2301-2332.
29. Khoory, J.; Estanislau, J.; Elkhal, A.; Lazaar, A.; Melhom, M. I.; Brodsky, A.; Illigens, B.; Hamachi, I.; Kurishita, Y.; Ivanov, A. R.; Shevkoplyas, S.; Shapiro, N. I.; Ghiran, I. C., Ligation of Glycophorin A Generates Reactive Oxygen Species Leading to Decreased Red Blood Cell Function. PLoS One 2016, 11 (1), e0141206.
30. Melhorn, M. I.; Brodsky, A. S.; Estanislau, J.; Khoory, J. A.; Illigens, B.; Hamachi, I.; Kurishita, Y.; Fraser, A. D.; Nicholson-Weller, A.; Dolmatova, E.; Duffy, H. S.; Ghiran, I. C., CR1-mediated ATP release by human red blood cells promotes CR1 clustering and modulates the immune transfer process. J Biol Chem 2013, 288 (43), 31139-53.

### Example 12: Supplemental Information for Example 11

Cell culture. FreeStyle 293-F, FreeStyle CHO-S, and CHO DG44 cells were obtained from Invitrogen (Carlsbad, Calif.), and were cultured in FreeStyle 293 Expression Medium, complete FreeStyle CHO Expression Medium, and complete CD DG44 (Invitrogen), respectively. Human erythroleukemia TF-1, human ovarian cancer A2780, and human breast cancer MCF-7 were obtained by ATCC (Manassas, Va.). TF-1 was cultured in RPMI-1640 with 10% FBS, 100 U/mL penicillin, 100 U/mL streptomycin, and 2 ng/mL recombinant human granulocyte macrophage colony-stimulating factor (GM-CSF; PeproTech) unless specified otherwise. A2780 was cultured in RPMI-1640 with 10% FBS. MCF-7 was cultured in DMEM with 10% FBS and 0.01 mg/mL recombinant human insulin (PeproTech). 293-F, CHO-S, and CHO DG44 were cultured at 37° C. in 8% CO₂ with shaking at 2.35×g. TF-1, A2780, and MCF-7 were cultured at 37° C. in 5% CO₂.

DNA constructs. The DNA sequences for 10F7 and EPO (wildtype and mutants) were from GenBank (accession no. KX026660-3). The DNA sequences for IH4, R18, and 1C3 were derived by reverse translating and codon optimizing (Integrated DNA Technologies) the protein sequences adapted from the U.S. Pat. Nos. 9,879,090, 8,900,592, and patent application WO1993024630, respectively.¹⁻³ The IH4 sequence was modified to include a point mutation (Phe80Tyr) and an additional amino acid (Thr118) in the framework regions 3 and 4, respectively, as the reported sequence of IH4 may have had fortuitous changes from the germline sequence or typographical errors. The IH4 antibody with the original sequence in the patent was denoted as IH4*. The DNA sequences for glycine-serine linkers of various lengths were codon-optimized for expression in mammalian cells.

Protein expression and purification. Transient expression was performed in 293-F and CHO-S cells using pSecTag2A or pOptiVEC plasmids, and stable expression was performed using CHO DG44 cells using pOptiVEC plasmids according to the supplier's protocol. Protein expression was assayed by Western blotting cell supernatant using anti-His₆-HRP antibody (Abcam). Stably transfected cells were selected by hypoxanthine/thymidine (HT)-deficient CD OptiCHO™ medium (Invitrogen), and were subjected to one round of methotrexate (MTX; Sigma-Aldrich) genomic amplification as described before.⁴ Proteins from transient transfection or stable pools were purified as follows. Supernatant was concentrated to 5-8 mL using a 10 kDa cut-off Macrosep™ Advance centrifugal device (Pall). Concentrated protein was bound to 0.5-1 mL of His60 nickel or HisTalon™ cobalt resin (Takara Bio) for 0.5 h at 4° C. while rotating in a 10-mL Pierce disposable column (Thermo Scientific), and was washed and eluted using His60 or HisTalon™ Buffer Set (Takara Bio) according to the supplier's protocol. Cell supernatant and each purification fraction were analyzed by SDS-PAGE followed by Coomassie Blue staining. Eluted proteins were combined, desalted into endotoxin-free PBS (Teknova: 137 mM NaCl, 1.4 mM KH₂PO₄, 4.3 mM Na₂HPO₄, and 2.7 mM KCl, pH 7.4) using Econo-Pac 10DG columns (Bio-Rad), and concentrated to <1 mL using Macrosep™ Advance centrifugal device. For in vivo experiments, contaminating proteins were further removed by size exclusion chromatography (SEC) on Superdex 200™ 10/300 GL columns (GE Healthcare) using AKTA FPLC system (GE Healthcare) and endotoxin-free PBS as the running buffer. Desired protein fractions were combined and concentrated to <1 mL using Macrosep™ Advance centrifugal device. Proteins were stored at 4° C. throughout the described process, ultimately stored as aliquots at −80° C., and thawed once before use. Only endotoxin-free reagents were used.

Cell proliferation assays. TF-1 cells were seeded in a 96-well plate at 9.0×10³ cells per well in 90 μL of RPMI-1640 with serum and antibiotics (no GM-CSF). The purified proteins were serially diluted by 10-fold (10⁻⁷ to 10⁻¹⁴ M) and added to the cells. After 72 hours, cell proliferation was measured by CellTiter 96® AQᵤₑₒᵤₛ One Solution Cell Proliferation Assay (Promega). Absorbance at 490 nm was read on a BioTek Synergy Neo HTS microplate reader. Reported data represent mean±SEM of three replicates.

Flow cytometry for reticulocyte and reticulated platelet counts. HuGPA transgenic FVB mice were as described.⁵ This strain underwent embryo re-derivation at Charles River Laboratories. The homozygous huGPA transgene is embryonic-lethal but heterozygotes are phenotypically normal, so a breeding colony was maintained with screening for huGPA at each generation. Transgene expression was measured as described before.⁴

In experiments that measure efficacy of fusion and control proteins, five mice per dose group received a single intraperitoneal (ip) injection with saline, darbepoetin, or Targeted EPO in a 200 μL volume (diluted in saline or PBS) on Day 0. 1-5 μL of whole blood was collected by tail-nick in EDTA-coated tubes on days 0, 4, and 7 post-injection. Blood was analyzed immediately after collection by flow cytometry as described before.⁴ Thiazole orange (Sigma-Aldrich) was used to stain residual RNA in reticulocytes and reticulated platelets, and anti-CD41-PE antibody (BD Pharmingen) was used to stain total platelets. A stock solution (1 mg/mL) of thiazole orange was prepared in 100% methanol and was diluted 1:5,000 in PBS to make a 2× working solution. Anti-CD41-PE antibody was diluted 1:500 in either the 2× working solution of thiazole orange for stained samples or PBS for gating thiazole orange-negative population. 2 μL of whole blood was diluted 1:1,000 in 2 mL of PBS. Equal volumes (100 μL) of 2× working solution of anti-CD41-PE antibody with or without thiazole orange and diluted whole blood were mixed in a 96-well U-bottom plate and incubated for 30 min in the dark at RT. The fluorescence was measured on a LSRFortessa SORP flow cytometer equipped with an optional HTS sampler (BD Biosciences) using the following filter configuration: PE excitation, 561/50 mW; emission filter, BP 582/15; YFP excitation, 488/100 mW; emission filter, BP 540/25.

Preparation of human red blood cells. For FRAP, blood was obtained from healthy adult volunteers in accordance with the guidelines of, and approved by the Institutional Review Board of Beth Israel Deaconess Medical Center. Human blood was collected into HBSS++ containing 0.05% IgG-free BSA by venipuncture. RBCs were centrifuged at 5,000×g at 23° C. for 1 min, and were washed once with the same buffer. For flow cytometry and rosetting assays, human whole blood collected in an ACD tube by venipuncture was purchased from Zen-Bio (Research Triangle Park, N.C.). RBCs were isolated by centrifugation at 5,000×g at 23° C. for 1 min, and washed twice with PBS-glucose (PBS, 5 mM glucose, pH 7.4).

Fluorescence recovery after photobleaching (FRAP). RBCs (10% v/v in HBSS++ containing 0.1% IgG-free BSA) were incubated with lipid dye DiO (15 μg/mL) for 30 min at 23° C. in dark and washed once with HBSS++ containing 0.1% IgG-free BSA. Then RBCs were treated with GPA antibodies at 37° C. for 15 min. The concentration of antibody was determined by its K_{D} to achieve 80% saturation of GPA. RBCs were imaged using a 60× objective on an Olympus BX62 fluorescence microscope, bleached with Vector Controller Laser, and analyzed as described before.^{6,7} Reported data represent mean±95% confidence interval of 8-9 cells (experiment 1) or 16-20 cells (experiment 2). Data from each experiment were normalized to the buffer control in the same experiment to account for day-to-day variability, and the normalized values were combined across experiments. Mann-Whitney test was performed to determine p-values for individual and combined data sets.

Flow cytometry for EPO accessibility and RBC crosslinking measurement. The fluorescence was measured on a LSRFortessa SORP flow cytometer equipped with an HTS sampler (BD Biosciences) using the following filter configuration: PE excitation, 561/50 mW; emission filter, BP 582/15. Data were analyzed using FlowJo™ Data Analysis software (TreeStar, Ashland, Oreg.). Reported data represent mean±SEM of three replicates.

RBC rosette assay. A2780 and MCF-7 cells were plated at 2.0×10⁵ cells/mL in a 2 mL volume on a 12×12 mm coverslip (Electron microscopy sciences) in a well of a 6-well plate (Corning) and incubated overnight at 37° C. in 5% CO₂. The following day, a mixture of RBCs (0.2% v/v) and fusion proteins was prepared in binding medium (RPMI-1640 without bicarbonate) and incubated for 30 min at 23° C. Coverslips coated with cells were washed twice with PBS and transferred to a new 6-well plate. The RBC-fusion protein mixture (200 μL) was added on top of each coverslip and incubated for 1 hr at 37° C. Coverslips were gently washed twice with PBS. Rosettes were imaged on a Nikon Eclipse TE300 inverted phase-contrast microscope (Nikon, Melville, N.Y.), using a Retiga EXi CCD camera (QImaging, Surrey, Canada) controlled by iVision 4.7 software (BioVision). Rosettes were defined as adherent cells bound by 3 or more RBCs and were shown as a percentage of at least 160 cells from at least 8 fields. Reported data represent mean±SEM of at least two replicates.

Mouse tail vein bleeding time measurement. The tail transection assay of bleeding time was generally performed on mice as follows. On day 0, mice were injected with a test protein (or vehicle). When testing an unknown protein, it is important to also include mice that are injected with saline or PBS vehicle as a negative control, and EPO or darbepoetin as a positive control. Typically 10 mice per dose group were used. The experiments were performed in a blind manner: one experimenter performed the injections of proteins into the mice, maintained the key, performed the injection of anaesthetic, and then handed the mice in a random order to a second, blinded experimenter. The second experimenter performed the tail transections and measured the bleeding time. For most experiments, on day 1 the tail transection and bleeding time was measured. In some experiments, the experiment was performed on day 4 so that changes in reticulocytes and reticulated platelets could also be measured. However, the advantage of performing the measurement 1 day after treatment is that after only 24 hours, the level of circulating red blood cells will not have changed, so effects on blood clotting are due to direct effects on some element of the clotting system, and not due to changes in blood viscosity.

Tail transection was performed as follows. The mice were first anaesthetized using anaesthetics ketamine and xylazine. These anaesthetics are chosen because they are thought to not affect blood clotting. Acepromazine is not used because it has the effect of reducing clotting. Mice were weighed, and mice were then injected with 120-160 mg ketamine/kg and 10-16 mg xylazine/kg of body weight. For older and heavier mice, sometimes an additional injection of about 25% of the first injection was required. After a mouse became unresponsive to a stimulus such as significant pressure to a hind foot, the mouse was placed on a heated pad on a platform over a water bath. The water bath was maintained at 37° C. 50-mL blue-cap tubes (Sarstedt) were filled with 50 mL of a solution of 0.85 to 0.9% NaCl that has been equilibrated to 37° C. in a separate water bath. The animal was placed on a Chux pad for the transection.

A position on the tail that is 3 mm from the tip, not counting hair, was marked with a felt-tip pen using calipers. (The tail was also inspected for signs of bruising that may be due to fighting, and data from such a mouse was discarded if the transected tail did not bleed at all. The decision to discard the data was always made in a blinded manner at the time of measurement). The tail was transected with a flat razor blade using a section of the blade that has not been used previously. Within 1-2 seconds, the transected tail was placed in a tube with pre-warmed saline, and then the body of the mouse was placed on the heated pad above the water bath. At the moment that the tail was transected by the blinded experimenter, the non-blinded experimenter started a timer. The body of the mouse was then positioned on the heated pad so that only the tip of the tail—about 0.5 to 2 mm—is in the saline and the rest is in the air. When observing the bleeding tail, the tube was rotated so that the white stripe was behind the tail, providing contrast, and the room was well-lit. The rack holding the 50-mL tube was white or yellow to provide contrast. The bleeding time was recorded by noting when bleeding stops, and then observing the submerged tail for up to one minute. If bleeding re-started within this minute, the first recorded time was not counted. Bleeding may stop and re-start several times. If the tail is still bleeding when 10 minutes have elapsed, the time was recorded as 10 minutes.

The median and mean bleeding times were calculated for each treatment group. Calculating the median has the advantage that extreme events, such as 10-minute time points, do not disproportionately contribute to the calculation. Data from each experiment were normalized to the vehicle control in the same experiment to account for day-to-day variability. The normalized values were combined across experiments and Mann-Whitney test was performed to determine p-values the combined data set.

TABLE 4
Summary of ability of diverse forms of Targeted
EPO to stimulate proliferation of TF-1 cells in
vitro. Bold indicates data shown in FIG. 15B.

| Protein | | Activity in vitro | EC50 relative to |
|---|---|---|---|
| (V region-linker-EPO) | N | Log(EC50) ± SD | epoetin alpha |
| Epoetin alfa (Amgen) | 16 | −10.239 ± 0.345 | 1 |
| Darbepoetin (Amgen) | 8 | −8.989 ± 0.135 | 14.67 |
| EPO(WT) | 3 | −10.378 ± 0.528 | 0.87 |
| EPO(R150A) | 7 | −8.078 ± 0.233 | 130.86 |
| EPO(K45D) | 3 | −6.572 ± 0.754 | 7298 |
| 10F7-35-EPO(R150A) | 4 | −8.947 ± 0.206 | 16.71 |
| 10F7-29-EPO(R150A) | 6 | −9.320 ± 0.303 | 7.78 |
| 10F7-18-ERO(R150A) | 5 | −9.173 ± 0.297 | 10.89 |
| 10F7-17-EPO(R150A) | 6 | −9.163 ± 0.306 | 11.27 |
| 10F7-7-EPO(R150A) | 5 | −9.386 ± 0.253 | 6.39 |
| 10F7-5-EPO(R150A) | 8 | −9.378 ± 0.754 | 13.69 |
| 10F7-EPO(R150A)-HEL | 4 | −9.142 ± 0.501 | 16.02 |
| 10F7-EPO(R150A)-LEH | 4 | −8.648 ± 0.643 | 54.76 |
| 1C3-35-EPO(R150A) | 6 | −10.517 ± 0.471 | 0.81 |
| 1C3-EPO(R150A)-HEL | 4 | −10.682 ± 0.900 | 0.98 |
| 1C3-EPO(R150A)-LEH | 4 | −10.808 ± 0.527 | 0.30 |
| R18-17-EPO(R150A) | 2 | −9.415 ± 0.409 | 6.44 |
| R18-7-EPO(R150A) | 2 | −10.074 ± 0.690 | 1.95 |
| R18-5-EPO(R150A) | 2 | −9.495 ± 0.185 | 4.56 |
| R18-17-EPO(K45D) | 2 | −8.828 ± 0.786 | 39.24 |
| R18-7-EPO(K45D) | 1 | −8.494 ± N/A | 43.71 |
| R18-5-EPO(K45D) | 1 | −8.019 ± N/A | 130.58 |
| IH4-35-EPO(R150A) | 16 | −10.385 ± 0.756 | 1.45 |
| IH4-17-EPO(R150A) | 5 | −10.243 ± 0.505 | 1.34 |
| IH4-7-EPO(R150A | 5 | −10.479 ± 0.645 | 1.13 |
| IH4-5-EPO(R150A) | 9 | −10.410 ± 0.293 | 0.69 |
| IH4-35-EPO(K45D) | 5 | −9.591 ± 0.364 | 4.70 |
| IH4-5-EPO(K45D) | 1 | −9.559 ± N/A | 3.76 |

TABLE 5
Summary of epitopes and binding kinetics of anti-GPA antibody
fragments and EPO (wildtype and R150A mutant) studied in this work.

| | | | K_{D} | Antibody | GPA | |
|---|---|---|---|---|---|---|
| Protein | kₒₙ (M⁻¹ s⁻¹) | k_{off} (s⁻¹) | (nM) | form | epitope | References |
| 10F7 | — | — | 95 | Fab | ₃₄YAATP₃₈ | Chasis et al, 1988 |
| | | | | | | Chasis & |
| | | | | | | Mohandas, 1992 |
| | | | | | | Catimel et al., 1993 |
| 1C3 | — | — | 230 | Fab | — | Catimel et al., 1993 |
| | — | — | 62 | scFv | — | Patent Application |
| | | | | | | WO1994007921 |
| R18 | — | — | 25 | IgG | ₄₉RTVY₅₂ | Gardner et al., 1989 |
| | — | — | 400 | Fab | | |
| IH4 | 5.73 × 10⁵ | 0.019 | 33.72 | Nanobody | ₅₂YPPE₅₅ | Habib et al., 2013 |
| | | | | (VHH) | | |
| EPO (WT) | 3.9 × 10⁴ | 2.1 × 10⁻⁴ | 5.4 | N/A | N/A | Burrill et al., 2016 |
| EPO (R150A) | 4.2 × 10⁴ | 3.4 × 10⁻³ | 81 | N/A | N/A | Burrill et al., 2016 |

TABLE 6
A list of DNA sequences encoding anti-GPA antibody
fragments, linkers, and EPO.

| | Sequence | Reference |
|---|---|---|
| IH4 | CAGGTCCAACTGCAGGAGAGCGGCGGGGGGTCAGTTCAGGCGGGGGGGAGT | |
| nanobody | CTGCGGTTGAGCTGCGTAGCTTCAGGCTACACTGACAGCACCTACTGCGTG | |
| | GGATGGTTTCGGCAGGCACCCGGCAAGGAACGAGAGGGCGTTGCACGGATC | |
| | AACACTATCTCCGGTCGGCCTTGGTACGCAGATAGTGTTAAGGGACGGTTT | |
| | ACTATTAGTCAGGATAACTCTAAGAATACCGTCTACCTTCAGATGAATAGC | |
| | CTGAAACCGGAAGACACGGCTATTTACTATTGCACCCTTACAACTGCCAAC | |
| | AGCAGAGGGTTTTGTTCTGGGGGATATAACTACAAAGGACAGGGGACCCAA | |
| | GTCACTGTCAGC | |
| IH4* | CAGGTCCAACTGCAAGAGAGCGGAGGAGGGTCTGTTCAAGCTGGCGGTTCC | |
| nanobody | CTCCGGCTTTCTTGCGTGGCGTCAGGCTATACTGACAGCACATACTGCGTG | |
| | GGCTGGTTCAGGCAGGCCCCTGGAAAGGAGCGCGAGGGCGTAGCCCGCATA | |
| | AATACTATATCTGGCAGACCGTGGTACGCTGACAGCGTGAAGGGACGGTTT | |
| | ACAATCAGTCAAGATAACTCTAAAAACACCGTGTTTCTTCAAATGAATTCT | |
| | TTGAAACCCGAAGATACTGCCATCTATTATTGCACACTTACGACCGCGAAC | |
| | TCACGCGGTTTTTGTAGCGGAGGATATAACTATAAAGGGCAAGGGCAGGTA | |
| | ACTGTATCC | |
| 10F7 | CAAGTTAAGTTGCAACAATCTGGTGCTGAATTGGTTAAGCCAGGTGCTTCT | GenBank |
| scFv | GTTAAGTTGTCTTGTAAGGCTTCTGGTTACACCTTCAACTCTTACTTTATG | (accession |
| | CATTGGATGAAGCAAAGACCAGTTCAAGGTTTGGAATGGATTGGTATGATT | no. |
| | AGACCAAACGGTGGTACTACCGATTACAACGAGAAGTTTAAGAACAAGGCT | KX026660-3) |
| | ACTTTGACTGTTGATAAGTCCTCTAACACTGCTTACATGCAATTGAACTCT | |
| | TTGACTTCTGGTGATTCTGCTGTTTACTACTGTGCTAGATGGGAAGGTTCT | |
| | TACTACGCTTTGGATTACTGGGGTCAAGGTACCACTGTTACTGTTTCTTCC | |
| | GGTGGAGGTGGATCTGGTGGTGGAGGATCTTCAGGAGGTGGTGGATCTTCC | |
| | GATATTGAGTTGACTCAATCTCCAGCTATTATGTCTGCTACCTTGGGTGAG | |
| | AAGGTTACTATGACTTGTAGAGCTTCATCTAACGTTAAGTACATGTACTGG | |
| | TACCAACAGAAGTCTGGTGCTTCTCCAAAGTTGTGGATTTACTACACTTCT | |
| | AACTTGGCTTCTGGTGTTCCAGGTAGATTTTCTGGTTCAGGTTCTGGTACT | |
| | TCCTACTCTTTGACTATTTCCTCTGTTGAAGCTGAAGATGCTGCTACTTAC | |
| | TACTGTCAACAATTCACTTCTTCCCCATACACTTTTGGAGGAGGTACTAAG | |
| | TTGGAAATCAAG | |
| 1C3 | GAAGTCCGTCTGCTGGAAAGCGGGGGTGGTCCTGTGCAGCCTGGTGGGTCC | |
| scFv | CTGAAACTGTCCTGTGCCGCAAGCGGGTTCGATTTTTCCAGATACTGGATG | |
| | AACTGGGTGAGGAGGGCTCCAGGCAAGGGCCTGGAGTGGATCGGCGAGATC | |
| | AACCAGCAGTCCAGCACCATCAATTACTCTCCCCCTCTGAAGGACAAGTTC | |
| | ATCATCAGCCGCGATAACGCTAAGTCTACACTGTATCTGCAGATGAATAAG | |
| | GTGAGAAGCGAGGACACCGCCCTGTACTATTGCGCTCGCCTGTCTCTGACA | |
| | GCCGCTGGCTTTGCCTATTGGGGCCAGGGCACCCTGGTGACAGTGTCTGCT | |
| | GGAGGAGGCTCTTCCGGAGGATCCGGCAGCTCTGGCGGCTCCAGCTCTGGC | |
| | GGCGATATCGTGATGAGCCAGTCTCCCTCCAGCCTGGCCGTGTCCGTGGGA | |
| | GAGAAGGTGTCCATGAGCTGTAAGTCTTCCCAGTCTCTGTTCAACTCCAGA | |
| | ACCCGCAAGAATTACCTGACATGGTATCAGCAGAAGCCTGGCCAGAGCCCC | |
| | AAGCCTCTGATCTACTGGGCCAGCACCAGAGAGTCTGGAGTGCCAGACCGC | |
| | TTCACCGGCTCTGGATCCGGCACAGACTTCACCCTGACAATCAGCTCTGTG | |
| | CAGGCCGAGGACCTGGCTGATTACTATTGCAAGCAGTCCTATAATCTGAGG | |
| | ACCTTTGGCGGCGGCACAAAGCTGGAGATCAAG | |
| R18 | CAGGTTAAACTCCAGCAAAGTGGTGGCGGGCTCGTACAACCAGGCGGTTCC | |
| scFv | CTCAAGTTGTCCTGCGCCGCATCAGGGTTTACATTTAGCTCTTATGGTATG | |
| | TCTTGGTTTCGCCAGACGCCTGACAAGCGACTCGAGCTGGTCGCTATCATC | |
| | AATAGTAACGGAGGTACTACATATTATCCCGACAGTGTGAAGGGGCGATTT | |
| | ACCATTAGCCGGGACAACGCCAAAAATACACTGTACCTCCAGATGTCAAGC | |
| | TTGAAATCAGAAGATACGGCCATGTACTATTGCGCTAGGGGGGGTGGAAGG | |
| | TGGCTTCTGGACTATTATGGTCAGGGTACAACAGTGACAGTATCCTCCGGT | |
| | GGAGGTGGATCTGGTGGTGGAGGATCTTCAGGAGGTGGTGGATCTTCCGAC | |
| | ATAGAGCTTACACAATCTCCGTCATCACTGGCAGTCTCAGCCGGGGAAAAA | |
| | GTGACAATGTCATGCAAGTCAAGCCAGAGCGTTCTTTATTCATCTAATCAG | |
| | AAGAACTACCTGGCATGGTATCAGCAGAAGCCGGGACAGTCCCCTAAGCTC | |
| | CTCATCTACTGGGCAAGCACCAGGGAATCCGGAGTGCCGGACAGGTTTACT | |
| | GGGTCCGGTTCTGGGACGGATTTTACGCTTACGATATCAAGTGTCCAAGCT | |
| | GAGGACCTCGCAGTATACTACTGTCACCAGTACCTGTCTTCTTCTACTTTT | |
| | GGGGGTGGAACGAAACTGGAAATAAAA | |
| EPO | GCTCCACCTAGATTGATTTGTGATTCCAGAGTTTTGGAAAGATACTTGTTG | GenBank |
| (WT) | GAAGCTAAGGAGGCTGAAAATATTACTACTGGTTGTGCTGAACATTGTTCT | (accession |
| | TTGAACGAGAATATTACTGTTCCAGATACTAAGGTTAACTTTTACGCTTGG | no. |
| | AAGAGAATGGAAGTTGGTCAGCAAGCTGTTGAAGTTTGGCAAGGTTTGGCT | KX026660-3) |
| | TTGTTGTCTGAAGCTGTTTTGAGAGGTCAAGCTTTGTTGGTTAATTCTTCT | |
| | CAACCATGGGAACCATTGCAATTGCATGTTGATAAGGCTGTTTCTGGTTTG | |
| | AGATCTTTGACTACCTTGTTGAGAGCTTTGGGTGCTCAAAAGGAAGCTATT | |
| | TCTCCTCCAGATGCTGCTTCTGCCGCTCCATTGAGAACTATTACTGCTGAT | |
| | ACTTTTAGAAAGTTGTTTAGAGTTTACTCTAACTTCTTGAGAGGTAAGTTG | |
| | AAGTTGTACACTGGTGAAGCTTGTAGAACTGGTGATCGG | |
| EPO | GCTCCACCTAGATTGATTTGTGATTCCAGAGTTTTGGAAAGATACTTGTTG | GenBank |
| (R150A) | GAAGCTAAGGAGGCTGAAAATATTACTACTGGTTGTGCTGAACATTGTTCT | (accession |
| | TTGAACGAGAATATTACTGTTCCAGATACTAAGGTTAACTTTTACGCTTGG | no. |
| | AAGAGAATGGAAGTTGGTCAGCAAGCTGTTGAAGTTTGGCAAGGTTTGGCT | KX026660-3) |
| | TTGTTGTCTGAAGCTGTTTTGAGAGGTCAAGCTTTGTTGGTTAATTCTTCT | |
| | CAACCATGGGAACCATTGCAATTGCATGTTGATAAGGCTGTTTCTGGTTTG | |
| | AGATCTTTGACTACCTTGTTGAGAGCTTTGGGTGCTCAAAAGGAAGCTATT | |
| | TCTCCTCCAGATGCTGCTTCTGCCGCTCCATTGAGAACTATTACTGCTGAT | |
| | ACTTTTAGAAAGTTGTTTAGAGTTTACTCTAACTTCTTGGCCGGTAAGTTG | |
| | AAGTTGTACACTGGTGAAGCTTGTAGAACTGGTGATCGG | |
| EPO | GCTCCACCTAGATTGATTTGTGATTCCAGAGTTTTGGAAAGATACTTGTTG | GenBank |
| (K45D) | GAAGCTAAGGAGGCTGAAAATATTACTACTGGTTGTGCTGAACATTGTTCT | (accession |
| | TTGAACGAGAATATTACTGTTCCAGATACTGATGTTAACTTTTACGCTTGG | no. |
| | AAGAGAATGGAAGTTGGTCAGCAAGCTGTTGAAGTTTGGCAAGGTTTGGCT | KX026660-3) |
| | TTGTTGTCTGAAGCTGTTTTGAGAGGTCAAGCTTTGTTGGTTAATTCTTCT | |
| | CAACCATGGGAACCATTGCAATTGCATGTTGATAAGGCTGTTTCTGGTTTG | |
| | AGATCTTTGACTACCTTGTTGAGAGCTTTGGGTGCTCAAAAGGAAGCTATT | |
| | TCTCCTCCAGATGCTGCTTCTGCCGCTCCATTGAGAACTATTACTGCTGAT | |
| | ACTTTTAGAAAGTTGTTTAGAGTTTACTCTAACTTCTTGAGAGGTAAGTTG | |
| | AAGTTGTACACTGGTGAAGCTTGTAGAACTGGTGATCGG | |
| 5AA | TCTGGTGGTGGTTCC | |
| 7AA | GGAGGATCTGGTGGTGGTTCC | |
| 17AA | GGAGGATCCGGTGGTGGAGGATCATCTGGTGGAGGATCTGGTGGTGGTTCC | |
| 29AA | GGAGGAAGTTCCGGTGGTGGATCTTCTTCTGGAGGTGGAGGATCCGGTGGT | |
| | GGAGGATCATCTGGTGGAGGATCTGGTGGTGGTTCC | |
| 35AA | GGTGGAGGTGGTTCCGGAGGAGGAAGTTCCGGTGGTGGATCTTCTTCTGGA | |
| | GGTGGAGGATCCGGTGGTGGAGGATCATCTGGTGGAGGATCTGGTGGTGGT | |
| | TCC | |

TABLE 7
A list of protein sequences for anti-GPA antibody
fragments, linkers, and EPO.

| | Sequence | Reference |
|---|---|---|
| IH4 | QVQLQESGGGSVQAGGSLRLSCVASGYTDSTYCVGWFRQAPGKEREGVARI | U.S. Pat. No. |
| nanobody | NTISGRPWYADSVKGRFTISQDNSKNTVYLQMNSLKPEDTAIYYCTLITAN | 9,879,090¹ |
| | SRGFCSGGYNYKGQGTQVTVS | |
| IH4* | QVQLQESGGGSVQAGGSLRLSCVASGYTDSTYCVGWFRQAPGKEREGVARI | U.S. Pat. No. |
| nanobody | NTISGRPWYADSVKGRFTISQDNSKNTVFLQMNSLKPEDTAIYYCTLITAN | 9,879,090¹ |
| | SRGFCSGGYNYKGQGQVTVS | |
| 10F7 | QVKLQQSGAELVKPGASVKLSCKASGYTFNSYFMHWMKQRPVQGLEWIGMI | |
| scFv | RPNGGTTDYNEKFKNKATLTVDKSSNTAYMQLNSLTSGDSAVYYCARWEGS | |
| | YYALDYWGQGTTVTVSSGGGGSGGGGSSGGGGSSDIELTQSPAIMSATLGE | |
| | KVTMTCRASSNVKYMYWYQQKSGASPKLWIYYTSNLASGVPGRFSGSGSGT | |
| | SYSLTISSVEAEDAATYYCQQFTSSPYTFGGGTKLEIK | |
| 1C3 | EVRLLESGGGPVQPGGSLKLSCAASGFDFSRYWMNWVRRAPGKGLEWIGEI | Patent |
| scFv | NQQSSTINYSPPLKDKFIISRDNAKSTLYLQMNKVRSEDTALYYCARLSLT | application |
| | AAGFAYWGQGTLVTVSAGGGSSGGSGSSGGSSSGGDIVMSQSPSSLAVSVG | WO19930246302 |
| | EKVSMSCKSSQSLFNSRTRKNYLTWYQQKPGQSPKPLIYWASTRESGVPDR | |
| | FTGSGSGTDFRLTISSVQAEDLADYYCKQSYNLRTFGGGTKLEIK | |
| R18 | QVKLQQSGGGLVQPGGSLKLSCAASGFTFSSYGMSWFRQTPDKRLELVAII | U.S. Pat. No. |
| scFv | NSNGGTTYYPDSVKGRFTISRDNAKNTLYLQMSSLKSEDTAMYYCARGGGR | 8,900,592⁸ |
| | WLLDYYGQGTTVTVSSGGGGSGGGGSSGGGGSSDIELTQSPSSLAVSAGEK | |
| | VTMSCKSSQSVLYSSNQKNYLAWYQQKPGQSPKLLIYWASTRESGVPDRFT | |
| | GSGSGTDFTLTISSVQAEDLAVYYCHQYLSSSTFGGGTKLEIK | |
| EPO | APPRLICDSRVLERYLLEAKEAENITTGCAEHCSLNENITVPDTKVNFYAW | |
| (WT) | KRMEVGQQAVEVWQGLALLSEAVLRGQALLVNSSQPWEPLQLHVDKAVSGL | |
| | RSLTTLLRALGAQKEAISPPDAASAAPLRTITADTFRKLFRVYSNFLRGKL | |
| | KLYTGEACRTGDR | |
| EPO | APPRLICDSRVLERYLLEAKEAENITTGCAEHCSLNENITVPDTKVNFYAW | |
| (R150A) | KRMEVGQQAVEVWQGLALLSEAVLRGQALLVNSSQPWEPLQLHVDKAVSGL | |
| | RSLTTLLRALGAQKEAISPPDAASAAPLRTITADTFRKLFRVYSNFLAGKL | |
| | KLYTGEACRTGDR | |
| EPO | APPRLICDSRVLERYLLEAKEAENITTGCAEHCSLNENITVPDTDVNFYAW | |
| (K45D) | KRMEVGQQAVEVWQGLALLSEAVLRGQALLVNSSQPWEPLQLHVDKAVSGL | |
| | RSLTTLLRALGAQKEAISPPDAASAAPLRTITADTFRKLFRVYSNFLRGKL | |
| | KLYTGEACRTGDR | |
| 5AA | SGGGS | |
| 7AA | GGSGGGS | |
| 17AA | GGSGGGGSSGGGSGGGS | |
| 29AA | GGSSGGGSSSGGGGSGGGGSSGGGSGGGS | |
| 35AA | GGGGSGGGSSGGGSSSGGGGSGGGGSSGGGSGGGS | |

### REFERENCES

[1] Bertrand, O., Habib, I., Kremlin-Bicetre, L., and Smolarek, D. (2018) Fusion proteins and immunoconjugates and uses thereof which are specific for glycophorin A, INSERM.
[2] Lilley, G. G., Hudson, P. J., and Hillyard, C. J. (1993) Reagent for agglutination assays, C12N 15/13, 15/70, C07K 15/12, 15/28, G01N 33/563, 33/577 ed.
[3] Cohen, J. H. M., Mahmoud, W., Libyh, M. T., Godin, N., Gimenez, A., Tabary, T., Donvito, B., Baty, D., and Dervillez, X. (2014) Protein constructs designed for targeting and lysis of cells, Universite de Reims Champagne, Ardenne, Reims(FR).
[4] Burrill, D. R., Vernet, A., Collins, J. J., Silver, P. A., and Way, J. C. (2016) Targeted erythropoietin selectively stimulates red blood cell expansion in vivo, Proc Nat Acad Sci USA 113, 5245-5250.
[5] Auffray, I., Marfatia, S., de Jong, K., Lee, G., Huang, C.-H., Paszty, C., Tanner, M. J. A., Mohandas, N., and Chasis, J. A. (2001) Glycophorin A dimerization and band 3 interaction during erythroid membrane biogenesis: in vivo studies in human glycophorin A transgenic mice, Blood 97, 2872-2878.
[6] Khoory, J., Estanislau, J., Elkhal, A., Lazaar, A., Melhorn, M. I., Brodsky, A., Illigens, B., Hamachi, I., Kurishita, Y., Ivanov, A. R., Shevkoplyas, S., Shapiro, N. I., and Ghiran, I. C. (2016) Ligation of Glycophorin A Generates Reactive Oxygen Species Leading to Decreased Red Blood Cell Function, PLoS One 11, e0141206.
[7] Melhorn, M. I., Brodsky, A. S., Estanislau, J., Khoory, J. A., Illigens, B., Hamachi, I., Kurishita, Y., Fraser, A. D., Nicholson-Weller, A., Dolmatova, E., Duffy, H. S., and Ghiran, I. C. (2013) CR1-mediated ATP release by human red blood cells promotes CR1 clustering and modulates the immune transfer process, J Biol Chem 288, 31139-31153.
[8] Cohen, J. H. M., Mahmoud, W., Libyh, M. T., Codin, N., Gimenez, A., Tabary, T., Donvito, B., Baty, D., and Dervillez, X. (2014) Protein constructs designed for targeting and lysis of cells, Universite de Reims Champagne Ardenne.

### Example 13: Rational Design of a Bifunctional AND-Gate Ligand to Modulate Cell-Cell Interactions

Protein “AND-gate” systems, in which a ligand acts only on cells with two different receptors, direct signaling activity to a particular cell type and avoid action on other cells. In a bifunctional AND-Gate protein, the molecular geometry of the protein domains is crucial. Here we constructed a tissue-targeted erythropoietin (EPO) that stimulates red blood cell (RBC) production without triggering thrombosis. EPO was directed to RBC precursors and mature RBCs by fusion to an anti-glycophorin A antibody V region. Many such constructs activated EPO receptors in vitro and stimulated RBC and not platelet production in mice but nonetheless enhanced thrombosis in mice, and caused adhesion between RBCs and EPO receptor-bearing cells. Based on a protein-structural model of the RBC surface, described herein is a rationally designed anti-glycophorin/EPO fusion that does not induce cell adhesion in vitro or enhance thrombosis in vivo. Thus, meso-scale geometry can inform design of synthetic-biological systems.

Hormones and cytokines act on receptors on diverse cells to achieve coordinated biological responses. Some activities are desired while others constitute drug side effects. For example, cortisol acts on the glucocorticoid receptor in immune cells and is anti-inflammatory, but acts on other cells to increase blood sugar and induce bone resorption. Similarly, erythropoietin (EPO) stimulates red blood cell (RBC) production but also increases thrombotic activity. These responses are adaptive in the context of hemorrhage, but increased thrombotic activity may underlie the increased frequency of heart attacks and strokes seen in EPO-treated kidney failure patients.

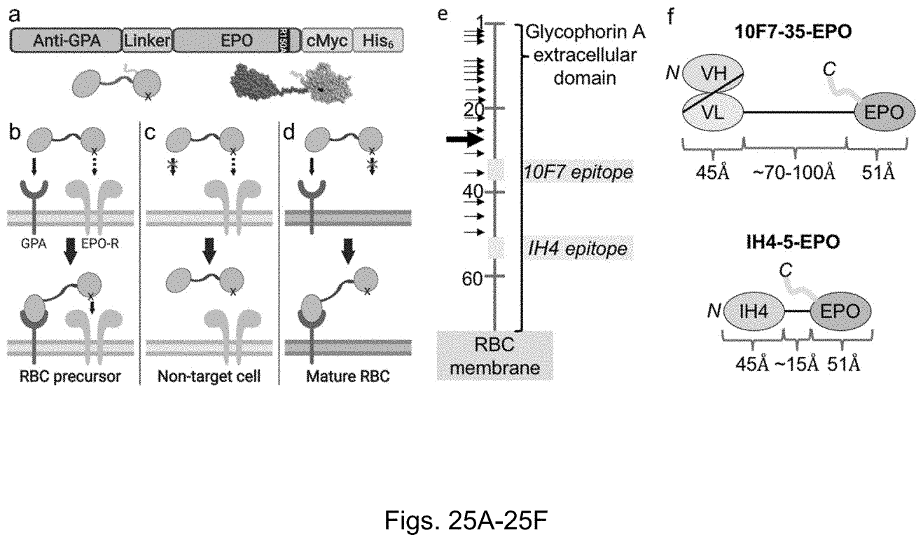

A class of AND-Gate proteins, “Chimeric Activators,” was previously designed in which a cytokine or hormone is mutated to reduce its activity, and is attached to a targeting element that binds to another protein on a subset of receptor-bearing cells (FIG. 25A-25D). Chimeric activators are similar to other fusion proteins such as blinatumomab¹ or immunocytokines,² which connect one cell to another. Thus, implicit in this design is the potential to crosslink cells, whether desired or not. Understanding the structural determinants for engineered cell-cell interaction will be important in design of next-generation CAR-T cells, bispecific T-cell engagers, and other fusion proteins.³

An AND-gated EPO was constructed that activates EPO receptors (EPO-Rs) on RBC precursors but not on other cells.^{4,5}“Targeted EPO” proteins consist of an antibody element that binds to glycophorin A (GPA), EPO with a mutation that reduces EPO activity, and a connecting linker (FIG. 25A).^{4,5} GPA is abundant on RBCs (800,000 copies/cell) and late RBC precursors (50,000 copies/cell).^{4,6} GPA is not expressed on cells that control thrombosis and tumor angiogenesis, or solid tumor cells. EPO-Rs on late RBC precursors mediate EPO-dependent maturation, but then disappear.^{4,7} Targeted EPO was designed to bind to late RBC precursors and stimulate EPO-Rs on only those cells (FIG. 25B). On non-target cells, it fails to activate EPO-Rs because the mutated EPO alone binds poorly to receptors (FIG. 25C). Binding to GPA on mature RBCs is a desirable secondary consequence as it extends the plasma half-life of the fusion protein.⁴

The previously characterized form of Targeted EPO had a long linker between the antibody and EPO elements, and an anti-GPA antibody element that binds to a membrane-distal epitope. That protein stimulated production of RBCs but not platelets, the latter being considered an indicator of off-target cell effects. While this result indicated cell-type specificity of action, platelet number alone is not strongly correlated with thrombotic propensity, and the possibility remained that the fusion protein might still cause thrombosis despite its seeming cell-type specificity. In this work it was found, surprisingly, that the initial fusion protein might have a thrombotic activity in vivo. The structure-function analysis presented here indicates that, depending on the Targeted EPO configuration, the EPO moiety tethered to mature RBCs could interact with EPO-Rs on other cells and lead to cell adhesion, but that the potential for such interaction could be eliminated by rational design. In vivo, a poorly designed fusion protein could bind to both RBCs and endothelial cells⁸ or immune cells such as macrophages⁹ and mediate undesired cell-cell adhesion or signaling (FIG. 25D).⁴

Results and Discussion

The present work defines structural features of anti-GPA/EPO chimeric activators designed to act specifically in cis on GPA-expressing cells, to avoid side effects that may arise from unwanted cell-cell interaction. Previously a form of Targeted EPO consisting of V regions from the 10F7 antibody, a 35-amino acid linker, and EPO with the weakening mutation R150A; was characterized. This version is termed 10F7-35-EPO(R150A) (FIG. 25F). This protein specifically stimulated production of RBCs but not platelets, the latter being considered an indicator of off-target cell effects. To directly assess the impact of this molecule on thrombosis, a ‘bleeding time’ assay was performed in which the tail of a treated mouse is transected and the time to cessation of bleeding is measured.

Contrary to expectation, the 10F7-35-EPO(R150A) form of Targeted EPO promoted thrombosis in the bleeding time assay (FIG. 31A-31C). It was hypothesized that there were two mechanisms by which this might occur. First, the antibody element could, by itself, promote blood clotting. Second, the RBC-bound Targeted EPO could crosslink RBCs with other cells bearing EPO-Rs, such as vascular endothelial cells or leukocytes, slowing blood flow. To determine whether either or both of these hypotheses were correct, a series of Targeted EPO variants were constructed, testing anti-GPA antibody elements (10F7, 1C3, R18 and IH4), fusion protein configurations, and linker lengths.

Targeted EPO Activates EPO-Rs in a GPA-Dependent Manner In Vitro and In Vivo

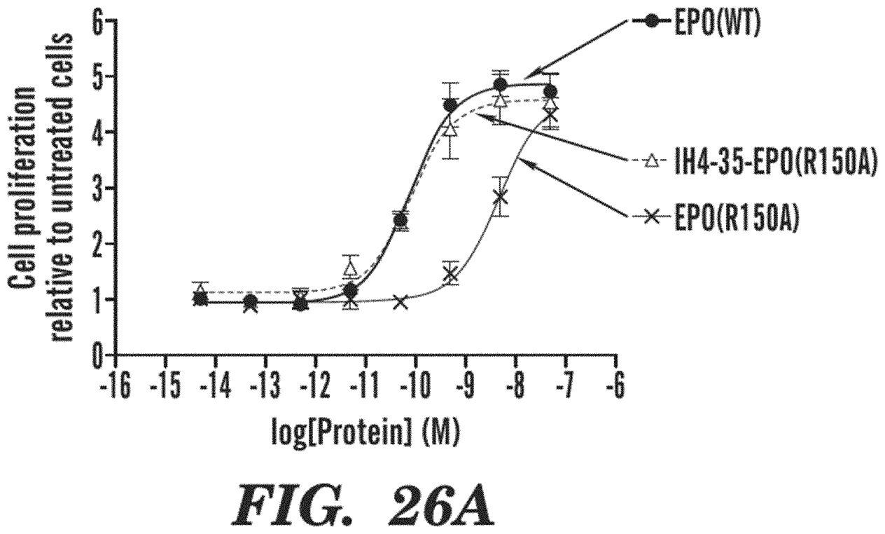

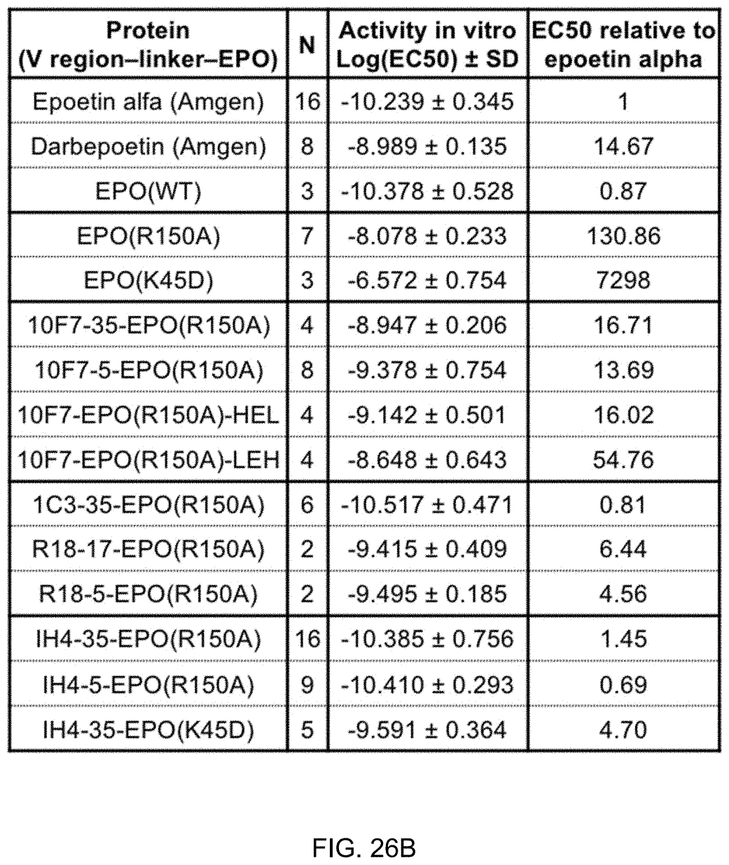

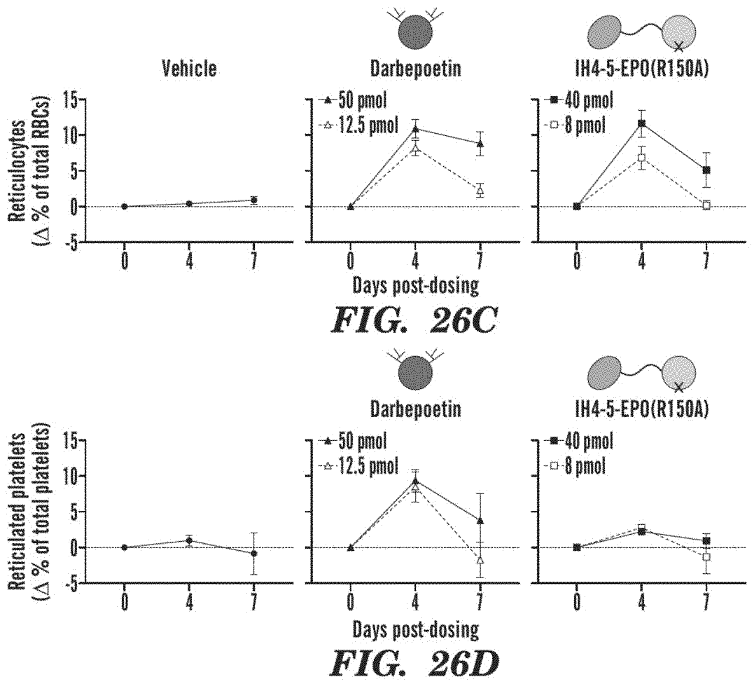

Diverse forms of Targeted EPO activate EPO-Rs in a GPA-dependent manner in vitro. TF-1 cells, which express both GPA and EPO-Rs,⁵ were treated with several forms of Targeted EPO, and their proliferation was measured 72 hr post-treatment (FIG. 26A, 26B). Unfused EPO(R150A) showed about 100-fold reduced activity compared to wild-type EPO (EPO(WT)). When EPO(R150A) was fused to an anti-GPA antibody element, its activity was rescued by about 10-100-fold (FIG. 26A, 26B). IH4 and 1C3 were the most potent antibody elements in these tests.

Three configurations of Targeted EPO were tested to address whether restricting the movement of EPO would affect EPO activity and potentially cause GPA-bound EPO to preferentially act in cis on targeted cells. The conventional scFv-EPO configuration is designed as antibody heavy chain—light chain—EPO (“HLE”). It was modified such that EPO would be between the heavy and light chains; this is feasible because the N- and C-termini of EPO are close in the folded structure. The resulting configurations (“HEL” and “LEH”) were expressed and tested. Both 10F7-EPO(R150A) and 1C3-EPO(R150A) stimulated proliferation of TF-1 cells largely independently of the configuration, indicating that orienting EPO in one way does not significantly constrain its receptor binding in cis (FIG. 26B and Table 4).

The GPA-mediated enhancement of EPO(R150A) activity was independent of the linker length in the various fusion proteins. Changing the linker of 10F7-EPO(R150A), R18-EPO(R150A), R18-EPO(K45D), IH4-EPO(R150A), and IH4-EPO(K45D) from 5 to 35 amino acids (about 10-80 Angstroms) in fusion proteins containing the same antibody and EPO elements showed similar EC50s in cell-based assays (FIG. 26B and Table 4). Results with the rotationally constrained and short-linker constructs are consistent with the idea that GPA is an intrinsically disordered, highly flexible protein, so that diverse fusion proteins bound to GPA can still allow binding of EPO with EPO-R.

When tested in vivo, various forms of Targeted EPO specifically stimulated production of RBCs but not platelets. In vivo target cell specificity and efficacy of several Targeted EPO proteins were assessed in mice that are transgenic for human GPA. Darbepoetin, an extended half-life form of EPO, is a control to represent non-targeted EPO activity. Mice were injected intraperitoneally with vehicle, darbepoetin, or a Targeted EPO, and their reticulocytes and reticulated platelets were measured to indicate activity on target cells and non-target cells, respectively. Mice treated with darbepoetin showed comparable increases in both new RBCs and new platelets (FIGS. 26C, 26D and 21).⁴ Mice treated with IH4-5-EPO(R150A) showed a similar increase in reticulocytes while the reticulated platelets were not significantly changed (FIGS. 26C, 26D). IH4-5-EPO(R150A) was the most potent in vivo of the forms of Targeted EPO tested (FIGS. 26B and 21).⁴

The Antibody Element IH4 does not Induce ‘RBC Inflammation’

The anti-GPA antibody element in forms of Targeted EPO might induce an inflammatory phenotype that could enhance blood clotting or cause other undesired side effects and also confound interpretation of experimental treatment with fusion proteins. Various anti-GPA antibodies and V region elements induce a constellation of effects on RBCs, including increased stiffness during shear stress, decreased membrane fluidity, secretion of ATP, production of reactive oxygen species, and phosphorylation of Tyr8 of band 3. Divalent IgG-type antibodies against GPA induce at least some and likely all of these phenotypes, but only a subset of monovalent Fabs, scFvs and VHHs induce this RBC response in an epitope-dependent manner (FIG. 25E).¹⁰⁻¹⁵

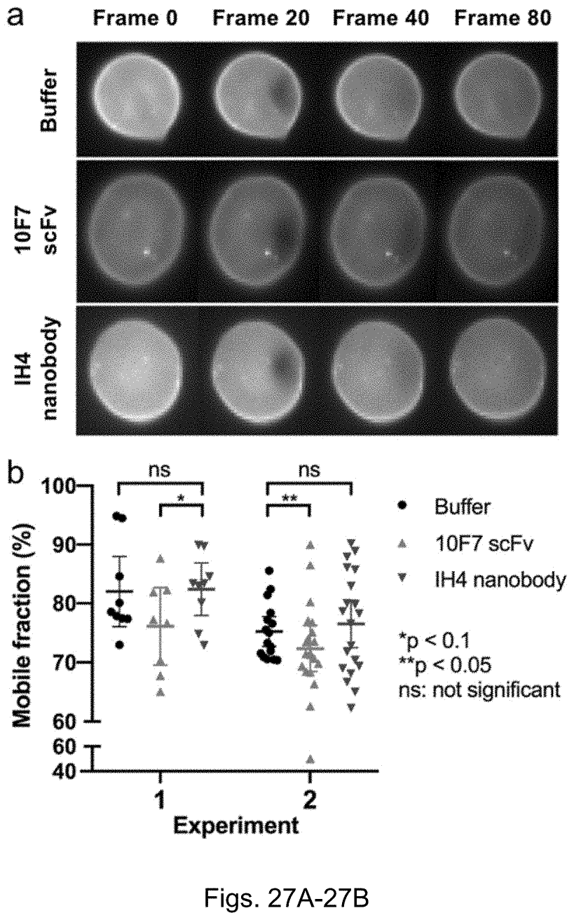

The single-chain camelid element (a VHH or nanobody) IH4 itself lacks stiffening/inflammatory activity when it is bound to GPA, and therefore is a good candidate targeting element.¹³ This element binds to the membrane-proximal epitope (₅₂YPPE₅₅) on human GPA.¹⁴ An optimized variant of IH4 was tested (FIG. 22A-22B). It was confirmed by fluorescence recovery after photobleaching (FRAP) that 10F7 induced lower membrane lipid mobility in RBCs, while IH4 did not (FIGS. 27A-27B). In addition, IH4 appears to bind most tightly of the known anti-GPA V region elements. Therefore, IH4 was chosen as a candidate antibody element for a Targeted EPO that would not induce undesired signaling and would also limit potential interaction of EPO with EPO-Rs on other cells.

Geometry of GPA and Targeted EPO Determines Potential for Cell Crosslinking

In the design of Targeted EPO, the intention is that the antibody element and EPO bind in cis to receptors on the same cell surface (FIG. 25B). However, fusion proteins comprising two binding elements could crosslink different cells that each bears a receptor for a component of the fusion protein. To address this, we tested the accessibility of antibodies to the EPO element of Targeted EPO variants bound to RBCs, and the ability of different Targeted EPOs to promote binding between RBCs and tumor cells expressing EPO-Rs.

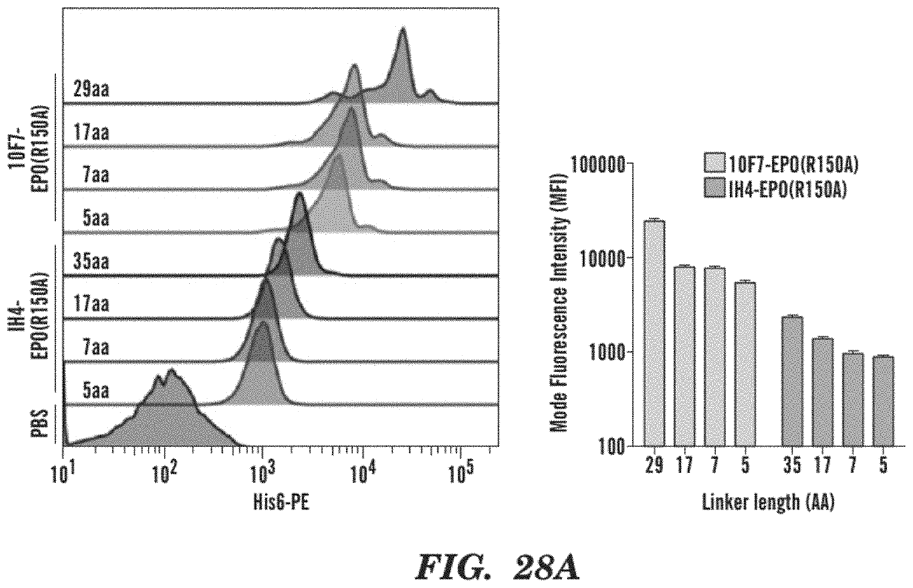

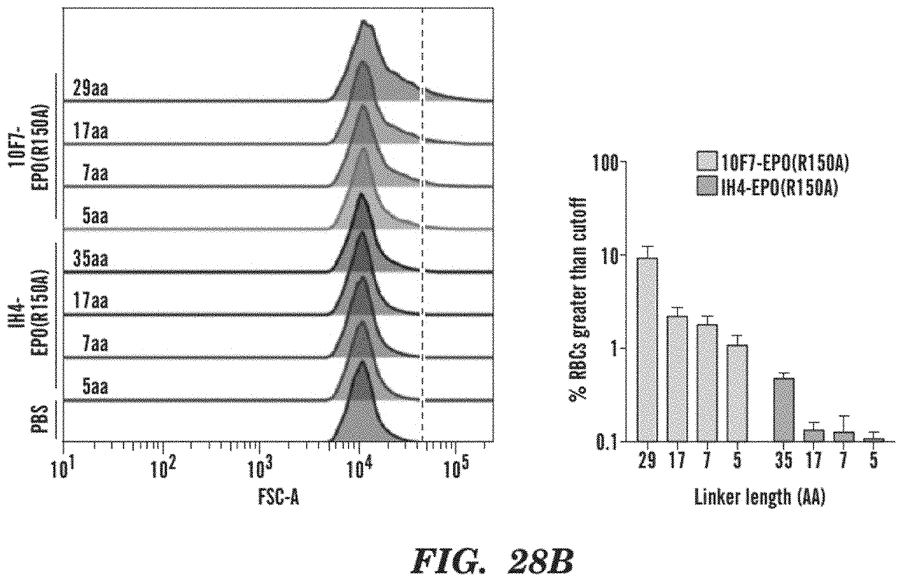

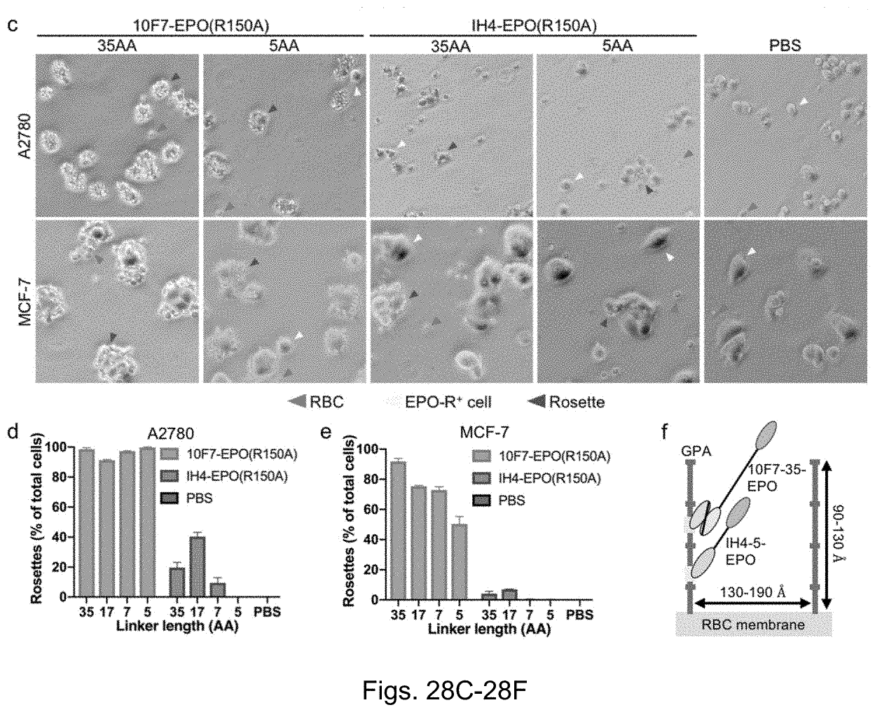

When forms of Targeted EPO are bound to RBCs via GPA, the antibody accessibility of the EPO element decreases when the linker length is shortened and the epitope on GPA is closer to the cell membrane. A series of proteins were compared, in which His₆-tagged EPO was fused to IH4 or 10F7 and the linker length was varied. RBCs were incubated with the fusion protein, and then the accessibility of bound EPO was assessed via flow cytometry by binding of a phycoerythrin (PE)-conjugated anti-His₆ antibody. For both IH4 and 10F7 fusion proteins, PE signals progressively decreased as the linker was shortened (FIG. 28A-28F). In addition, crosslinking of RBCs by the divalent anti-His₆ antibody increased with the linker length of the fusion protein, as seen in forward scattering signals evidenced by a shoulder to the right of the main peak (FIG. 28B). Regardless of the linker length, IH4 fusion proteins had lower accessibility and fewer RBC crosslinking events by anti-His₆ antibody than 10F7 fusion proteins, suggesting that the binding epitope of the antibody element also affects the accessibility of EPO (FIGS. 28A, 28B).

Forms of Targeted EPO with long linkers and a membrane-distal epitope on GPA mediate adhesion in vitro between RBCs and cells expressing EPO-Rs. A rosette assay was used for cell-cell interaction to address whether the anti-GPA/EPO fusion proteins promote adherence of RBCs to cells from the tumor lines A2780 and MCF-7, which express EPO-R.^{17,18} All forms of Targeted EPO containing the 10F7 scFv (epitope at about ₃₄YAATP₃₈) showed a high level of rosette formation, with most or all of the tumor cells binding to 3 or more RBCs. In contrast, rosette formation with Targeted EPOs containing the IH4 nanobody (epitope: ₅₂YPPE₅₅) was much lower. Targeted EPO forms with shorter linkers generally induced less rosette formation. These effects were synergistic: the IH4-EPO(R150A) with a 5 amino acid linker showed essentially no rosette formation by either A2780 or MCF-7 cells (FIGS. 28C-28E and FIG. 23A-23B).

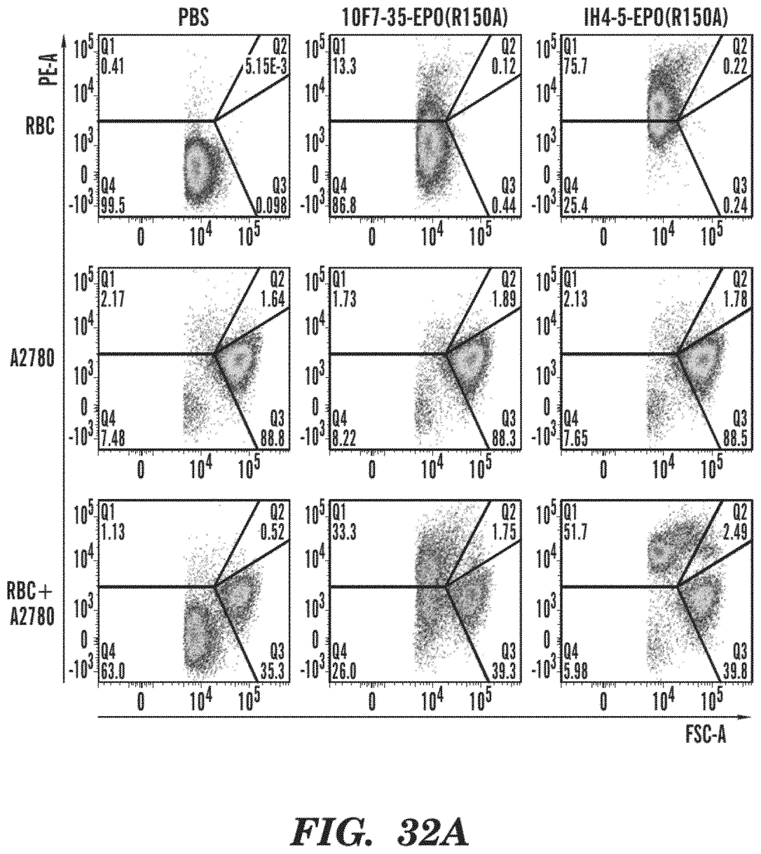

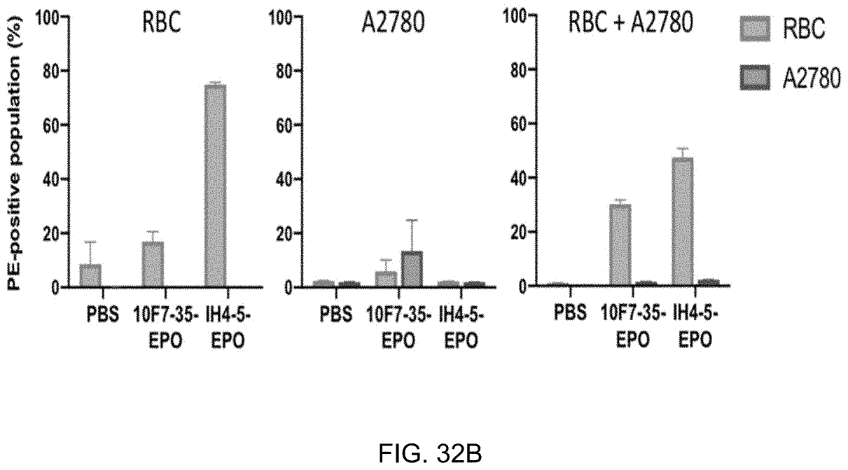

The results on antibody accessibility and rosette formation are consistent with a geometric model of the RBC surface in which GPA extends from the cell and shields the cell from interactions with other cells. There are about 800,000 GPA monomers⁶ in a surface area of 140 μm^{2,19} such that the average distance between GPA molecules is about 130-190 Angstroms (FIG. 28F and FIGS. 32A-32B). The membrane-distal N-terminal ˜45 amino acids of GPA include 17 sites for O- and N-glycosylation, with oligosaccharides that extend at least about 20 Angstroms from the peptide backbone (for the O-type blood group antigen).²⁰ It is thought that the N-terminal 45 amino acids of GPA are largely unstructured and would be about 90-130 Angstroms long in an extended conformation.²¹ By comparison, in an scFv-(SGGGS)-EPO fusion protein, the components would be 45, 15 and 51 Angstroms end-to-end. Thus, contact between a GPA-bound IH4-(SGGGS)-EPO molecule and IgG antibodies or EPO-Rs on another cell should be sterically very difficult (FIG. 28F).

IH4-5-EPO(R150A) does not Shorten Tail Vein Bleeding Time in Mice

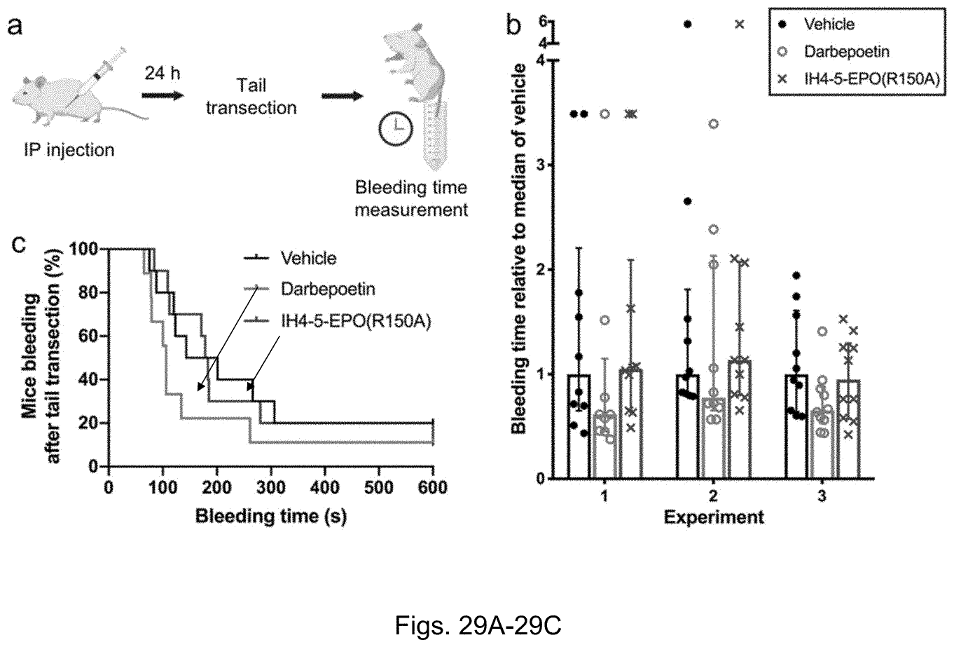

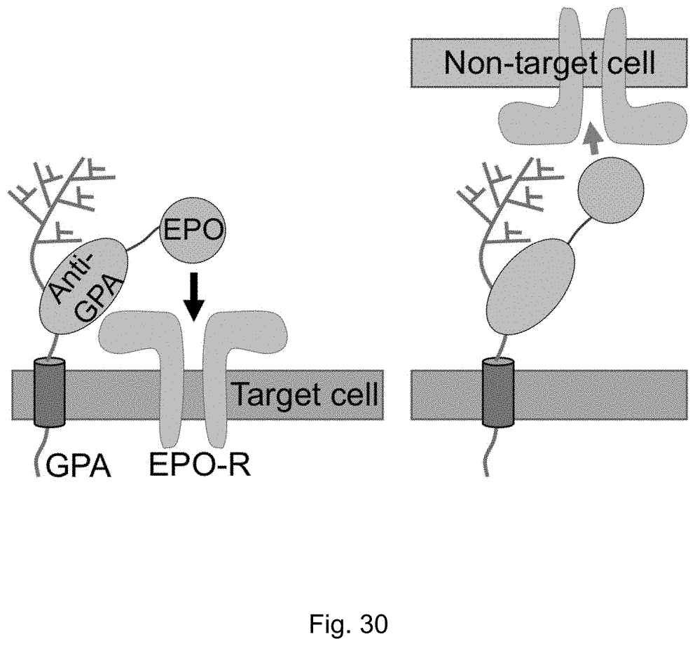

IH4-5-EPO(R150A) was tested for systemic effects on thrombosis in mice. HuGPA-transgenic mice were injected ip with vehicle, darbepoetin, 10F7-35-EPO(R150A) or IH4-5-EPO(R150A) (FIG. 29A). EPO proteins were injected at a dose that resulted in similar effect in mice, as measured by reticulocyte counts. In mice treated with darbepoetin or 10F7-35-EPO(R150A), the bleeding time was shortened by about 30%, whereas mice treated with IH4-5-EPO(R150A) showed no change in bleeding time (FIGS. 29B, 29C, and 31A-31C). These results indicate that IH4-5-EPO(R150A) does not enhance thrombotic side effects, in contrast to the non-targeted form EPO that acts on EPO-Rs on multiple cell types. When the data from these experiments were normalized to the vehicle controls of the same experiments and then combined, the difference between vehicle and darbepoetin (p=0.0098) or IH4-5-EPO(R150A) and darbepoetin (p=0.017) was significant. When data were compared across all experiments with dose groups including 10F7-35-EPO(R150A) and epoetin alfa, these molecules also showed a statistically significant decrease in bleeding time relative to the vehicle control (FIGS. 31A-31C). These results are consistent with previous studies indicating that non-targeted EPO proteins reduce bleeding time in rodents.²²

The design of a fusion protein must consider the spatial orientation of the target proteins to function while minimizing unwanted interactions. Such considerations become important in AND-Gate bifunctional protein systems that bind to different receptors abundant on different cell types. EPO-Rs are found on numerous cell types besides RBC precursors, including platelet precursors, vascular endothelial cells, liver cells, and white blood cells such as macrophages.

Herein this problem is addressed for “Targeted EPO” molecules, fusion proteins that bind to GPA and weakly to EPO-R and are thus AND-gated to bind to only cells with both of these transmembrane proteins—namely late RBC precursors. The design goal is to activate EPO-R only on late RBC precursors as a result of initial binding to GPA, followed by binding in cis to EPO-R due to the high local concentration of EPO (FIG. 25B). It was found that:
1. Some Targeted EPO proteins mediate adhesion between mature RBCs that express only GPA and other cells that express only EPO-R.
2. The position of the epitope on GPA and the length of the linker between the GPA binding element and the EPO element determines whether a Targeted EPO mediates cell adhesion.
3. The cell adhesion results can be interpreted with a structural model in which GPA extends from the RBC surface and limits how close another cell can approach. To mediate cell adhesion, the EPO element must extend beyond the membrane-distal end of GPA.
4. More broadly, cell-cell interaction may be regulated by abundant cell surface proteins that may sterically prevent close approach of cells. This needs to be considered when constructing engineered cell interaction systems, such as CAR-T cells and bispecific antibodies.

A major challenge for synthetic biology is that artificial biological systems have the potential for undesired interactions that can lead to system failure. When we study natural systems, we are generally not aware of all the behaviors they could have if they were poorly designed. These experiments illustrate geometric principles that natural systems may use in achieving specificity.

The data of FIG. 31A compare bleeding time measurements in mice treated with vehicle to those treated with Epoetin alpha (commercial recombinant human EPO), darbepoetin (a long half-life derivative of EPO), and various forms of targeted EPO synthesized as part of this work. The mouse bleeding time assay is known to be quite noisy and has not previously been used in FVB mice. Moreover, the thrombosis-enhancing effects of positive control proteins, EPO and darbepoetin, did not rise to the level of statistical significance in any single experiment; significance values are determined by combining vehicle-normalized data across experiments. Nonetheless, the initial candidate Targeted EPO protein, 10F7-EPO, appeared to be more thrombotic than these positive controls in two experiments (Experiments 4 and 5), while our expectation was that this protein should have no effect. At a rigorous statistical level these results may not have proven that 10F7-EPO was more thrombotic than vehicle, but the results were concerning enough for us to search for potential mechanisms by which 10F7 might have a thrombotic effect.

It is also noted that the bleeding time measurements were performed one day after injection of the EPO proteins. At this time, new RBCs have not appeared in the blood, so we ascribe any thrombotic effects to direct effects of the molecules on the clotting system and they are likely not due to indirect effects of increased blood viscosity caused by an increase in RBCs. The final motivation for re-investigating 10F7-EPO was a combination of these data, indications of enhancement of RBC stiffness due to 10F7 in the literature that suggested RBC inflammation,^{6,21} and the possibility of cross-linking of cells.

Methods

Methods for cell culture, DNA construction, protein expression and purification, in vitro measurement of erythropoietin activity by cell proliferation assays, measurement of mouse reticulocytes and reticulated platelets, FRAP were performed as described previously.^{4,16}

EPO accessibility and RBC crosslinking measurement by flow cytometry. RBCs (Zen-Bio, Research Triangle Park, N.C.; 0.2% v/v in PBS-glucose) were incubated with fusion proteins for 1 hr at 4° C., washed once with PBS-glucose (PBS, 5 mM glucose, pH 7.4), incubated with anti-His₆-PE antibody (Abcam) for 30 min at 4° C., and washed once with PBS-glucose. Fusion protein concentration used was calculated based on the K_{D} of the monovalent 10F7 and IH4 antibody elements (95 nM and 33 nM, respectively) to achieve 80% saturation of GPA. The cells were analyzed by flow cytometry.

RBC rosette assay. A2780 and MCF-7 cells were plated on a 6-well plate containing 12×12 mm coverslips and incubated overnight at 37° C. On the next day, a mixture of RBCs (0.2% v/v) and fusion proteins was prepared in RPMI-1640 without bicarbonate and incubated for 30 min at 23° C. Coverslips with cells were washed twice with PBS and transferred to a new 6-well plate. The RBC-fusion protein mixture was added on top of each coverslip and incubated for 1 hr at 37° C. Coverslips were gently washed twice with PBS. Rosettes were imaged on a Nikon Eclipse TE300™ inverted phase-contrast microscope. Rosettes were defined as adherent cells bound by 3 or more RBCs among at least 160 cells from at least 8 fields.

Mouse tail vein bleeding time measurement. All mouse experiments were performed under the protocol IS00000723, approved by the Harvard Medical School IACUC. Experiments were performed in a blinded manner. Ten mice per dose group received a single ip injection with saline or test protein. On the next day, mice were anaesthetized. The tail was transected 3 mm from the tip, and the body of the mouse was placed on a heated pad over a 37° C. water bath containing tubes with 50 mL of saline, such that 0.5-2 mm of the transected tail was placed in the saline within 2 seconds of transection. The bleeding time was recorded when bleeding stopped for one minute. Bleeding time longer than 10 minutes were recorded as 10 minutes.

Cell culture. FreeStyle™ 293-F, FreeStyle™ CHO-S, and CHO DG44 cells were obtained from Invitrogen (Carlsbad, Calif.), and were cultured in FreeStyle™ 293 Expression Medium, complete FreeStyle™ CHO Expression Medium, and complete CD DG44 (Invitrogen), respectively. Human erythroleukemia TF-1, human ovarian cancer A2780, and human breast cancer MCF-7 were obtained by ATCC (Manassas, Va.). TF-1 was cultured in RPMI-1640 with 10% FBS, 100 U/mL penicillin, 100 U/mL streptomycin, and 2 ng/mL recombinant human granulocyte macrophage colony-stimulating factor (GM-CSF; PeproTech) unless specified otherwise. A2780 was cultured in RPMI-1640 with 10% FBS. MCF-7 was cultured in DMEM with 10% FBS and 0.01 mg/mL recombinant human insulin (PeproTech). 293-F, CHO-S, and CHO DG44 were cultured at 37° C. in 8% CO₂ with shaking at 2.35×g. TF-1, A2780, and MCF-7 were cultured at 37° C. in 5% CO₂.

DNA constructs/The DNA sequences for 10F7 and EPO (wildtype and mutants) were from GenBank (accession no. KX026660-3). The DNA sequences for IH4, R18, and 1C3 were derived by reverse translating and codon optimizing (Integrated DNA Technologies) the protein sequences adapted from the U.S. Pat. Nos. 9,879,090, 8,900,592, and patent application WO1993024630, respectively.¹⁻³ The IH4 sequence was modified to include a point mutation (Phe80Tyr) and an additional amino acid (Thr118) in the framework regions 3 and 4, respectively, as the reported sequence of IH4 may have had fortuitous changes from the germline sequence or typographical errors. The IH4 antibody with the original sequence in the patent was denoted as IH4*. The DNA sequences for glycine-serine linkers of various lengths were codon-optimized for expression in mammalian cells.

Protein expression and purification. Transient expression was performed in 293-F and CHO-S cells using pSecTag2A or pOptiVEC plasmids, and stable expression was performed using CHO DG44 cells using pOptiVEC plasmids according to the supplier's protocol. Protein expression was assayed by Western blotting cell supernatant using anti-His₆-HRP antibody (Abcam). Stably transfected cells were selected by hypoxanthine/thymidine (HT)-deficient CD OptiCHO medium (Invitrogen), and were subjected to one round of methotrexate (MTX; Sigma-Aldrich) genomic amplification as described before.⁴ Proteins from transient transfection or stable pools were purified as follows. Supernatant was concentrated to 5-8 mL using a 10 kDa cut-off Macrosep Advance centrifugal device (Pall). Concentrated protein was bound to 0.5-1 mL of His60 nickel or HisTalon cobalt resin (Takara Bio) for 0.5 h at 4° C. while rotating in a 10-mL Pierce disposable column (Thermo Scientific), and was washed and eluted using His60 or HisTalon Buffer Set (Takara Bio) according to the supplier's protocol. Cell supernatant and each purification fraction were analyzed by SDS-PAGE followed by Coomassie Blue staining. Eluted proteins were combined, desalted into endotoxin-free PBS (Teknova: 137 mM NaCl, 1.4 mM KH₂PO₄, 4.3 mM Na₂HPO₄, and 2.7 mM KCl, pH 7.4) using Econo-Pac 10DG columns (Bio-Rad), and concentrated to <1 mL using Macrosep Advance centrifugal device. For in vivo experiments, contaminating proteins were further removed by size exclusion chromatography (SEC) on Superdex 200 10/300 GL columns (GE Healthcare) using AKTA FPLC system (GE Healthcare) and endotoxin-free PBS as the running buffer. Desired protein fractions were combined and concentrated to <1 mL using Macrosep Advance centrifugal device. Proteins were stored at 4° C. throughout the described process, ultimately stored as aliquots at −80° C., and thawed once before use. Only endotoxin-free reagents were used.

Cell proliferation assays. TF-1 cells were seeded in a 96-well plate at 9.0×10³ cells per well in 90 μL of RPMI-1640 with serum and antibiotics (no GM-CSF). The purified proteins were serially diluted by 10-fold (10⁻⁷ to 10⁻¹⁴ M) and added to the cells. After 72 hours, cell proliferation was measured by CellTiter 96® AQᵤₑₒᵤₛ One Solution Cell Proliferation Assay (Promega). Absorbance at 490 nm was read on a BioTek Synergy Neo HTS™ microplate reader. Reported data represent mean±SEM of three replicates.

Measurement of mouse reticulocyte and reticulated platelet counts by flow cytometry. The homozygous huGPA transgene is embryonic-lethal but heterozygotes are phenotypically normal, so a breeding colony was maintained with screening for huGPA at each generation. Transgene expression was measured as described before.⁴

In experiments that measure efficacy of fusion and control proteins, five mice per dose group received a single intraperitoneal (ip) injection with saline, darbepoetin, or Targeted EPO in a 200 μL volume (diluted in saline or PBS) on Day 0. 1-5 μL of whole blood was collected by tail-nick in EDTA-coated tubes on days 0, 4, and 7 post-injection. Blood was analyzed immediately after collection by flow cytometry as described before.⁴ Thiazole orange (Sigma-Aldrich) was used to stain residual RNA in reticulocytes and reticulated platelets, and anti-CD41-PE antibody (BD Pharmingen) was used to stain total platelets. A stock solution (1 mg/mL) of thiazole orange was prepared in 100% methanol and was diluted 1:5,000 in PBS to make a 2× working solution. Anti-CD41-PE antibody was diluted 1:500 in either the 2× working solution of thiazole orange for stained samples or PBS for gating thiazole orange-negative population. 2 μL of whole blood was diluted 1:1,000 in 2 mL of PBS. Equal volumes (100 μL) of 2× working solution of anti-CD41-PE antibody with or without thiazole orange and diluted whole blood were mixed in a 96-well U-bottom plate and incubated for 30 min in the dark at RT. The fluorescence was measured on a LSRFortessa™ SORP flow cytometer equipped with an optional HTS sampler (BD Biosciences) using the following filter configuration: PE excitation, 561/50 mW; emission filter, BP 582/15; YFP excitation, 488/100 mW; emission filter, BP 540/25.

Preparation of human red blood cells. For FRAP, blood was obtained from healthy adult volunteers in accordance with the guidelines of, and approved by the Institutional Review Board of Beth Israel Deaconess Medical Center. Human blood was collected into HBSS++ containing 0.05% IgG-free BSA by venipuncture. RBCs were centrifuged at 5,000×g at 23° C. for 1 min, and were washed once with the same buffer. For flow cytometry and rosetting assays, human whole blood collected in an ACD tube by venipuncture was purchased from Zen-Bio (Research Triangle Park, N.C.). RBCs were isolated by centrifugation at 5,000×g at 23° C. for 1 min, and washed twice with PBS-glucose (PBS, 5 mM glucose, pH 7.4).

Fluorescence recovery after photobleaching. We used fluorescence recovery after photobleaching (FRAP) to assess whether various antibody elements induced an ‘inflamed’ state in red blood cells.

The binding of several anti-GPA antibodies is known to cause pro-inflammatory phenotypes in RBCs, such as production of reactive oxygen species (ROS), phosphorylation of band 3, rearrangement of RBC skeletal proteins, increase in RBC membrane stiffness, and decrease in RBC membrane fluidity.^{6,7} RBC membrane deformability and lipid mobility is one of the most frequently used readouts for such changes in RBCs upon treatment with stimuli, such as anti-GPA antibodies and complement. Khoory et al., 2016 illustrated that binding of particular anti-GPA antibodies, R10 or E4, induced an increase in intracellular ROS, ATP release, RBC rigidity, a decrease in membrane deformability, and a decrease in the mobile lipid fraction as measured by FRAP.⁸ Piagnerelli et al., 2007 showed that RBCs of septic patients had altered lipid bilayers and decreased membrane deformability.⁹

FRAP is a technique to measure membrane fluidity in mammalian cells such as RBCs. In this technique, cells are incubated with a lipid-soluble fluorescent dye to evenly label the membranes, and then a region of the membrane is bleached with a laser. The non-bleached dye from adjacent membrane will diffuse back into the bleached area at a certain rate that is a measure of the membrane fluidity. Decreased membrane fluidity is correlated with RBC inflammation and may be a direct consequence of cell stiffness that results from cytoskeletal changes upon inflammatory signaling.^{8,10} For example, movement of proteins such as GPA and Band 3, whose transmembrane segments occupy a significant fraction of the RBC membrane, become immobilized and slow the movement of the surrounding lipids.

RBCs (10% v/v in HBSS++ containing 0.1% IgG-free BSA) were incubated with lipid dye DiO (15 μg/mL) for 30 min at 23° C. in dark and washed once with HBSS++ containing 0.1% IgG-free BSA. Then RBCs were treated with GPA antibodies at 37° C. for 15 min. The concentration of antibody was determined by its K_{D} to achieve 80% saturation of GPA. RBCs were imaged using a 60× objective on an Olympus BX62 fluorescence microscope, bleached with Vector Controller Laser, and analyzed as described before.^{8,11} Reported data represent mean±95% confidence interval of 8-9 cells (experiment 1) or 16-20 cells (experiment 2). Data from each experiment were normalized to the buffer control in the same experiment to account for day-to-day variability, and the normalized values were combined across experiments. Mann-Whitney test was performed to determine p-values for individual and combined data sets.

Flow cytometry for EPO accessibility, EPO binding, and RBC crosslinking measurement. The fluorescence was measured on a LSRFortessa™ SORP flow cytometer equipped with an HTS sampler (BD Biosciences) using the following filter configuration: PE excitation, 561/50 mW; emission filter, BP 582/15. Data were analyzed using FlowJo™ Data Analysis software (TreeStar, Ashland, Oreg.). Reported data represent mean±SEM of three replicates.

RBC rosette assay/The rosette assay is a particularly sensitive way to demonstrate the presence of surface receptors expressed at low levels.¹² We adapted this assay to study cell-cell adhesion mediated by fusion proteins that bind to RBCs. A2780 and MCF-7 cells were plated at 2.0×10⁵ cells/mL in a 2 mL volume on a 12×12 mm coverslip (Electron microscopy sciences) in a well of a 6-well plate (Corning) and incubated overnight at 37° C. in 5% CO₂. The following day, a mixture of RBCs (0.2% v/v) and fusion proteins was prepared in binding medium (RPMI-1640 without bicarbonate) and incubated for 30 min at 23° C. Coverslips coated with cells were washed twice with PBS and transferred to a new 6-well plate. The RBC-fusion protein mixture (200 μL) was added on top of each coverslip and incubated for 1 hr at 37° C. Coverslips were gently washed twice with PBS. Rosettes were imaged on a Nikon Eclipse TE300 inverted phase-contrast microscope (Nikon, Melville, N.Y.), using a Retiga EXi™ CCD camera (QImaging, Surrey, Canada) controlled by iVision 4.7 software (BioVision). Rosettes were defined as adherent cells bound by 3 or more RBCs and were shown as a percentage of at least 160 cells from at least 8 fields. Reported data represent mean±SEM of at least two replicates

Measurement of EPO binding in a mixed cell population by flow cytometry/A2780 cells were harvested using 0.25% trypsin and resuspended in the FACS staining buffer (R&D Systems, Minneapolis, Minn.). A2780 (1.0×10⁶ cells), RBCs (2.0×10⁶ cells) and fusion proteins (250 nM) were mixed together, let tumble gently for 30 min at RT, and then stained with anti-His₆-PE antibody (Abcam) for 30 min at RT. The cells were analyzed by flow cytometry.

Mouse tail vein bleeding time measurement. Dose levels were chosen based on doses that were the minimum required to achieve an increase of reticulocytes of 10% of total RBCs relative to baseline 4 days after injection. For example, in a typical FVB mouse the fraction of reticulocytes is about 5%. In a mouse with a 5% reticulocyte baseline at day 0, an increase to 15% would be recorded as 10% relative to baseline. Four days is the point at which an increase in reticulocytes represents the maximal response. The potency of a given EPO-based molecule is a complex function of affinity for receptor, rate of receptor internalization and degradation during signaling, pharmacokinetics and distribution, and possible inflammatory effects that might antagonize erythropoiesis. Thus, different molecules were compared at functionally equivalent therapeutic doses. We found that doses of 1.8 μg of darbepoetin, 9 μg of 10F7-35-EPO(R150A), and 2 μg of IH4-5-EPO(R150A) had equivalent erythropoietic activity.

The tail transection assay of bleeding time was generally performed on mice as follows. On day 0, mice were injected with a test protein (or vehicle). When testing an unknown protein, it is important to also include mice that are injected with saline or PBS vehicle as a negative control, and EPO or darbepoetin as a positive control. Typically 10 mice per dose group were used. The experiments were performed in a blind manner: one experimenter performed the injections of proteins into the mice, maintained the key, performed the injection of anaesthetic, and then handed the mice in a random order to a second, blinded experimenter. The second experimenter performed the tail transections and measured the bleeding time. For most experiments, on day 1 the tail transection and bleeding time was measured. In some experiments, the experiment was performed on day 4 so that changes in reticulocytes and reticulated platelets could also be measured. However, the advantage of performing the measurement 1 day after treatment is that after only 24 hours, the level of circulating red blood cells will not have changed, so effects on blood clotting are due to direct effects on some element of the clotting system, and not due to changes in blood viscosity.

Tail transection was performed as follows. The mice were first anaesthetized using anaesthetics ketamine and xylazine. These anaesthetics are chosen because they are thought to not affect blood clotting. Acepromazine is not used because it has the effect of reducing clotting. Mice were weighed, and mice were then injected with 120-160 mg ketamine/kg and 10-16 mg xylazine/kg of body weight. For older and heavier mice, sometimes an additional injection of about 25% of the first injection was required. After a mouse became unresponsive to a stimulus such as significant pressure to a hind foot, the mouse was placed on a heated pad on a platform over a water bath. The water bath was maintained at 37° C. 50-mL blue-cap tubes (Sarstedt) were filled with 50 mL of a solution of 0.85 to 0.9% NaCl that has been equilibrated to 37° C. in a separate water bath. The animal was placed on a Chux pad for the transection.

A position on the tail that is 3 mm from the tip, not counting hair, was marked with a felt-tip pen using calipers. (The tail was also inspected for signs of bruising that may be due to fighting, and data from such a mouse was discarded if the transected tail did not bleed at all. The decision to discard the data was always made in a blinded manner at the time of measurement). The tail was transected with a flat razor blade using a section of the blade that has not been used previously. Within 1-2 seconds, the transected tail was placed in a tube with pre-warmed saline, and then the body of the mouse was placed on the heated pad above the water bath. At the moment that the tail was transected by the blinded experimenter, the non-blinded experimenter started a timer. The body of the mouse was then positioned on the heated pad so that only the tip of the tail—about 0.5 to 2 mm—is in the saline and the rest is in the air. When observing the bleeding tail, the tube was rotated so that the white stripe was behind the tail, providing contrast, and the room was well-lit. The rack holding the 50-mL tube was white or yellow to provide contrast. The bleeding time was recorded by noting when bleeding stops, and then observing the submerged tail for up to one minute. If bleeding re-started within this minute, the first recorded time was not counted. Bleeding may stop and re-start several times. If the tail is still bleeding when 10 minutes have elapsed, the time was recorded as 10 minutes.

The median and mean bleeding times were calculated for each treatment group. Calculating the median has the advantage that extreme events, such as 10-minute time points, do not disproportionately contribute to the calculation. Data from each experiment were normalized to the vehicle control in the same experiment to account for day-to-day variability. The normalized values were combined across experiments and Mann-Whitney test was performed to determine p-values of the combined data sets. Specifically, the mean of vehicle-treated controls was calculated for each experiment, and then individual data points for vehicle- and protein-treated animals were normalized to these mean values. Thus, the variability in the vehicle control samples was preserved in calculating possible statistical significance. In the summary of these data shown in Figure S1c, the normalized data from multiple bleeding time experiments were combined to estimate the overall magnitude and statistical significance of the treatment effects.

Methods of estimating molecular distances. Molecular distances as schematically depicted in FIG. 28F and as discussed in the text were estimated as follows. The extracellular domain of GPA is generally described as an intrinsically unstructured protein, based on the fact that the first 50 amino acids of the protein include 17 O-glycosylation sites that are variably modified and one N-linked glycosylation site; these are so densely packed that formation of a hydrophobic core is likely precluded in this region, particularly for amino acids 1-33.¹³⁻¹⁵

The O-linked oligosaccharides are expected to extend at least about 20 Angstroms from the peptide backbone. In the crystal and NMR structures of glycosylated proteins, the average distance between the peptide backbone and the farthest end of a single disaccharide unit is about 8-10 Angstroms. Therefore, an N-/O-linked glycan is estimated to extend about 20-25 Angstroms from the peptide backbone.^{16,17}

Taken together, at least the first 33 amino acids are expected to form an extended coil, similar in conformation to a beta-strand and with about 3-3.5 Angstroms per amino acid. For example, in the GFP structure PDB ID: 1C4F, the length of the Asn159-Gln172 beta strand is about 42 Angstroms. Thus, the distance from the 10F7 epitope (roughly amino acid 33) to the N-terminus is likely at least 100 Angstroms.

The length of a fully extended glycine-serine linker of 35 amino acids would also be about 100 Angstroms. The flexible glycine-serine linker connecting the antibody and EPO elements is thought to form a random coil with a length distribution approximating that of a volume-excluded Brownian walk.¹⁸ However, when the antibody element is bound, the EPO element likely explores all of the available space on a short timescale. Thus, if the EPO element binds to an EPO-R while the linker is in a completely extended conformation, that conformation will be stabilized. More precisely, when the antibody element is bound to GPA, the concentration of a single molecule of attached EPO in a sphere with a 100 Angstrom radius is about 400 μM. Since the dissociation constant (K_{D}) of EPO(R150A) for EPO-R is about 81 nM,⁴ it is expected that if an EPO-R is found within the radius afforded by an extended linker, binding will occur.

The size, shape, and binding angle of the anti-GPA antibody V regions 10F7 and IH4 can be estimated from the structures of other scFv's and nanobodies, and from steric considerations of how bound antibodies on the surface of B cells could interact with epitopes on GPA during antibody selection. It is important to recall that when a B cell expressing a particular antibody is being positively selected, that B cell engages the antigen through membrane-bound antibodies that are brought in proximity.¹⁹ The 10F7 and IH4 antibodies were both developed by immunization with whole human RBCs. The length of an antibody Fc region is about 80-90 Angstroms, so that membrane-bound antibody could not be in the T conformation during antigen recognition. Instead, the Fab portion must be angled upward, away from the RBC membrane. As a result, the net displacement from the CDRs of the V region(s) to the C-terminus of the V region(s) would need to be at least 10 Angstroms and up to 45 angstroms (the length of a V region.)

In the solved structures of EPO complexed with its receptor dimer (PDB ID: 1EER and 1CN4), EPO is roughly a prolate spheroid with its long axis parallel to the membrane of the EPO-R-bearing cell. In a configuration in which the EPO-Rs are maximally extended from the membrane, the N-terminus of EPO would be about 50 Angstroms from the membrane. The N-terminus of EPO is at least about 25 Angstroms from the most membrane-distal amino acids on the bound EPO-R, so if an EPO fusion protein is bound to GPA to a RBC, a linker would need to span this distance plus the distance from where it is anchored on the RBC. This reasoning assumes that EPO-R on one cell and GPA on another cell cannot interdigitate; this seems a reasonable assumption because of the glycosylation of GPA.

The IH4 nanobody element recognizes the sequence ₅₂YPPE₅₅ in GPA.²⁰ This is about 20 amino acids C-terminal to the likely epitope of 10F7.^{6,21,22}

Taken together, these estimates indicate that for 10F7-35-EPO(R150A) when 10F7 is bound to GPA on a RBC, the surface of EPO that binds to its receptor can protrude at least 10 Angstroms beyond the N-terminus of GPA even if this protein is in a maximally extended conformation. For IH4-5-EPO(R150A), the EPO element is about 150 Angstroms closer to the RBC membrane, corresponding to a 20-amino acid more membrane proximal epitope, and a linker that is 30 amino acids shorter.

### REFERENCES

(1) Goebeler, M. E., and Bargou, R. (2016) Blinatumomab: a CD19/CD3 bispecific T cell engager (BiTE) with unique anti-tumor efficacy. Leuk Lymphoma 57, 1021-1032.
(2) Sondel, P. M., and Gillies, S. D. (2012) Current and Potential Uses of Immunocytokines as Cancer Immunotherapy. Antibodies (Basel) 1, 149-171.
(3) Chang, Z. L., and Chen, Y. Y. (2017) CARs: Synthetic Immunoreceptors for Cancer Therapy and Beyond. Trends Mol Med 23, 430-450.
(4) Burrill, D. R., Vernet, A., Collins, J. J., Silver, P. A., and Way, J. C. (2016) Targeted erythropoietin selectively stimulates red blood cell expansion in vivo. Proc Natl Acad Sci USA 113, 5245-5250.
(5) Taylor, N. D., Way, J. C., Silver, P. A., and Cironi, P. (2010) Anti-glycophorin single-chain Fv fusion to low-affinity mutant erythropoietin improves red blood cell-lineage specificity. Protein Eng Des Sel 23, 251-260.
(6) Merry, A. H., Thomson, E. E., Anstee, D. J., and Stratton, F. (1984) The quantification of erythrocyte antigen sites with monoclonal antibodies. Immunology 51, 793-800.
(7) Wickrema, A., and Crispino, J. D. (2007) Erythroid and megakaryocytic transformation. Oncogene 26, 6803-6815.
(8) Anagnostou, A., Lee, E. S., Kessimian, N., Levinson, R., and Steiner, M. (1990) Erythropoietin has a mitogenic and positive chemotactic effect on endothelial cells. Proc Natl Acad Sci USA 87, 5978-5982.
(9) Lifshitz, L., Tabak, G., Gassmann, M., Mittelman, M., and Neumann, D. (2010) Macrophages as novel target cells for erythropoietin. Haematologica 95, 1823-1831.
(10) Chasis, J. A., and Mohandas, N. (1992) Red blood cell glycophorins. Blood 80, 1869-1879.
(11) Knowles, D. W., Chasis, J. A., Evans, E. A., and Mohandas, N. (1994) Cooperative action between band 3 & glycophorin A in human erythrocytes: immobilization of band 3 induced by antibodies to glycophorin A. Biophysical Journal 66, 1726-1732.
(12) Paulitschke, M., Nash, G. B., Anstee, D. J., Tanner, M. J. A., and Gratzer, W. B. (1995) Perturbation of red blood cell membrane rigidity by extracellular ligands. Blood 86, 342-348.
(13) Giger, K., Habib, I., Ritchie, K., and Low, P. S. (2016) Diffusion of glycophorin A in human erythrocytes. Biochim Biophys Acta 1858, 2839-2845.
(14) Habib, I., Smolarek, D., Hattab, C., Grodecka, M., Hassanzadeh-Ghassabeh, G., Muyldermans, S., Sagan, S., Gutierrez, C., Laperche, S., Le-Van-Kim, C., Aronovicz, Y. C., Wasniowska, K., Gangnard, S., and Bertrand, O. (2013) V(H)H (nanobody) directed against human glycophorin A: a tool for autologous red cell agglutination assays. Anal Biochem 438, 82-89.
(15) Gardner, B., Parsons, S. F., Merry, A. H., and Anstee, D. J. (1989) Epitopes on sialoglycoprotein a: evidence for heterogeneity in the molecule. Immunology 68, 283-289.
(16) Khoory, J., Estanislau, J., Elkhal, A., Lazaar, A., Melhom, M. I., Brodsky, A., Illigens, B., Hamachi, I., Kurishita, Y., Ivanov, A. R., Shevkoplyas, S., Shapiro, N. I., and Ghiran, I. C. (2016) Ligation of Glycophorin A Generates Reactive Oxygen Species Leading to Decreased Red Blood Cell Function. PLoS One 11, e0141206.
(17) Paragh, G., Kumar, S. M., Rakosy, Z., Choi, S. C., Xu, X., and Acs, G. (2009) RNA interference-mediated inhibition of erythropoietin receptor expression suppresses tumor growth and invasiveness in A2780 human ovarian carcinoma cells. Am J Pathol 174, 1504-1514.
(18) Acs, G., Acs, P., Beckwith, S. M., Pitts, R. L., Clements, E., Wong, K., and Verma, A. (2001) Erythropoietin and Erythropoietin Receptor Expression in Human Cancer. Cancer Res 61, 3561-3565.
(19) Ballas, S. K. (1987) Erythrocyte concentration and volume are inversely related. Clinica Chimica Acta 164, 243-244.
(20) Kong, L., Wilson, I. A., and Kwong, P. D. (2015) Crystal structure of a fully glycosylated HIV-1 gp120 core reveals a stabilizing role for the glycan at Asn262. Proteins 83, 590-596.
(21) Schuster, O., Klich, G., Sinnwell, V., Kranz, H., Paulsen, H., and Meyer, B. (1999) ‘Wave-type’ structure of a synthetic hexaglycosylated decapeptide: A part of the extracellular domain of human glycophorin A. Journal of Biomolecular NMR 14, 33-45.
(22) Kirkeby, A., Torup, L., Bochsen, L., Kjalke, M., Abel, K., Theilgaard-Monch, K., Johansson, P. I., Bjorn, S. E., Gerwien, J., and Leist, M. (2008) High-dose erythropoietin alters platelet reactivity and bleeding time in rodents in contrast to the neuroprotective variant carbamyl-erythropoietin (CEPO). Thromb Haemost 99, 720-728.

1. A polypeptide comprising a) an anti-GYPA antibody reagent that binds the epitope of SEQ ID NO: 25; b) an erythropoietin; and c) a linker sequence separating the anti-GYPA antibody reagent and the erythropoietin.

2. (canceled)

3. The polypeptide of claim 1, wherein the anti-GYPA antibody reagent comprises the three CDRs of IH4.

4. The polypeptide of claim 1, wherein the anti-GYPA antibody reagent comprises a VHH having the sequence of SEQ ID NO: 10 or 11.

5. (canceled)

6. The polypeptide of claim 1, wherein the anti-GYPA antibody reagent comprises the CDRs of an antibody reagent selected from R18, IH4, IH4v1, and Table 3.

7. The polypeptide of claim 1, wherein the anti-GYPA antibody reagent comprises the VH and VL sequences of an antibody reagent selected from R18, IH4, IH4v1, and Table 3.

8. The polypeptide of claim 1, wherein the anti-GYPA antibody reagent comprises an antibody reagent selected from R18, IH4, IH4v1, and Table 3.

9. The polypeptide of claim 6, wherein the antibody reagent is selected from R18, IH4, IH4v1, 2B-21, 2B-25, 2B-9, 2B-20, 2B-19, NaM70-3C10, 2B-4, B14, B89, R7, and BRIC 93.

10. The polypeptide of claim 6, wherein the antibody reagent is selected from IH4, IH4v1, 2B-9, 2B-20, 2B-19, NaM70-3C10, 2B-4, B14, B89, R7, and BRIC 93.

11. The polypeptide of claim 1, wherein the erythropoietin comprises at least one mutation at an amino acid residue of SEQ ID NO: 16 selected from R150, A30, H32, P87, W88, P90, R53, and E55.

12. The polypeptide of claim 11, wherein the at least one mutation is R150A, A30N, H32T, P87V, W88N, P90T, R53N, or E55T.

13. The polypeptide of claim 1, wherein the linker sequence is no more than 17 amino acids in length.

14. The polypeptide of claim 1, wherein the linker sequence is at least 5 amino acids in length.

15. (canceled)

16. (canceled)

17. (canceled)

18. A polypeptide comprising an anti-GYPA antibody reagent; a linker sequence of no more than 17 amino acids; and an activity element.

19.-34. (canceled)

35. A method of increasing erythropoiesis comprising contacting a red blood cell with a polypeptide of claim 1.

36. A method of treating anemia in a subject in need thereof, the method comprising administering a polypeptide of claim 1 to the subject.

37. (canceled)

38. The method of claim 36, wherein the subject has or is diagnosed as having chronic renal failure or altitude sickness or has received chemotherapy.

39. A nucleic acid or vector encoding the polypeptide of claim 1.

40. (canceled)

41. A cell comprising the nucleic acid or vector of claim 39.
